(19) United States
(12) Patent Application Publication
(10) Pub. No.: US 2009/0047244 A1
(43) Pub. Date: Feb. 19, 2009

(54) MACROCYCLIC SERINE PROTEASE INHIBITORS I

(75) Inventors: Christophe Claude Parsy, Jacou (FR); Dominique Surleraux, Wauthier-Braine (BE); Francois-Rene Alexandre, Montpellier (FR); Michel Derock, Montpellier (FR); Frederic Leroy, Nontarnaud (FR)

Correspondence Address: JONES DAY, 222 EAST 41ST ST, NEW YORK, NY, 10017, US

(73) Assignee: Idenix Pharmaceuticals, Inc., Cambridge, MA (US)

(21) Appl. No.: 12/180,446

(22) Filed: Jul. 25, 2008

Related U.S. Application Data
(60) Provisional application No. 60/962,435, filed on Jul. 26, 2007.

(51) Int. Cl. A61K 38/20; C07D 417/14; A61K 31/4709; A61K 38/21; C12N 9/99; C12N 7/06; A61K 31/7088; A61K 31/395

(52) U.S. Cl. 424/85.2; 540/460; 514/312; 514/601; 514/44; 424/85.4; 424/85.7; 424/85.6; 435/238; 435/183

(57) ABSTRACT

Provided herein are macrocyclic serine protease inhibitor compounds, for example, of Formula I, pharmaceutical compositions comprising such compounds, and processes of preparation thereof. Also provided are methods of their use for the treatment of an HCV infection in a host in need thereof.

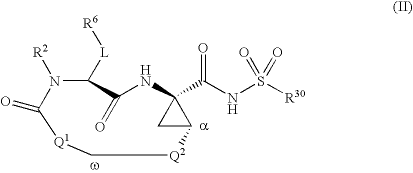

### CROSS REFERENCE TO RELATED APPLICATION

This application claims the benefit of the priority of U.S. Provisional Application No. 60/962,435, filed Jul. 26, 2007, the disclosure of which is incorporated herein by reference in its entirety.

### FIELD

Provided herein are macrocyclic serine protease inhibitor compounds, pharmaceutical compositions comprising such compounds, and processes of preparation thereof. Also provided are methods of their use for the treatment of an HCV infection in a host in need thereof.

### BACKGROUND

Hepatitis C virus (HCV) is known to cause at least 80% of posttransfusion hepatitis and a substantial proportion of sporadic acute hepatitis (Houghton et al., Science 1989, 244, 362-364; Thomas, Curr. Top. Microbiol. Immunol. 2000, 25-41). Preliminary evidence also implicates HCV in many cases of “idiopathic” chronic hepatitis, “cryptogenic” cirrhosis, and probably hepatocellular carcinoma unrelated to other hepatitis viruses, such as hepatitis B virus (Di Besceglie et al., Scientific American, 1999, October, 80-85; Boyer et al., J. Hepatol. 2000, 32, 98-112).

HCV is an enveloped virus containing a positive-sense single-stranded RNA genome of approximately 9.4 kb (Kato et al., Proc. Natl. Acad. Sci. USA 1990, 87, 9524-9528; Kato, Acta Medica Okayama, 2001, 55, 133-159). The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

Presently, the most effective HCV therapy employs a combination of alpha-interferon and ribavirin, leading to sustained efficacy in about 40% of patients (Poynard et al., Lancet 1998, 352, 1426-1432). Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load (Manns et al, Lancet 2001, 358, 958-965; Fried et al., N. Engl. J. Med. 2002, 347, 975-982; Hadziyannis et al., Ann. Intern. Med. 2004, 140, 346-355). Thus, there is a clear and unmet need to develop effective therapeutics for treatment of HCV infection.

### SUMMARY OF THE DISCLOSURE

Provided herein are macrocyclic serine protease inhibitor compounds, pharmaceutical compositions comprising such compounds, and processes of preparation thereof. Also provided are methods of the use of the compounds for the treatment of an HCV infection in a host in need thereof.

In one embodiment, provided herein is a compound of Formula I:

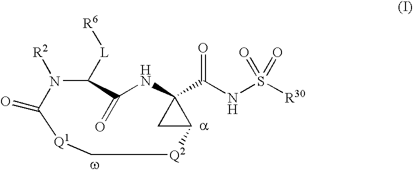

or a single enantiomer, a mixture of enantiomers, an individual diastereomer, or a mixture of diastereomers thereof; or a pharmaceutically acceptable salt, solvate, or prodrug thereof;
wherein:

R² is hydrogen, C₁₋₆ alkyl, C₃₋₇ cycloalkyl, C₆₋₁₄ aryl, heterocyclyl, or heteroaryl;

R⁶ is hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl;

R³⁰ is hydrogen, C₁₋₆ alkyl, C₂₋₆ alkenyl, C₂₋₆ alkynyl, C₃₋₇ cycloalkyl, C₆₋₁₄ aryl, heteroaryl, heterocyclyl, or C₁₋₆ alkyl-C₃₋₇ cycloalkylene;

L is a bond, C₁₋₆ alkylene, C₂₋₆ alkenylene, C₂₋₆ alkynylene, C₃₋₇ cycloalkyl, or —(CR^{a}R^{b})ₚX—; wherein p is an integer of 0, 1, 2, or 3; R^{a} and R^{b} are each independently hydrogen, halo, cyano, hydroxyl, or alkoxy; and X is —O—, —S—, —C(O)—, —C(O)O—, —OC(O)O—, —C(O)NR¹⁴—, —C(═NR¹⁴)NR¹⁵—, —NR¹⁴C(O)NR¹⁵—, —NR¹⁴C(═NR¹⁵)NR¹⁶—, —NR¹⁴S(O)ₖR¹⁵—, —NR¹⁴S(O)ₖNR¹⁵—, —S(O)ₖ—, —S(O)ₖNR¹⁴—, —P(O)OR¹⁴—, or —OP(O)OR¹⁴—; where R¹⁴, R¹⁵, and R¹⁶ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and each k is independently an integer of 1 or 2;

Q¹ is —O—, —N(R¹⁷)—, —C(R¹⁸R¹⁹)—, or —CR¹⁷(NR¹⁸R¹⁹)—; wherein:
R¹⁷ and R¹⁸ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and
R¹⁹ is hydrogen, C₁₋₆ alkyl, C₂₋₆ alkenyl, C₂₋₆ alkynyl, C₃₋₇ cycloalkyl, C₆₋₁₄ aryl, heterocyclyl, heteroaryl, —C(O)R²⁰, —C(O)OR²⁰, —C(O)NR²¹R²²—C(═NR²⁰)NR²¹R²², or —S(O)ₘR²⁰; where R²⁰, R²¹, and R²² are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R²¹ and R²² are linked together with the N atom to which they are attached to form heterocyclyl or heteroaryl; and m is an integer of 0, 1, or 2; or
R¹⁸ and R¹⁹ are linked together with the C or N atom to which they are attached to form cycloalkyl, heterocyclyl, or heteroaryl; and

Q² is C₃₋₉ alkylene, C₃₋₉ alkenylene, or C₃₋₉ alkynylene, each optionally containing one to three heteroatoms in the chain of the alkylene, independently selected from O, N, and S;

wherein each alkyl, alkylene, alkenyl, alkenylene, alkynyl, alkynylene, cycloalkyl, cycloalkylene, aryl, heteroaryl, and heterocyclyl is independently, optionally substituted with one or more substituents Q, each Q independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} are linked together to form heterocyclyl, along with the N atom to which they are attached.

Also provided herein are pharmaceutical compositions comprising a compound provided herein, e.g., a compound of Formula I, including a single enantiomer, a mixture of enantiomers, an individual diastereomer, or a mixture of diastereomers thereof; or a pharmaceutically acceptable salt, solvate, or prodrug thereof; in combination with one or more pharmaceutically acceptable excipients or carriers.

Further provided herein is a method for treating or preventing an HCV infection, which comprises administering to a subject a therapeutically effective amount of a compound provided herein, e.g., a compound of Formula I, including a single enantiomer, a mixture of enantiomers, an individual diastereomer, or a mixture of diastereomers thereof; or a pharmaceutically acceptable salt, solvate, or prodrug thereof.

Additionally provided herein is a method for treating, preventing, or ameliorating one or more symptoms of a liver disease or disorder associated with an HCV infection, comprising administering to a subject a therapeutically effective amount of a compound provided herein, e.g., a compound of Formula I, including a single enantiomer, a mixture of enantiomers, an individual diastereomer, or a mixture of diastereomers thereof; or a pharmaceutically acceptable salt, solvate, or prodrug thereof.

Provided herein is a method for inhibiting replication of a virus in a host, which comprises contacting the host with a therapeutically effective amount of a compound provided herein, e.g., a compound of Formula I, including a single enantiomer, a mixture of enantiomers, an individual diastereomer, or a mixture of diastereomers thereof; or a pharmaceutically acceptable salt, solvate, or prodrug thereof.

Provided herein is a method for inhibiting the activity of a serine protease, which comprises contacting the serine protease with a compound provided herein, e.g., a compound of Formula I, including a single enantiomer, a mixture of enantiomers, an individual diastereomer, or a mixture of diastereomers thereof; or a pharmaceutically acceptable salt, solvate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The term “host” refers to a unicellular or multicellular organism in which a virus can replicate, including, but not limited to, a cell, cell line, and animal, such as human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disease; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The term “IC₅₀” refers an amount, concentration, or dosage of a compound that is required for 50% inhibition of a maximal response in an assay that measures such response.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder or disease. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “release controlling excipient” refers to an excipient whose primary function is to modify the duration or place of release of an active substance from a dosage form as compared with a conventional immediate release dosage form.

The term “nonrelease controlling excipient” refers to an excipient whose primary function do not include modifying the duration or place of release of an active substance from a dosage form as compared with a conventional immediate release dosage form.

The term “alkyl” refers to a linear or branched saturated monovalent hydrocarbon radical. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl may be substituted.

The term “alkylene” refers to a linear or branched saturated divalent hydrocarbon radical, wherein the alkylene may optionally be substituted. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms). For example, C₂₋₆ alkylene refers to a linear saturated divalent hydrocarbon radical of 2 to 6 carbon atoms or a branched saturated divalent hydrocarbon radical of 3 to 6 carbon atoms.

The term “alkenyl” refers to a linear or branched monovalent hydrocarbon radical, which contains one or more carbon-carbon double bonds. The alkenyl may be optionally substituted, e.g., as described herein. The term “alkenyl” also embraces radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl, allyl, propenyl, butenyl, and

4-methylbutenyl.

The term “alkenylene” refers to a linear or branched divalent hydrocarbon radical, which contains one or more carbon-carbon double bonds. The alkenylene may be optionally substituted, e.g., as described herein. Similarly, the term “alkenylene” also embraces radicals having “cis” and “trans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, propenylene, allylene, propenylene, butenylene, and 4-methylbutenylene.

The term “alkynyl” refers to a linear or branched monovalent hydrocarbon radical, which contains one or more carbon-carbon triple bonds. The alkynyl may be optionally substituted, e.g., as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “alkynylene” refers to a linear or branched divalent hydrocarbon radical, which contains one or more carbon-carbon triple bonds. The alkynylene may be optionally substituted. e.g., as described herein. The term “alkynylene” also encompasses both linear and branched alkynylene, unless otherwise specified. In certain embodiments, the alkynylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched divalent hydrocarbon radical of 3 to 20 (C₃₋₂₀), 3 to 15 (C₃₋₁₅),

3 to 10 (C₃₋₁₀), or 3 to 6 (C₃₋₆) carbon atoms. Examples of alkynylene groups include, but are not limited to, ethynylene (—C≡C—) and propargylene (—CH₂C≡C—). For example, C₂₋₆ alkynyl refers to a linear unsaturated divalent hydrocarbon radical of 2 to 6 carbon atoms or a branched unsaturated divalent hydrocarbon radical of 3 to 6 carbon atoms.

The term “cycloalkyl” refers to a cyclic saturated bridged or non-bridged monovalent hydrocarbon radical, which may be optionally substituted, e.g., as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

The term “cycloalkylene” refers to a cyclic saturated bridged or non-bridged divalent hydrocarbon radical, which may be optionally substituted, e.g., as described herein. In certain embodiments, the cycloalkylene has from 3 to 20 (C₃₋₂₀), from 3 to 15 (C₃₋₁₅), from 3 to 10 (C₃₋₁₀), or from

3 to 7 (C₃₋₇) carbon atoms. Examples of cycloalkylene groups include, but are not limited to, cyclopropylene, cyclobutylene, cyclopentylene, cyclohexylene, cycloheptylene, decalinylene, and adamantylene.

The term “aryl” refers to a monocyclic or multicyclic monovalent aromatic group. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). All such aryl groups may also be optionally substituted, e.g., as described herein.

The term “arylene” refers to a monocyclic or multicyclic divalent aromatic group. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene, naphthylene, fluorenylene, azulenylene, anthrylene, phenanthrylene, pyrenylene, biphenylene, and terphenylene. Arylene also refers to bicyclic or tricyclic carbon rings, where one of the rings is aromatic and the others of which may be saturated, partially unsaturated, or aromatic, for example, dihydronaphthylene, indenylene, indanylene, or tetrahydro-naphthylene (tetralinyl). All such aryl groups may also be optionally substituted, e.g., as described herein.

The term “heteroaryl” refers to a monocyclic or multicyclic aromatic group, wherein at least one ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. All such heteroaryl groups may also be optionally substituted, e.g., as described herein.

The term “heterocyclyl” or “heterocyclic” refers to a monocyclic or multicyclic non-aromatic ring system, wherein one or more of the ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Examples of heterocyclyl groups include, but are not limited to, pyrrolidinyl, piperidinyl, 2-oxopyrrolidinyl, 2-oxopiperidinyl, morpholinyl, piperazinyl, tetrahydropyranyl, and thiomorpholinyl. All such heterocyclic groups may also be optionally substituted, e.g., as described herein.

The term “alkoxy” refers to an —OR radical, wherein R is, for example, alkyl, alkenyl, alkynyl, cycloalkyl, aryl, heteroaryl, or heterocyclyl, each as defined herein. Examples of alkoxy groups include, but are not limited to, methoxy, ethoxy, propoxy, n-propoxy, 2-propoxy, n-butoxy, isobutoxy, tert-butoxy, cyclohexyloxy, phenoxy, benzoxy, and 2-naphthyloxy.

The term “acyl” refers to a —C(O)R radical, wherein R is, for example, alkyl, alkenyl, alkynyl, cycloalkyl, aryl, heteroaryl, or heterocyclyl, each as defined herein. Examples of acyl groups include, but are not limited to, acetyl, propionyl, butanoyl, isobutanoyl, pentanoyl, hexanoyl, heptanoyl, octanoyl, nonanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, arachidonoyl, benzoyl, pyridinylcarbonyl, and furoyl.

The term “halogen”, “halide” or “halo” refers to fluorine, chlorine, bromine, or iodine.

The term “optionally substituted” is intended to mean that a group, such as an alkyl, alkylene, alkenyl, alkenylene, alkynyl, alkynylene, cycloalkyl, cycloalkylene, aryl, arylene, heteroaryl, or heterocyclyl group, may be substituted with one or more substituents independently selected from, e.g., halo, cyano (—CN), nitro (—NO₂), —SR^{a}, —S(O)R^{a}, —S(O)₂R^{a}, —R^{a}, —C(O)R^{a}, —C(O)OR^{a}, —C(O)NR^{b}R^{c}, —C(NR^{a})NR^{b}R^{c}, —OR^{a}, OC(O)R^{a}, —OC(O)OR^{a}, —OC(O)NR^{b}R^{c}, —OC(═NR^{a})NR^{b}R^{c}, —OS(O)R^{a}, OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)R^{b}R^{c}, or —NR^{a}S(O)₂R^{b}R^{c}; wherein R^{a}, R^{b}, R^{c}, and R^{d} are each independently, e.g., hydrogen, alkyl, alkenyl, alkynyl, cycloalkyl, aryl, heteroaryl, or heterocyclyl, each optionally substituted, e.g., as described herein; or R^{b} and R^{c} together with the N atom to which they are attached form heterocyclyl or heteroaryl, each optionally substituted, e.g., as described herein. The group can be substituted with any described moiety, including, but not limited to, one or more moieties selected from the group consisting of halogen (fluoro, chloro, bromo, or iodo), hydroxyl, amino, alkylamino (e.g., monoalkylamino, dialkylamino, or trialkylamine), arylamino (e.g., monoarylamino, diarylamino, or triarylamino), alkoxy, aryloxy, nitro, cyano, sulfonic acid, sulfate, phosphonic acid, phosphate, or phosphonate, either unprotected or protected as necessary, as known to those skilled in the art, for example, as taught in Greene, et al., Protective Groups in Organic Synthesis, John Wiley and Sons, Second Edition, 1991. As used herein, all groups that can be substituted in one embodiment are “optionally substituted,” unless otherwise specified.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, or no less than about 94% no less than about 95%, no less than about 96%, no less than about 97%, no less than about 98%, no less than about 99%, or no less than about 99.5%, no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

### Compounds

HCV has a single positive-stranded RNA genome having about 9.6 kb in length that encodes a large polyprotein having about 3010 amino acids. This precursor polyprotein is then processed into a range of structural proteins, including a core protein, C, and envelope glycoproteins, E1 and E2; and non-structural proteins, including NS2, NS3, NS4A, NS4B, NS5A, and NS5B, by host signal peptidases and two viral proteases, NS2-3 and NS3. The NS3 protein contains a trypsin-like serine protease domain at its N-terminus, while its C-terminal domain has helicase activity. Because of its vital role in viral replication, HCV NS3 serine protease has been actively pursued as a drug target for developing a new anti-HCV therapy.

Inhibitors of HCV NS3 protease that have been reported include linear and cyclic peptides and peptide mimetics, and non-peptide molecules (Llinàs-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; U.S. Pat. App. Pub. Nos.: 2002/0016294, 2002/0016442; 2002/0037998; 2002/0032175; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781; WO 98/17679; WO 98/22496; WO 99/07734; WO 00/059929; WO 00/09543; WO 02/060926; WO 02/08187; WO 02/008251; WO 02/008256; WO 02/08198; WO 02/48116; WO 02/48157; WO 02/48172; WO 03/053349; WO 03/064416; WO 03/064456; WO 03/099274; WO 03/099316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; and WO 2007/056120). However, citation of any reference herein is not an admission that such reference is prior art to the present disclosure.

Provided herein are compounds which are useful for the treatment of HCV infection, which, in one embodiment, can have activity as HCV serine protease inhibitors. Also provided herein are pharmaceutical compositions that comprise the compounds, methods of the manufacture of the compounds, and methods of use of the compounds for the treatment of HCV infection in a host in need of such treatment.

In one embodiment, provided herein is a compound of Formula I:

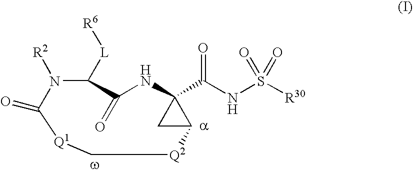

or a single enantiomer, a mixture of enantiomers, an individual diastereomer, or a mixture of diastereomers thereof; or a pharmaceutically acceptable salt, solvate, or prodrug thereof;
wherein:

R² is hydrogen, C₁₋₆ alkyl, C₃₋₇ cycloalkyl, C₆₋₁₄ aryl, heterocyclyl, or heteroaryl;

R⁶ is hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl;

R³⁰ is hydrogen, C₁₋₆ alkyl, C₂₋₆ alkenyl, C₂₋₆ alkynyl, C₃₋₇ cycloalkyl, C₆₋₁₄ aryl, heteroaryl, heterocyclyl, or C₁₋₆ alkyl-C₃₋₇ cycloalkylene;

L is a bond, C₁₋₆ alkylene, C₂₋₆ alkenylene, C₂₋₆ alkynylene, C₃₋₇ cycloalkyl, or —(CR^{a}R^{b})ₚX—; wherein p is an integer of 0, 1, 2, or 3; R^{a} and R^{b} are each independently hydrogen, halo, cyano, hydroxyl, or alkoxy; and X is —O—, —S—, —C(O)—, —C(O)O—, —OC(O)O—, —C(O)NR¹⁴—, —C(═NR¹⁴)NR¹⁵—, —NR¹⁴C(O)NR¹⁵—, —NR¹⁴C(═NR¹⁵)NR¹⁶—, —NR¹⁴S(O)ₖR¹⁵—, —NR¹⁴S(O)ₖNR¹⁵—, —S(O)ₖ—, —S(O)ₖNR¹⁴—, —P(O)OR¹⁴—, or —OP(O)OR¹⁴—; where R¹⁴, R¹⁵, and R¹⁶ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and each k is independently an integer of 1 or 2;

Q¹ is —O—, —N(R¹⁷)—, —C(R¹⁸R¹⁹)—, or —CR¹⁷(NR¹⁸R¹⁹)—; wherein:
R¹⁷ and R¹⁸ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and
R¹⁹ is hydrogen, C₁₋₆ alkyl, C₂₋₆ alkenyl, C₂₋₆ alkynyl, C₃₋₇ cycloalkyl, C₆₋₁₄ aryl, heterocyclyl, heteroaryl, —C(O)R²⁰, —C(O)OR²⁰, —C(O)NR²¹R²²—C(═NR²⁰)NR²¹R²², or S(O)ₘR²⁰; where R²⁰, R²¹, and R²² are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R²¹ and R²² are linked together with the N atom to which they are attached to form heterocyclyl or heteroaryl; and m is an integer of 0, 1, or 2; or
R¹⁸ and R¹⁹ are linked together with the C or N atom to which they are attached to form cycloalkyl, heterocyclyl, or heteroaryl; and

Q² is C₃₋₉ alkylene, C₃₋₉ alkenylene, or C₃₋₉ alkynylene, each optionally containing one to three heteroatoms in the chain of the alkylene, independently selected from O, N, and S;

wherein each alkyl, alkylene, alkenyl, alkenylene, alkynyl, alkynylene, cycloalkyl, cycloalkylene, aryl, heteroaryl, and heterocyclyl is independently, optionally substituted with one or more substituents Q, each Q independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)NR^{f}R^{g}, —OC(═NR^{e})NR^{f}R^{g}, —OS(O)R^{e}, —OS(O)₂R^{e}, —OS(O)NR^{f}R^{f}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} are linked together to form heterocyclyl, along with the N atom to which they are attached.

In another embodiment, provided herein is a compound of Formula II:

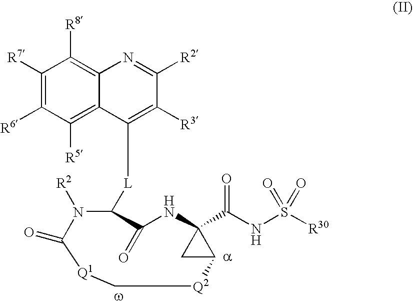

wherein:

R², R³⁰, L, Q¹, and Q² are each as defined herein; and

R^{2′}, R^{3′}, R^{5′}, R^{6′}, R^{7′}, and R^{8′} are each independently:
hydrogen, halo, cyano, trifluoromethyl, or nitro;
C₁₋₆ alkyl, C₂₋₆ alkenyl, C₂₋₆ alkynyl, C₃₋₇ cycloalkyl, C₆₋₁₄ aryl, heteroaryl, or heterocyclyl; or
—C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═O—NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, —S(O)₂R^{a}, or —S(O)₂NR^{b}R^{c}; wherein R^{a}, R^{b}, R^{c}, and R^{d} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{b} and R^{c} are linked together to form heterocyclyl or heteroaryl, along with the N atom to which they are attached;

wherein each alkyl, alkenyl, alkynyl, cycloalkyl, aryl, heteroaryl, and heterocyclyl is independently, optionally substituted with one or more substituents Q as described herein.

In certain embodiments, Q² is C₃₋₉ alkylene. In certain embodiments, Q² is C₃₋₉ alkenylene. In certain embodiments, Q² is C₃₋₉ alkenylene having one carbon-carbon double bond in either cis or trans configuration. In certain embodiments, Q² is C₃₋₉ alkenylene having one carbon-carbon double bond in cis configuration. In certain embodiments, Q² is C₃₋₉ alkenylene having one carbon-carbon double bond in trans configuration. In certain embodiments, Q² is C₃₋₉ alkynylene.

In certain embodiments, Q² is selected from the group consisting of:

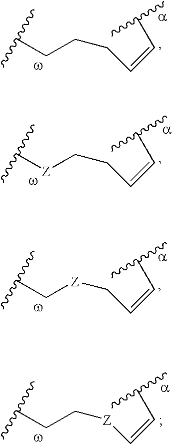

wherein:

Z is —O—, —S—, or —N(R^{Z})—, wherein R^{Z} is hydrogen, C₁₋₆ alkyl, aryl, heteroaryl, heterocyclyl, —C(O)R^{Za}, —C(O)OR^{Za}, —C(O)NR^{Zb}R^{Zc}, —S(O)₂NR^{Zb}R^{Zc}, or —S(O)₂R^{Za}; and

each R^{Za}, R^{Zb}, and R^{Zc} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or

R^{Zb} and R^{Zc} together with the N atom to which they are attached form heterocyclyl or heteroaryl;

wherein each alkyl, alkenyl, alkynyl, cycloalkyl, aryl, heteroaryl, and heterocyclyl is optionally substituted with one or more substituents Q as described herein.

In yet another embodiment, provided herein is a compound of Formula III:

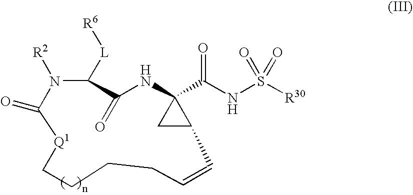

wherein:

R², R⁶, R³⁰, L, and Q¹ are each as defined herein; and

n is an integer ranging from 0 to 5.

In yet another embodiment, provided herein is a compound of Formula IV:

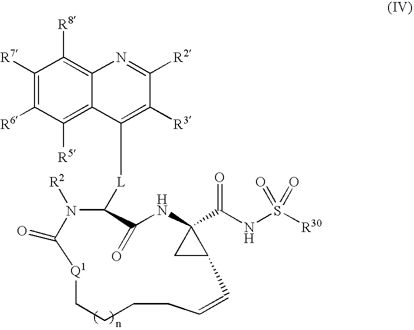

wherein R², R³⁰, R^{2′}, R^{3′}, R^{5′}, R^{6′}, R^{7′}, R^{8′}, L, Q¹, and n are each as defined herein.

In yet another embodiment, provided herein is a compound of Formula V:

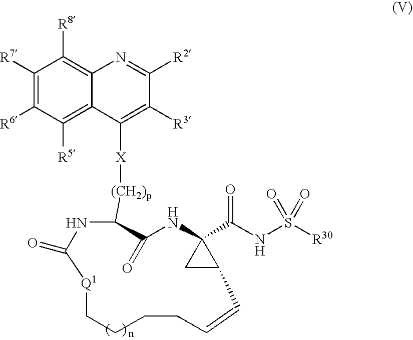

wherein R³⁰, R^{2′}, R^{3′}, R^{5′}, R^{6′}, R^{7′}, R^{8′}, Q¹, X, and n are each as defined herein.

In one embodiment, provided herein is a compound of Formula VIa:

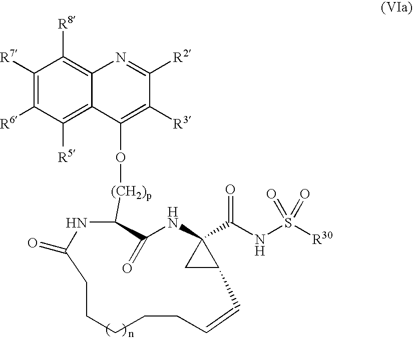

wherein R³⁰, R^{2′}, R^{3′}, R^{5′}, R^{6′}, R^{7′}, R^{8′}, n, and p are as defined herein.

In another embodiment, provided herein is a compound of Formula VIb:

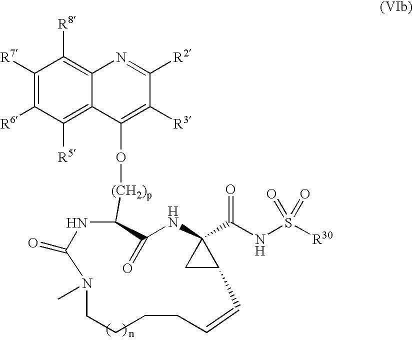

wherein R³⁰, R^{2′}, R^{3′}, R^{5′}, R^{6′}, R^{7′}, R^{8′}, n, and p are as defined herein.

In yet another embodiment, provided herein is a compound of Formula VIc:

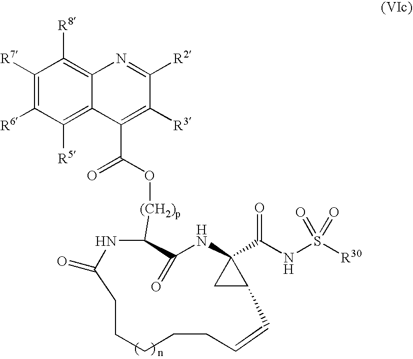

wherein R³⁰, R^{2′}, R^{3′}, R^{5′}, R^{6′}, R^{7′}, R^{8′}, n, and p are as defined herein.

In yet another embodiment, provided herein is a compound of Formula VId:

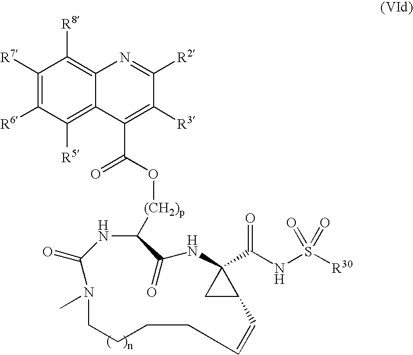

wherein R³⁰, R^{2′}, R^{3′}, R^{5′}, R^{6′}, R^{7′}, R^{8′}, n, and p are as defined herein.

In yet another embodiment, provided herein is a compound of Formula VIe:

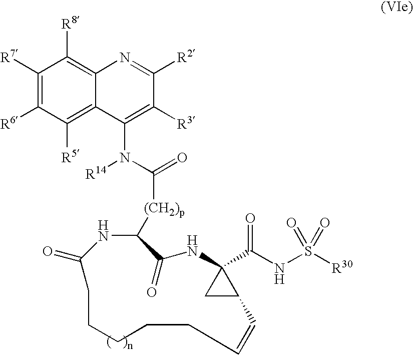

wherein R³⁰, R^{2′}, R^{3′}, R^{5′}, R^{6′}, R^{7′}, R^{8′}, n, and p are as defined herein.

In yet another embodiment, provided herein is a compound of Formula VIf:

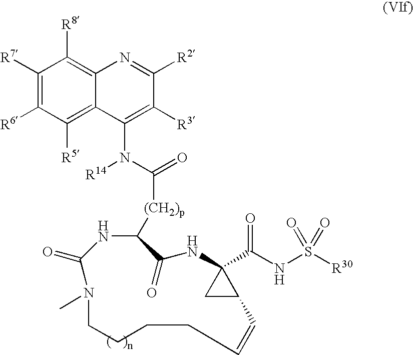

wherein R³⁰, R^{2′}, R^{3′}, R^{5′}, R^{6′}, R^{7′}, R^{8′}, n, and p are as defined herein.

In yet another embodiment, provided herein is a compound of Formula VIg:

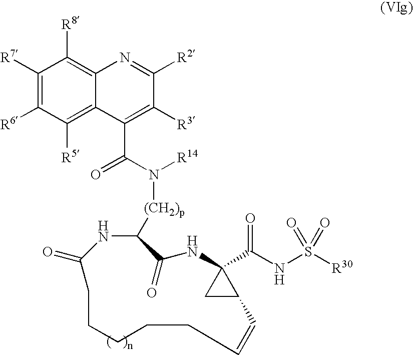

wherein R³⁰, R^{2′}, R^{3′}, R^{5′}, R^{6′}, R^{7′}, R^{8′}, n, and p are as defined herein.

In yet another embodiment, provided herein is a compound of Formula VIh:

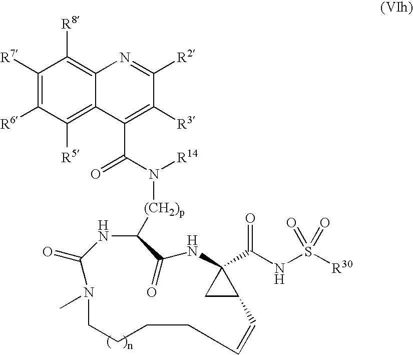

wherein R³⁰, R^{2′}, R^{3′}, R^{5′}, R^{6′}, R^{7′}, R^{8′}, n, and p are as defined herein.

The groups, R², R⁵, R⁶, R⁸, R³⁰, R^{2′}, R^{3′}, R^{5′}, R^{6′}, R^{7′}, R^{8′}, L, Q, X, k, m, n, and p in Formulae I, II, III, IV, V, VIa, VIb, VIc, VId, VIe, VIf, VIg, and VIh are further defined in the following embodiments, independently or in combination. All combinations of such embodiments are within the scope of this disclosure.

In certain embodiments, n is 0, 1, 2, 3, 4, or 5. In certain embodiments, n is 0. In certain embodiments, n is 1. In certain embodiments, n is 2. In certain embodiments, n is 3. In certain embodiments, n is 4. In certain embodiments, n is 5.

In certain embodiments, R⁶ is C₁₋₆ alkyl, C₃₋₇ cycloalkyl, C₆₋₁₄ aryl, heteroaryl, or heterocyclyl, each optionally substituted with one or more substituents Q as described herein. In certain embodiments, R⁶ is C₃₋₇ cycloalkyl, C₆₋₁₄ aryl, heteroaryl, or heterocyclyl, each optionally substituted with one or more substituents Q as described herein. In certain embodiments, R⁶ is C₃₋₇ cycloalkyl, optionally substituted with one or more substituents Q as described herein. In certain embodiments, R⁶ is C₆₋₁₄ aryl, optionally substituted with one or more substituents Q as described herein. In certain embodiments, R⁶ is heteroaryl, optionally substituted with one or more substituents Q as described herein. In certain embodiments, R⁶ is heterocyclyl, optionally substituted with one or more substituents Q as described herein.

In certain embodiments, wherein R⁶ is selected from the group consisting of:

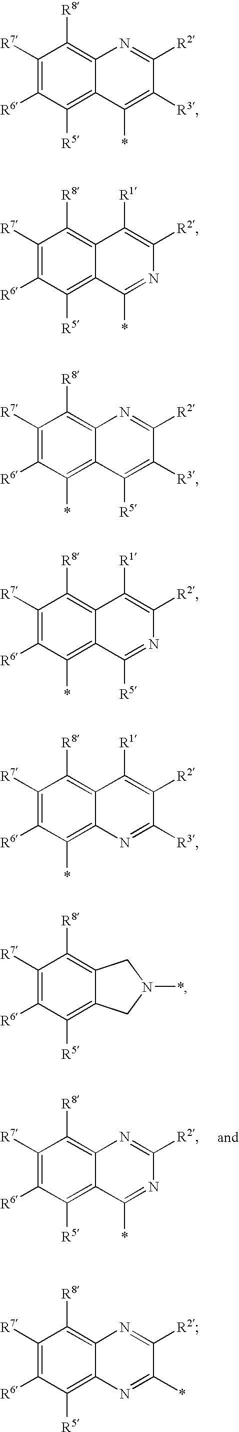

wherein R^{1′}, R^{2′}, R^{3′}, R^{5′}, R^{6′}, R^{7′}, and R^{8′} are each as defined herein.

In certain embodiments, R^{2′} is C₁₋₆ alkyl, C₂₋₆ alkenyl, C₂₋₆ alkynyl, C₃₋₇ cycloalkyl, C₆₋₁₄ aryl, heterocyclyl, or heteroaryl, each optionally substituted with one or more substituents Q as described herein. In certain embodiments, R^{2′} is C₆₋₁₄ aryl, heterocyclyl, or heteroaryl, each optionally substituted with one or more substituents Q as described herein.

In certain embodiments, R^{2′} is selected from the group consisting of:

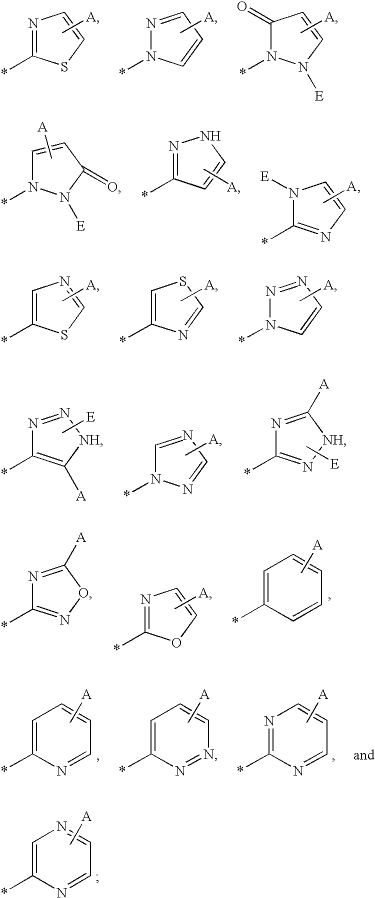

wherein

A is hydrogen, halo, cyano, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, NR^{a}S(O)₂NR^{b}R^{c}, —SR^{a}, —S(O)R^{a}, —S(O)₂R^{a}, or —S(O)₂NR^{b}R^{c};

E is hydrogen, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═O—NR^{a})NR^{b}R^{c}, OS(O)R^{a}, —OS(O)₂R^{b}, —OS(O)NR^{b}R^{c}, —OS(O)₂NR^{b}R^{c}, —NR^{b}R^{c}, —NR^{a}C(O)R^{b}, —NR^{a}C(O)OR^{b}, NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, —S(O)₂R^{a}, or —S(O)₂NR^{b}R^{c}; and

R^{a}, R^{b}, R^{c}, and R^{d} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{b} and R^{c} together with the N atom to which they are attached form heterocyclyl or heteroaryl;

wherein each alkyl, alkenyl, alkynyl, cycloalkyl, aryl, heteroaryl, and heterocyclyl is optionally substituted with one or more substituents Q as described herein.

In certain embodiments, A is hydrogen, halo, cyano, nitro, C₁₋₆ alkyl, C₂₋₆ alkenyl, C₂₋₆ alkynyl, C₃₋₇ cycloalkyl, C₆₋₁₄ aryl, heteroaryl, or heterocyclyl, wherein each alkyl, alkenyl, alkynyl, cycloalkyl, aryl, heteroaryl, and heterocyclyl is optionally substituted with one or more substituents Q as described herein.

In certain embodiments, A is hydrogen, C₁₋₆ alkyl, wherein alkyl is optionally substituted with one or more substituents Q as described herein. In certain embodiments, A is hydrogen. In certain embodiments, A is C₁₋₆ alkyl, optionally substituted with one or more substituents Q as described herein. In certain embodiments, A is C₂₋₆ alkenyl, optionally substituted with one or more substituents Q as described herein. In certain embodiments, A is C₂₋₆ alkynyl, optionally substituted with one or more substituents Q as described herein. In certain embodiments, A is C₃₋₇ cycloalkyl, optionally substituted with one or more substituents Q as described herein. In certain embodiments, A is C₆₋₁₄ aryl, optionally substituted with one or more substituents Q as described herein. In certain embodiments, A is heteroaryl, optionally substituted with one or more substituents Q as described herein. In certain embodiments, A is heterocyclyl, optionally substituted with one or more substituents Q as described herein. In certain embodiments, A is —OR^{a}, wherein R^{a} is as defined herein. In certain embodiments, A is —NR^{b}R^{c}, wherein R^{b} and R^{c} are each as defined herein.

In certain embodiments, A is hydrogen, fluoro, methyl, ethyl, n-propyl, isopropyl, cyclopropyl, isobutyl, isopentyl, trifluoromethyl, benzyl, 2-morpholin-4-yl-ethyl, cyclobutyl, ethynyl, methoxy, ethoxy, or isopropylamino. In certain embodiments, A is hydrogen, methyl, isopropyl, isobutyl, trifluoromethyl, cyclopropyl, cyclobutyl, ethynyl, methoxy, ethoxy, or isopropylamino.

In certain embodiments, E is hydrogen, C₁₋₆ alkyl, C₂₋₆ alkenyl, C₂₋₆ alkynyl, C₃₋₇ cycloalkyl, C₆₋₁₄ aryl, heterocyclyl, or heteroaryl, wherein each alkyl, alkenyl, alkynyl, cycloalkyl, aryl, heteroaryl, and heterocyclyl is optionally substituted with one or more substituents Q as described herein. In certain embodiments, E is hydrogen, methyl, ethyl, n-propyl, isopropyl, cyclopropyl, isobutyl, isopentyl, trifluoromethyl, benzyl, 2-morpholin-4-yl-ethyl, cyclobutyl, ethynyl, methoxy, ethoxy, or isopropylamino. In certain embodiments, E is hydrogen, methyl, ethyl, n-propyl, isopropyl, isobutyl, isopentyl, benzyl, or 2-morpholin-4-yl-ethyl.

In certain embodiments, R^{2′} is selected from the group consisting of:

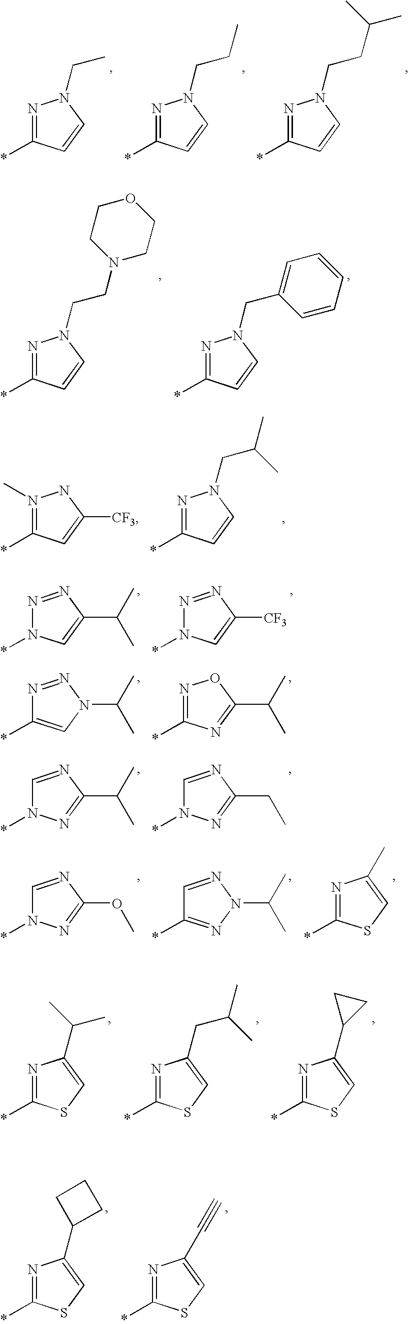

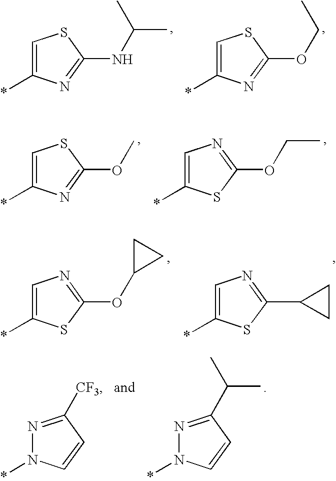

In certain embodiments, R^{3′} is hydrogen, hydroxyl, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, or —OR^{a}; and R^{a} is C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each alkyl, alkenyl, alkynyl, cycloalkyl, aryl, heteroaryl, and heterocyclyl is optionally substituted with one or more substituents Q as described herein.

In certain embodiments, R^{3′} is halo or —OR^{a}, wherein R^{a} is as defined herein. In certain embodiments, R^{3′} is —OR^{a}, wherein R^{a} is as defined herein. In certain embodiments, R^{a} is C₁₋₆ alkyl, C₃₋₇ cycloalkyl, or C₆₋₁₄ aryl, each optionally substituted as described herein. In certain embodiments, R^{a} is C₁₋₆ alkyl or C₃₋₇ cycloalkyl, each optionally substituted with one or more substituents Q as described herein. In certain embodiments, R^{3′} is hydrogen. In certain embodiments, R^{3′} is halo. In certain embodiments, R^{3′} is fluoro or chloro. In certain embodiments, R^{3′} is methoxy.

In certain embodiments, R^{5′} is hydrogen, hydroxyl, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, or —OR^{a}; and R^{a} is C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each alkyl, alkenyl, alkynyl, cycloalkyl, aryl, heteroaryl, and heterocyclyl is optionally substituted with one or more substituents Q as described herein.

In certain embodiments, R^{5′} is halo or —OR^{a}, wherein R^{a} is as defined herein. In certain embodiments, R^{5′} is —OR^{a}, wherein R^{a} is as defined herein. In certain embodiments, R^{a} is C₁₋₆ alkyl, C₃₋₇ cycloalkyl, or C₆₋₁₄ aryl, each optionally substituted as described herein. In certain embodiments, R^{a} is C₁₋₆ alkyl or C₃₋₇ cycloalkyl, each optionally substituted with one or more substituents Q as described herein. In certain embodiments, R^{5′} is hydrogen. In certain embodiments, R^{5′} is halo. In certain embodiments, R^{5′} is fluoro or chloro. In certain embodiments, R^{5′} is methoxy.

In certain embodiments, R^{6′} is hydrogen, hydroxyl, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, or —OR^{a}; and R^{a} is C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each alkyl, alkenyl, alkynyl, cycloalkyl, aryl, heteroaryl, and heterocyclyl is optionally substituted with one or more substituents Q as described herein.

In certain embodiments, R^{6′} is halo or —OR^{a}, wherein R^{a} is as defined herein. In certain embodiments, R^{6′} is —OR^{a}, wherein R^{a} is as defined herein. In certain embodiments, R^{a} is C₁₋₆ alkyl, C₃₋₇ cycloalkyl, or C₆₋₁₄ aryl, each optionally substituted as described herein. In certain embodiments, R^{a} is C₁₋₆ alkyl or C₃₋₇ cycloalkyl, each optionally substituted with one or more substituents Q as described herein. In certain embodiments, R^{6′} is hydrogen. In certain embodiments, R^{6′} is halo. In certain embodiments, R^{6′} is fluoro or chloro. In certain embodiments, R^{6′} is methoxy.

In certain embodiments, R^{7′} is hydrogen, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, —OR^{a}, —NR^{a}S(O)₂R^{b}, or —S(O)NR^{b}R^{c}, wherein each R^{a}, R^{b}, and R^{c} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

In certain embodiments, R^{7′} is hydrogen, hydroxyl, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, or —OR^{a}, and R^{a} is C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each alkyl, alkenyl, alkynyl, cycloalkyl, aryl, heteroaryl, and heterocyclyl is optionally substituted with one or more substituents Q as described herein. In certain embodiments, R^{7′} is C₁₋₆ alkyl, optionally substituted with one or more substituents Q. In certain embodiments, R^{7′} is difluoromethyl.

In certain embodiments, R^{7′} is halo or —OR^{a}, wherein R^{a} is as defined herein. In certain embodiments, R^{7′} is —OR^{a}, wherein R^{a} is as defined herein. In certain embodiments, R^{a} is C₁₋₆ alkyl, C₃₋₇ cycloalkyl, or C₆₋₁₄ aryl, each optionally substituted with one or more substituents Q as described herein. In certain embodiments, R^{a} is C₁₋₆ alkyl or C₃₋₇ cycloalkyl, each optionally substituted with one or more substituents Q as described herein. In certain embodiments, R^{7′} is methoxy. In certain embodiments, R^{7′} is halo. In certain embodiments, R^{7′} is fluoro or chloro. In certain embodiments, R^{7′} is hydrogen.

In certain embodiments, R^{6′} is —OR^{a} and R^{7′} is hydrogen, wherein R^{a} is as defined herein. In certain embodiments, R^{6′} is methoxy and R^{7′} is hydrogen.

In certain embodiments, R^{6′} is hydrogen and R^{7′} is —OR^{a}, wherein R^{a} is as defined herein. In certain embodiments, R^{6′} is hydrogen and R^{7′} is methoxy.

In certain embodiments, R^{8′} is hydrogen, hydroxyl, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, or —OR^{a}; and R^{a} is C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each alkyl, alkenyl, alkynyl, cycloalkyl, aryl, heteroaryl, and heterocyclyl is optionally substituted with one or more substituents Q as described herein.

In certain embodiments, R^{8′} is hydrogen, halo, or C₁₋₆ alkyl, optionally substituted with one or more substituents Q as described herein. In certain embodiments, R^{8′} is hydrogen. In certain embodiments, R^{8′} is halo. In certain embodiments, R^{8′} is hydrogen, fluoro, chloro, bromo, or methyl. In certain embodiments, R^{8′} is hydrogen. In certain embodiments, R^{8′} is fluoro, chloro, bromo, or iodo. In certain embodiments, R^{8′} is fluoro. In certain embodiments, R^{8′} is chloro. In certain embodiments, R^{8′} is bromo. In certain embodiments, R^{8′} is iodo. In certain embodiments, R^{8′} is C₁₋₆ alkyl, optionally substituted with one or more substituents Q as described herein. In certain embodiments, R^{8′} is methyl.

In certain embodiments, R^{8′} is —OR^{a}, where R^{a} is as defined herein. In certain embodiments, R^{8′} is —OR^{a}, where R^{a} is C₁₋₆ alkyl, optionally substituted with one or more substituents Q as described herein. In certain embodiments, R^{8′} is methoxy.

In certain embodiments, L is a bond. In certain embodiments, L is C₁₋₆ alkylene, optionally substituted with one or more substituents Q as described herein. In certain embodiments, L is mono or dihalo substituted C₁₋₆ alkylene. In certain embodiments, L is difluoro substituted C₁₋₆ alkylene. In certain embodiments, L is C₂₋₆ alkenylene, optionally substituted with one or more substituents Q as described herein. In certain embodiments, L is C₂₋₆ alkynylene, optionally substituted with one or more substituents Q as described herein. In certain embodiments, L is C₃₋₇ cycloalkylene, optionally substituted with one or more substituents Q as described herein.

In certain embodiments, L is —(CR^{a}R^{b})ₚX—, wherein R^{a}, R^{b}, X, and p are each as defined herein. In certain embodiments, L is —(CR^{a}R^{b})ₚO—, wherein R^{a}, R^{b}, and p are each as defined herein. In certain embodiments, L is —(CR^{a}R^{b})ₚC(O)—, wherein R^{a}, R^{b}, and p are each as defined herein. In certain embodiments, L is —C(O)O—, wherein R^{a}, R^{b}, and p are each as defined herein. In certain embodiments, L is —(CR^{a}R^{b})ₚOC(O)—, wherein R^{a}, R^{b}, and p are each as defined herein. In certain embodiments, L is —(CR^{a}R^{b})ₚOC(O)O—, wherein R^{a}, R^{b}, and p are each as defined herein. In certain embodiments, L is —(CR^{a}R^{b})ₚC(O)NR¹⁴—, wherein R^{a}, R^{b}, R¹⁴, and p are each as defined herein. In certain embodiments, L is —(CR^{a}R^{b})ₚNR¹⁴C(O)—, wherein R^{a}, R^{b}, R¹⁴, and p are each as defined herein. In certain embodiments, L is —(CR^{a}R^{b})ₚNR¹⁴C(O)NR¹⁵—, wherein R^{a}, R^{b}, R¹⁴, R¹⁵, and p are each as defined herein. In certain embodiments, L is —(CR^{a}R^{b})ₚC(═NR¹⁴)NR¹⁵—, wherein R^{a}, R^{b}, R¹⁴, R¹⁵, and p are each as defined herein. In certain embodiments, L is —(CR^{a}R^{b})ₚNR¹⁴C(═NR¹⁵)—, wherein R^{a}, R^{b}, R¹⁴, R¹⁵, and p are each as defined herein. In certain embodiments, L is —(CR^{a}R^{b})ₚNR¹⁴C(═NR¹⁵)NR¹⁶—, wherein R^{a}, R^{b}, R¹⁴, R¹⁵, R¹⁶, and p are each as defined herein. In certain embodiments, L is —(CR^{a}R^{b})ₚS(O)ₖ—, wherein R^{a}, R^{b}, k, and p are each as defined herein. In certain embodiments, L is —(CR^{a}R^{b})ₚS(O)ₖNR¹⁴—, wherein R^{a}, R^{b}, R¹⁴, k, and p are each as defined herein. In certain embodiments, L is wherein R^{a}, R^{b}, R¹⁴, k, and p are each as defined herein. In certain embodiments, L is —(CR^{a}R^{b})ₚNR¹⁴S(O)ₖ—, wherein R^{a}, R^{b}, R¹⁴, k, and p are each as defined herein. In certain embodiments, L is —(CR^{a}R^{b})ₚNR¹⁴S(O)ₖNR¹⁵—, wherein R^{a}, R^{b}, R¹⁴, R¹⁵, k, and p are each as defined herein. In certain embodiments, L is —(CR^{a}R^{b})ₚP(O)OR¹⁴—, wherein R^{a}, R^{b}, R¹⁴, k, and p are each as defined herein. In certain embodiments, L is —(CR^{a}R^{b})ₚOP(O)OR¹⁴—, wherein R^{a}, R^{b}, R¹⁴, k, and p are each as defined herein.

In certain embodiments, R^{a} and R^{b} are each independently hydrogen or halo. In certain embodiments, R^{a} and R^{b} are each independently hydrogen or fluoro. In certain embodiments, L is —(CH₂)ₚ—, wherein p is as defined herein. In certain embodiments, L is —CH₂—. In certain embodiments, L is —CH₂CH₂—. In certain embodiments, L is —CH₂CH₂CH₂—. In certain embodiments, L is —(CH₂)ₚCF₂—, wherein p is as defined herein. In certain embodiments, L is —CF₂—. In certain embodiments, L is —(CH₂)ₚO—, wherein p is as defined herein. In certain embodiments, L is —(CH₂)ₚC(O)—, wherein p is as defined herein. In certain embodiments, L is —(CH₂)ₚC(O)O—, wherein p is as defined herein. In certain embodiments, L is —(CH₂)ₚOC(O)—, wherein p is as defined herein. In certain embodiments, L is —(CH₂)ₚC(O)NR¹⁴—, wherein R¹⁴ and p is as defined herein. In certain embodiments, L is —(CH₂)ₚNR¹⁴C(O)—, wherein R¹⁴ and p is as defined herein. In certain embodiments, L is —(CH₂)ₚNR¹⁴C(O)NR¹⁵—, wherein R¹⁴, R¹⁵, and p are as defined herein.

In certain embodiments, R¹⁴ and R¹⁵ are each independently hydrogen, C₁₋₆ alkyl, C₃₋₇ cycloalkyl, C₆₋₁₄ aryl, heteroaryl, or heterocyclyl, wherein each alkyl, alkenyl, alkynyl, cycloalkyl, aryl, heteroaryl, and heterocyclyl is optionally substituted with one or more substituents Q as described herein. In certain embodiments, R¹⁴ and R¹⁵ are each independently hydrogen, C₁₋₆ alkyl, or C₃₋₇ cycloalkyl, wherein alkyl and cycloalkyl are each optionally substituted with one or more substituents Q as described herein. In certain embodiments, R¹⁴ and R¹⁵ are hydrogen.

In certain embodiments, p is 0. In certain embodiments, p is 1. In certain embodiments, p is 2. In certain embodiments, p is 3.

In certain embodiments, Q¹ is —O—.

In certain embodiments, Q¹ is —N(R¹⁷)—, wherein R¹⁷ is as defined herein. In one embodiment, R¹⁷ is hydrogen, C₁₋₆ alkyl, C₂₋₆ alkenyl, C₂₋₆ alkynyl, C₃₋₇ cycloalkyl, C₆₋₁₄ aryl, heterocyclyl, or heteroaryl, wherein each alkyl, alkenyl, alkynyl, cycloalkyl, aryl, heteroaryl, and heterocyclyl is optionally substituted with one or more substituents Q as described herein. In another embodiment, R¹⁷ is hydrogen, C₁₋₆ alkyl, or C₃₋₇ cycloalkyl, wherein alkyl and cycloalkyl are each optionally substituted with one or more substituents Q as described herein. In yet another embodiment, R¹⁷ is hydrogen or C₁₋₆ alkyl, optionally substituted with one or more substituents Q as described herein. In yet another embodiment, R¹⁷ is hydrogen. In yet another embodiment, R¹⁷ is C₁₋₆ alkyl, optionally substituted with one or more substituents Q as described herein. In still another embodiment, R¹⁷ is methyl.

In certain embodiments, Q¹ is —C(R¹⁸R¹⁹)—, wherein R¹⁸ and R¹⁹ are each as defined herein. In one embodiment, R¹⁸ and R¹⁹ are each independently hydrogen, C₁₋₆ alkyl, or C₃₋₇ cycloalkyl, wherein alkyl and cycloalkyl are each optionally substituted with one or more substituents Q as described herein. In another embodiment, R¹⁸ is hydrogen. In yet another embodiment, R¹⁹ is hydrogen. In yet another embodiment, R¹⁸ and R¹⁹ are hydrogen. In another embodiment, R¹⁸ is C₁₋₆ alkyl, optionally substituted with one or more substituents Q as described herein. In yet another embodiment, R¹⁹ is C₁₋₆ alkyl, optionally substituted with one or more substituents Q as described herein. In still another embodiment, R¹⁸ and R¹⁹ are each independently C₁₋₆ alkyl, optionally substituted with one or more substituents Q as described herein.

In certain embodiments, Q¹ is —C(R¹⁸R¹⁹)—, wherein R¹⁸ and R¹⁹ together with the C atom to which they are attached form cycloalkyl, optionally substituted with one or more substituents Q as described herein.

In certain embodiments, Q¹ is —CR¹⁷(NR¹⁸R¹⁹)—, wherein R¹⁷, R¹⁸, and R¹⁹ are each as defined herein. In one embodiment, R¹⁷ and R¹⁸ are each independently hydrogen, C₁₋₆ alkyl, or C₃₋₇ cycloalkyl, wherein alkyl and cycloalkyl are each optionally substituted with one or more substituents Q as described herein. In another embodiment, R¹⁷ is hydrogen or C₁₋₆ alkyl, optionally substituted with one or more substituents Q as described herein. In yet another embodiment, R¹⁷ is hydrogen. In yet another embodiment, R¹⁷ is C₁₋₆ alkyl, optionally substituted with one or more substituents Q as described herein. In yet another embodiment, R¹⁷ is methyl. In one embodiment, R¹⁸ is hydrogen or C₁₋₆ alkyl, optionally substituted with one or more substituents Q as described herein. In yet another embodiment, R¹⁸ is hydrogen. In yet another embodiment, R¹⁸ is C₁₋₆ alkyl, optionally substituted with one or more substituents Q as described herein. In yet another embodiment, R¹⁸ is methyl. In yet another embodiment, R¹⁷ and ¹⁸ are hydrogen.

In certain embodiments, R¹⁹ is hydrogen, —C(O)R²⁰, —C(O)OR²⁰, —C(O)NR²¹R²², or —C(═NR²⁰)NR²¹R²², wherein R²⁰, R²¹, and R²² are each as defined herein. In certain embodiments, R¹⁹ is hydrogen. In certain embodiments, R¹⁹ is —C(O)R²⁰, wherein R²⁰ is as defined herein. In certain embodiments, R¹⁹ is —C(O)NR²¹R²², wherein R²¹ and R²² are each as defined herein. In certain embodiments, R¹⁹ is —C(═O—NR²⁰)NR²¹R²², wherein R²⁰R²¹, and R²² are each as defined herein. In certain embodiments, R²¹ and R²² together with the N atom to which they are attached form heterocyclyl or heteroaryl, each optionally substituted with one or more substituents Q as described herein.

In certain embodiments, R¹⁹ is —C(O)OR²⁰, wherein R²⁰ is defined herein. In one embodiment, R²⁰ is C₁₋₆ alkyl, C₃₋₇ cycloalkyl, C₆₋₁₄ aryl, heterocyclyl, or heteroaryl, each optionally substituted with one or more substituents Q as described herein. In yet another embodiment, R²⁰ is C₁₋₆ alkyl, optionally substituted with one or more substituents Q as described herein. In yet another embodiment, R²⁰ is t-butyl. In yet another embodiment, R²⁰ is C₆₋₁₄ aryl, optionally substituted with one or more substituents Q as described herein. In still another embodiment, R²⁰ is benzyl.

In certain embodiments, R¹⁸ and R¹⁹ together with the N atom to which they are attached form heterocyclyl or heteroaryl, each optionally substituted with one or more substituents Q as described herein.

In certain embodiments, R³⁰ is C₁₋₆ alkyl, C₃₋₇ cycloalkyl, C₆₋₁₄ aryl, heteroaryl, heterocyclyl, or C₁₋₆ alkyl-C₃₋₇ cycloalkylene, each optionally substituted with one or more substituents Q as described herein. In certain embodiments, R³⁰ is C₁₋₆ alkyl, optionally substituted with one or more substituents Q as described herein. In certain embodiments, R³⁰ is C₃₋₇ cycloalkyl, optionally substituted as described herein. In certain embodiments, R³⁰ is cyclopropyl, 1-methylcyclopropyl, cyclobutyl, cyclopentyl, or cyclohexyl. In certain embodiments, R³⁰ is C₆₋₁₄ aryl, optionally substituted with one or more substituents Q as described herein. In certain embodiments, R³⁰ is heteroaryl, optionally substituted with one or more substituents Q as described herein. In certain embodiments, R³⁰ is heterocyclyl, optionally substituted with one or more substituents Q as described herein.

In certain embodiments, R³⁰ has the structure of

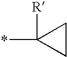

wherein R′ is hydrogen, halo, cyano, C₁₋₆ alkyl, C₂₋₆ alkenyl, C₂₋₆ alkynyl, C₆₋₁₄ aryl, C₃₋₇ cycloalkyl, heteroaryl, or heterocyclyl, wherein each alkyl, alkenyl, alkynyl, cycloalkyl, aryl, heteroaryl, and heterocyclyl is optionally substituted with one or more substituents Q as described herein.

In certain embodiments, R′ is hydrogen, halo, cyano, C₁₋₆ alkyl, or C₂₋₆ alkynyl, wherein each alkyl, alkynyl, and cycloalkyl is optionally substituted with one or more substituents Q as described herein. In certain embodiments, R′ is hydrogen. In certain embodiments, R′ is halo. In certain embodiments, R′ is fluoro, chloro, bromo, or iodo. In certain embodiments, R′ is fluoro. In certain embodiments, R′ is chloro. In certain embodiments, R′ is bromo. In certain embodiments, R′ is iodo. In certain embodiments, R′ is cyano. In certain embodiments, R′ is C₁₋₆ alkyl, optionally substituted with one or more substituents Q as described herein. In certain embodiments, R′ is C₂₋₆ alkynyl, optionally substituted with one or more substituents Q as described herein. In certain embodiments, R′ is hydrogen, fluoro, iodo, cyano, methyl, ethyl, trifluoromethyl, ethynyl, cyclopropylmethyl, or hydroxymethyl.

In certain embodiments, R² is hydrogen or C₁₋₆ alkyl, optionally substituted with one or more substituents Q as described herein. In certain embodiments, R² is hydrogen.

In certain embodiments, k is 1. In certain embodiments, k is 2.

In certain embodiments, m is 0. In certain embodiments, m is 1. In certain embodiments, m is 2.

In one embodiment, provided herein is a compound of Formula VII:

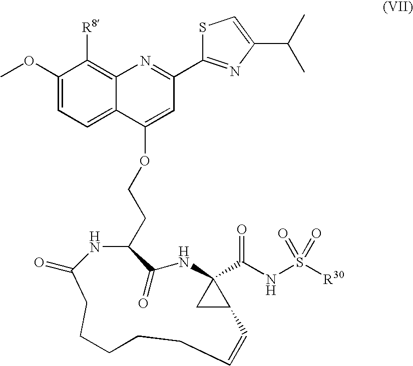

wherein R³⁰ and R^{8′} are each as defined herein.

In one embodiment, R³⁰ and R^{8′} in the compound of Formula VII are selected as a group from Table 1.

In another embodiment, provided herein is a compound of Formula VIII:

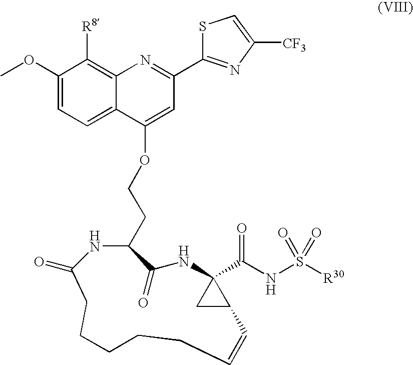

wherein R³⁰ and R^{8′} are each as defined herein.

In one embodiment, R³⁰ and R^{8′} in the compound of Formula VIII are selected as a group from Table 1.

TABLE 1

| R^{8′} | R³⁰ |
|---|---|
| H | Cyclopropyl |
| H | 1-Methylcyclopropyl |
| H | Cyclobutyl |
| H | Cyclopentyl |
| H | Cyclohexyl |
| H | Aminomethyl |
| Methyl | Cyclopropyl |
| Methyl | 1-Methylcyclopropyl |
| Methyl | Cyclobutyl |
| Methyl | Cyclopentyl |
| Methyl | Cyclohexyl |
| Methyl | Aminomethyl |
| Cl | Cyclopropyl |
| Cl | 1-Methylcyclopropyl |
| Cl | Cyclobutyl |
| Cl | Cyclopentyl |
| Cl | Cyclohexyl |
| Cl | Aminomethyl |
| F | Cyclopropyl |
| F | 1-Methylcyclopropyl |
| F | Cyclobutyl |
| F | Cyclopentyl |
| F | Cyclohexyl |
| F | Aminomethyl |
| Br | Cyclopropyl |
| Br | 1-Methylcyclopropyl |
| Br | Cyclobutyl |
| Br | Cyclopentyl |
| Br | Cyclohexyl |
| Br | Aminomethyl |

In yet another embodiment, provided herein is a compound of Formula IX:

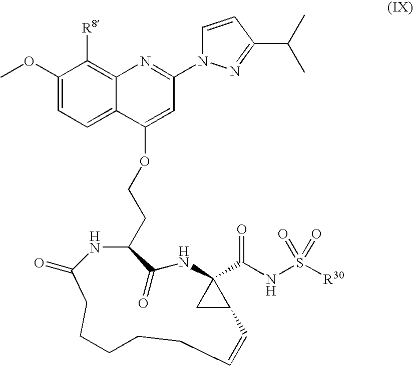

wherein R³⁰ and R^{8′} are each as defined herein.

In one embodiment, R³⁰ and R^{8′} in the compound of Formula IX are selected as a group from Table 1.

In yet another embodiment, provided herein is a compound of Formula X:

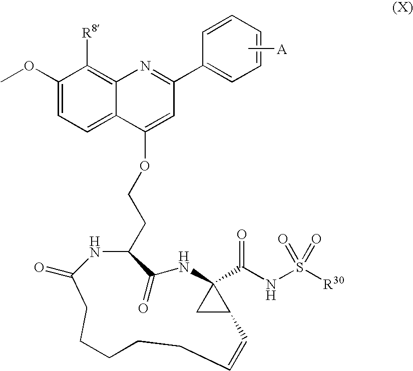

wherein R³⁰ and R^{8′} are each as defined herein, and A is hydrogen or fluoro.

In one embodiment, R³⁰ and R^{8′} in the compound of Formula X are selected as a group from Table 1.

In yet another embodiment, provided herein is a compound of Formula XI:

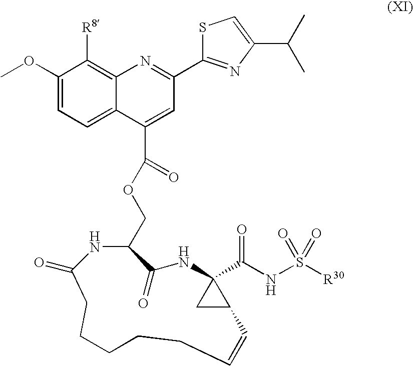

wherein R³⁰ and R^{8′} are each as defined herein.

In one embodiment, R³⁰ and R^{8′} in the compound of Formula XI are selected as a group from Table 1.

In another embodiment, provided herein is a compound of Formula XII:

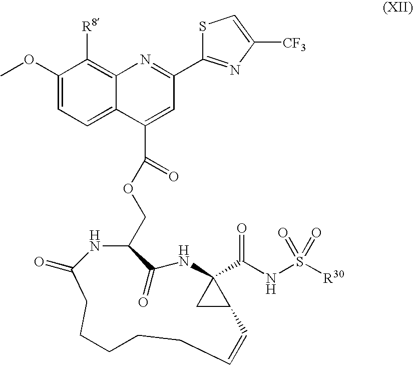

wherein R³⁰ and R^{8′} are each as defined herein.

In one embodiment, R³⁰ and R^{8′} in the compound of Formula XII are selected as a group from Table 1.

In yet another embodiment, provided herein is a compound of Formula XIII:

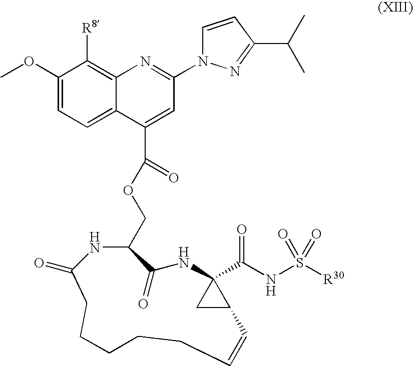

wherein R³⁰ and R^{8′} are each as defined herein.

In one embodiment, R³⁰ and R^{8′} in the compound of Formula XIII are selected as a group from Table 1.

In yet another embodiment, provided herein is a compound of Formula XIV:

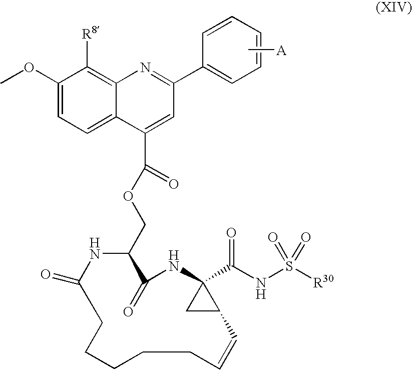

wherein R³⁰ and R^{8′} are each as defined herein, and A is hydrogen or fluoro.

In one embodiment, R³⁰ and R^{8′} in the compound of Formula XIV are selected as a group from Table 1.

In one embodiment, a compound of Formula IX is provided:

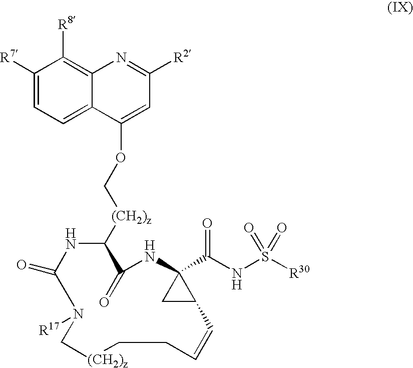

wherein:

R¹⁷ is hydrogen, methyl, or a peptidyl or mimetic;

R³⁰ is alkyl, cycloalkyl, or beta-amino alkyl;

R^{2′} is hydrogen, methyl, methoxy, methylsulfonyl, aryl, or heteroaryl;

R^{7′} is hydrogen, methyl, methoxy, or methylsulfonyl;

R^{8′} is hydrogen, halo, or methyl; and

each z is independently an integer of 0, 1, or 2.

In another embodiment, a compound of Formula X is provided:

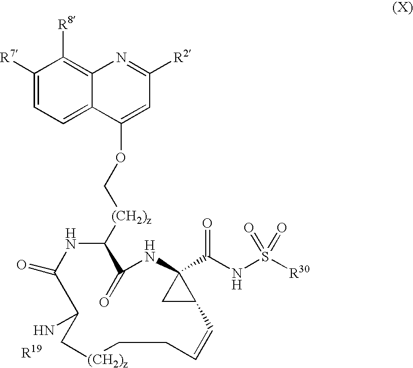

wherein:

R¹⁹ is hydrogen, methyl, or a peptidyl or mimetic;

R³⁰ is alkyl, cycloalkyl, or beta-amino alkyl;

R^{2′} is hydrogen, methyl, methoxy, methylsulfonyl, aryl, or heteroaryl;

R^{7′} is hydrogen, methyl, methoxy, or methylsulfonyl;

R^{8′} is hydrogen, halo, or methyl; and

each z is independently an integer of 0, 1, or 2.

In yet another embodiment, provided herein is

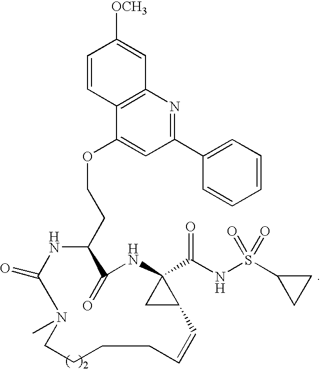

In one embodiment, provided herein is a compound selected from Table 2.

TABLE 2

| | |
|---|---|
| 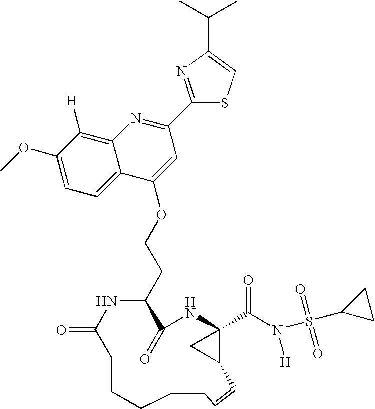 | 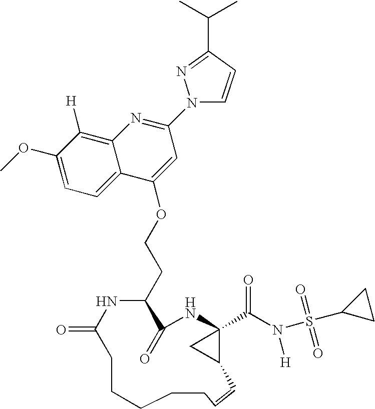 |
| 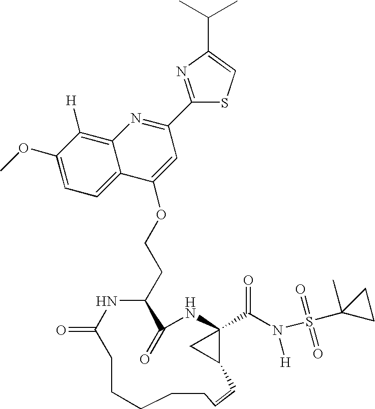 | 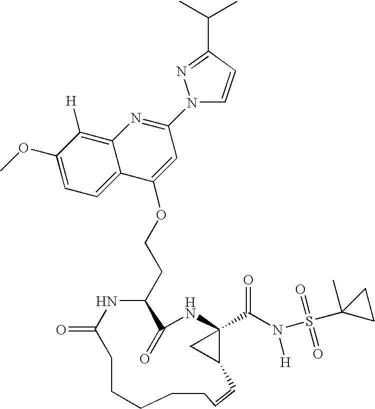 |
| 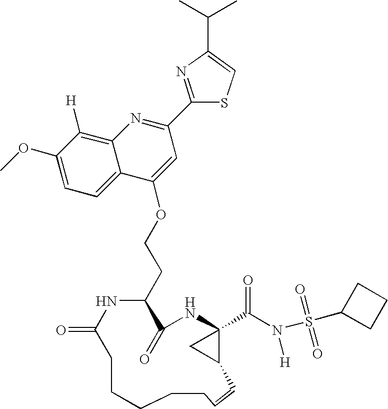 | 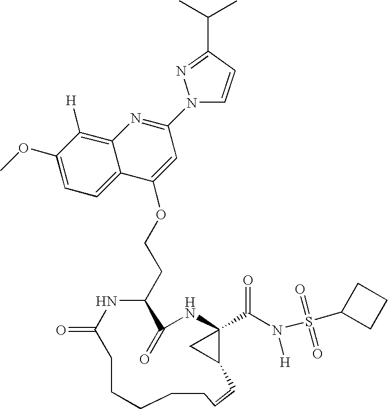 |
| 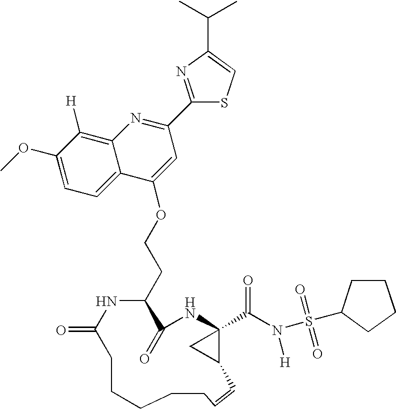 | 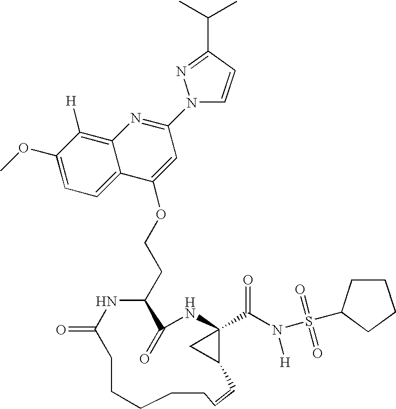 |
| 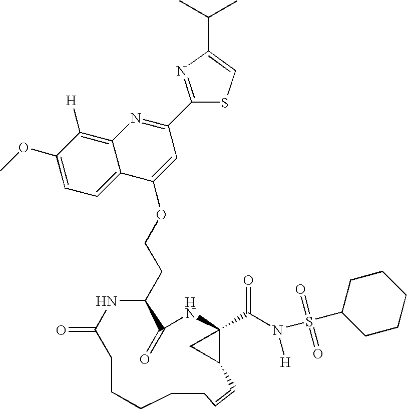 | 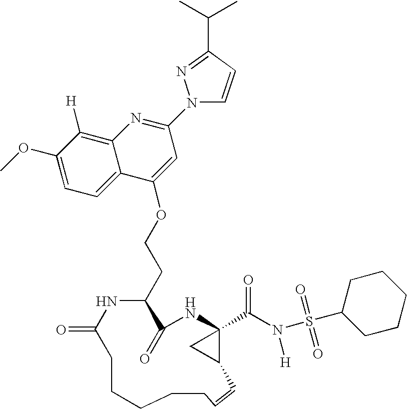 |
| 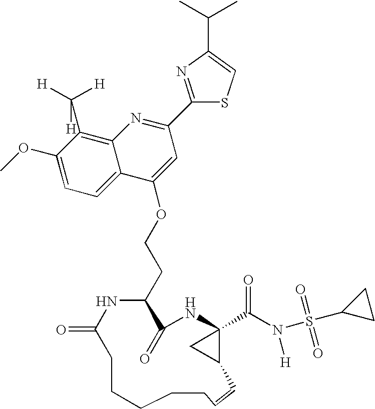 | 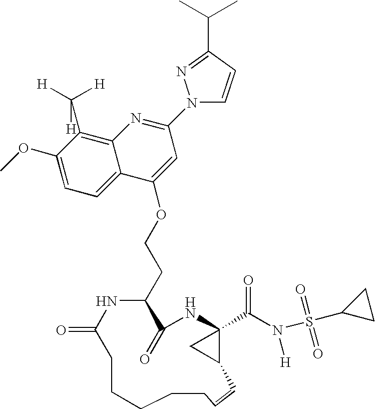 |
| 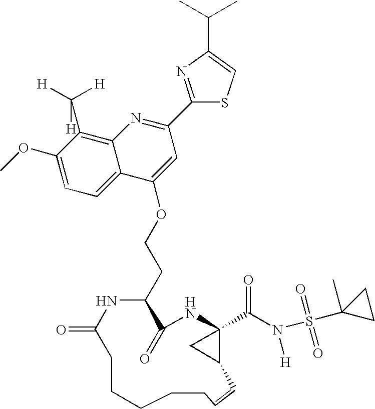 | 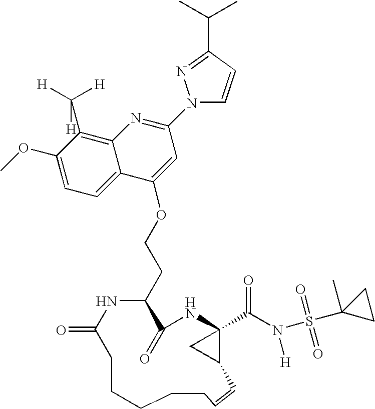 |
| 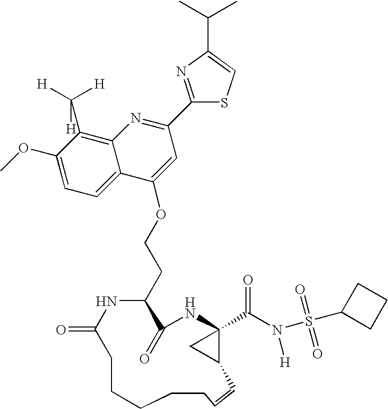 | 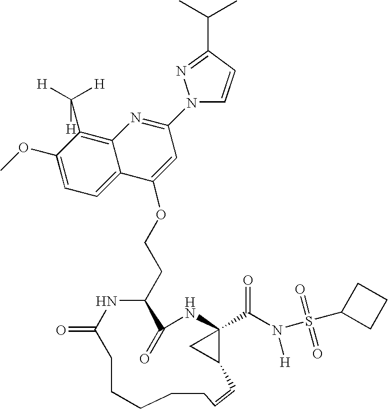 |
| 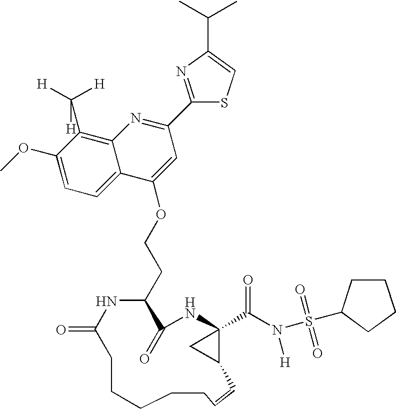 | 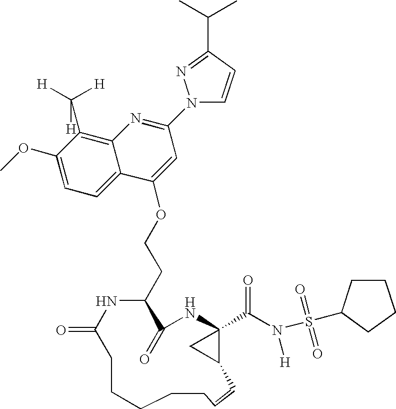 |
| 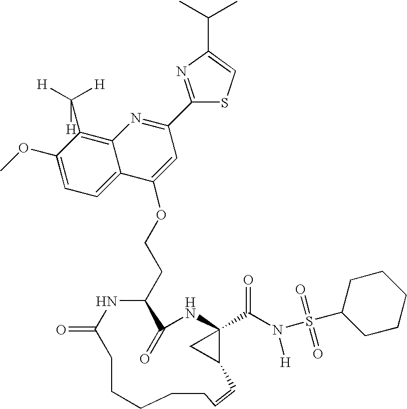 | 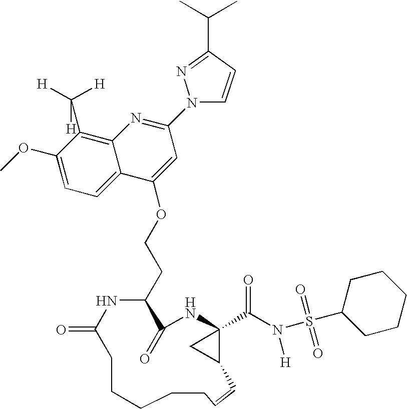 |
| 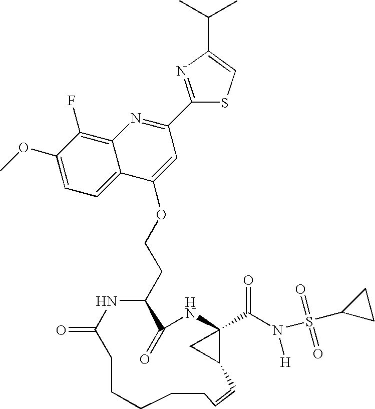 | 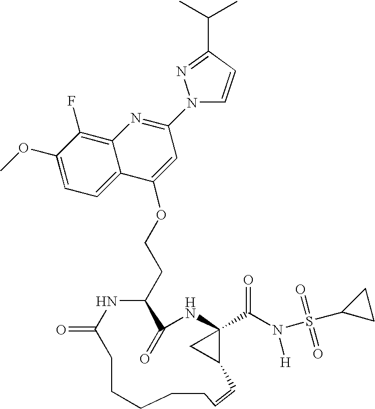 |
| 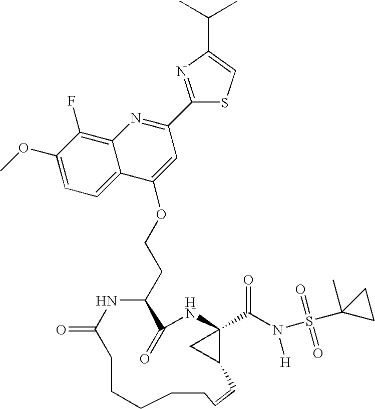 | 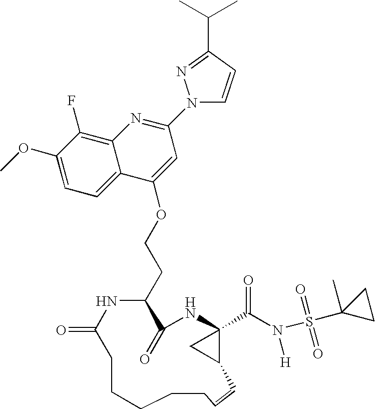 |
| 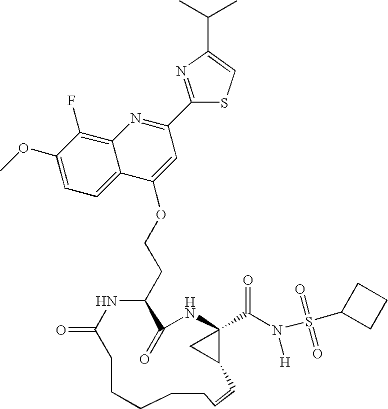 | 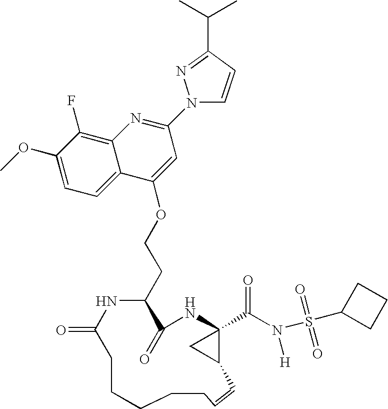 |
| 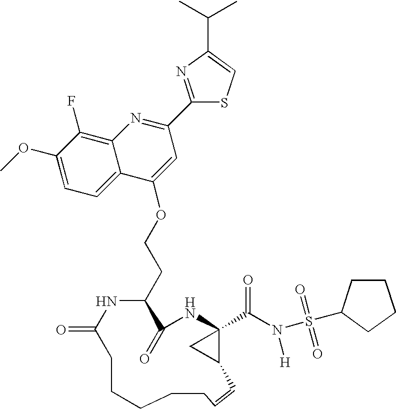 | 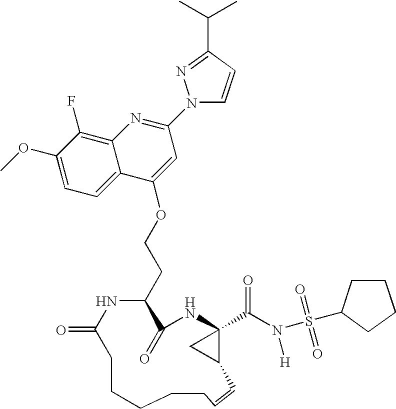 |
| 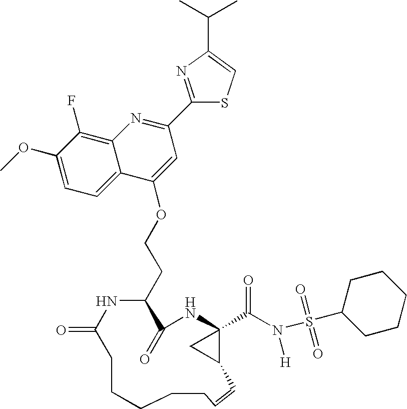 | 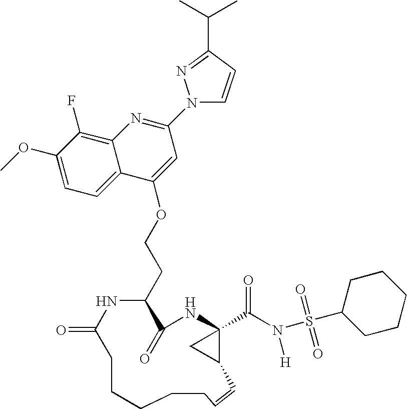 |

In another embodiment, provided herein is a compound selected from selected from the group consisting of:

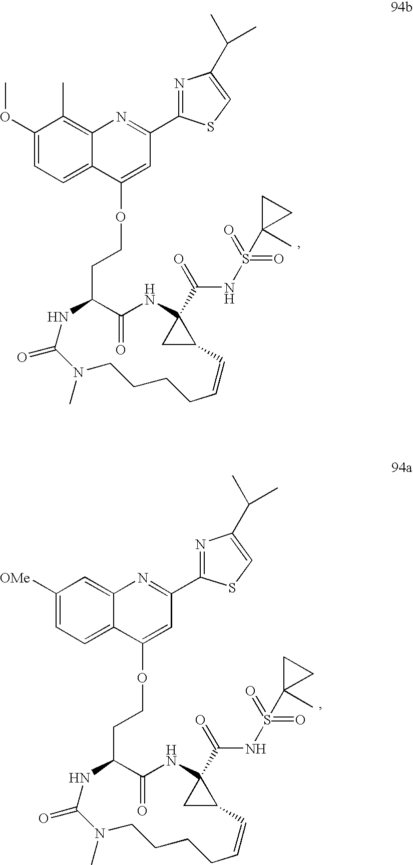

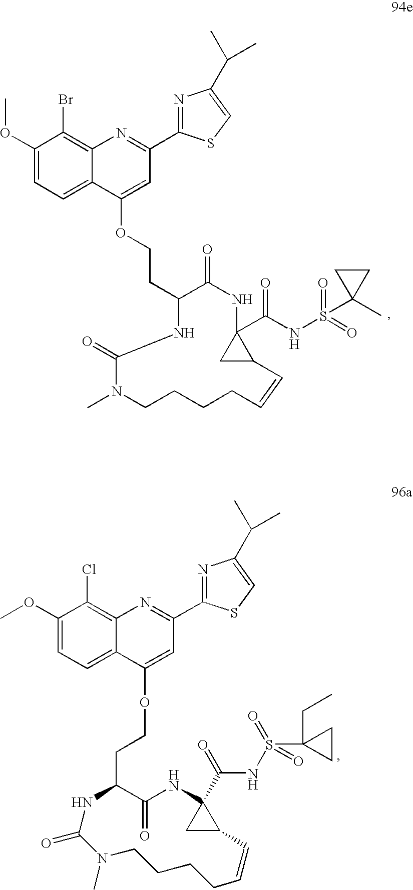

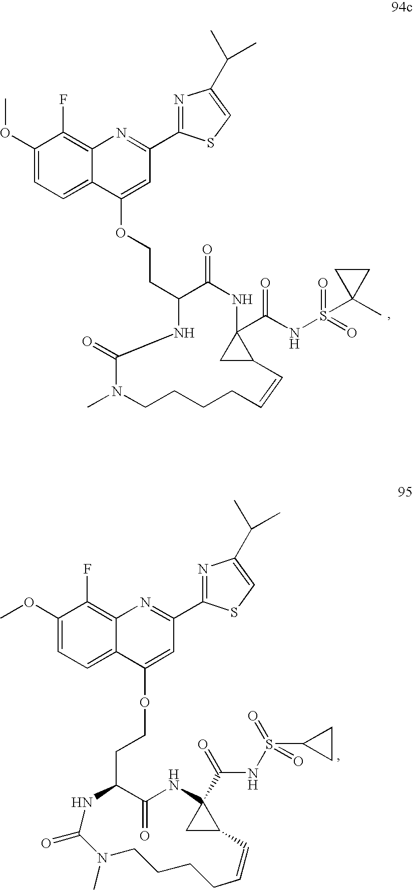

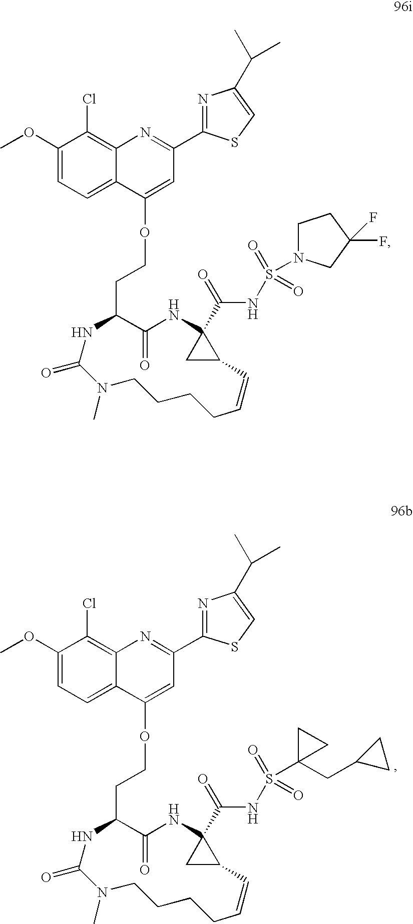

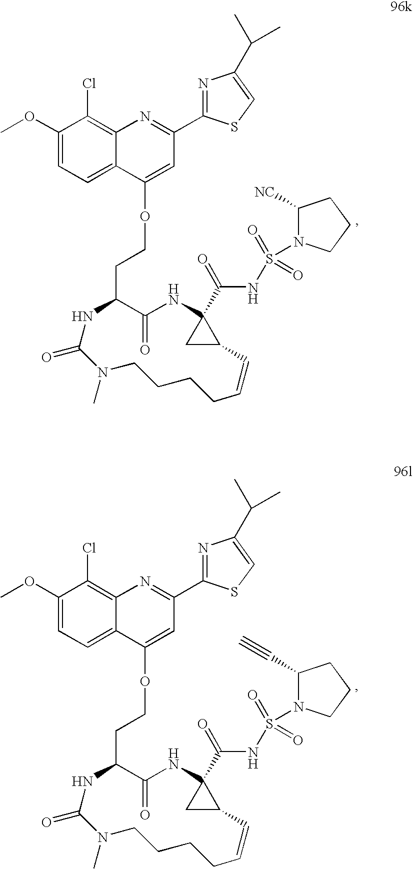

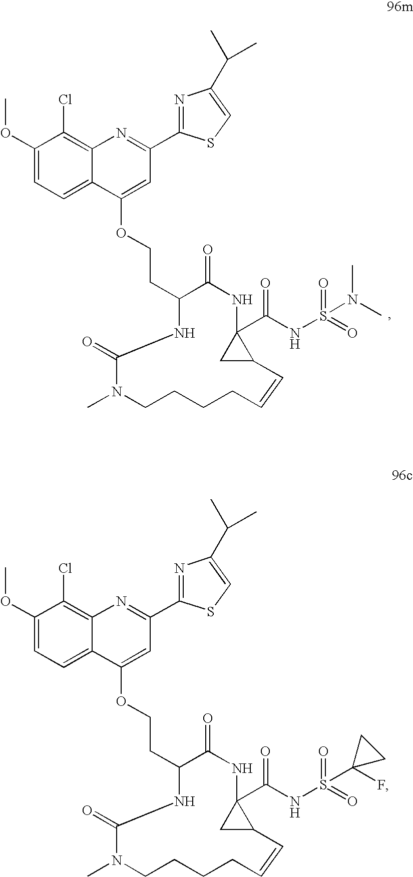

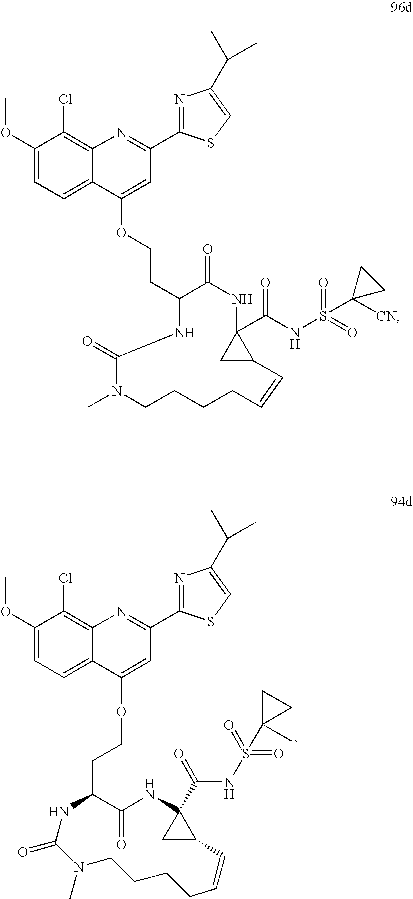

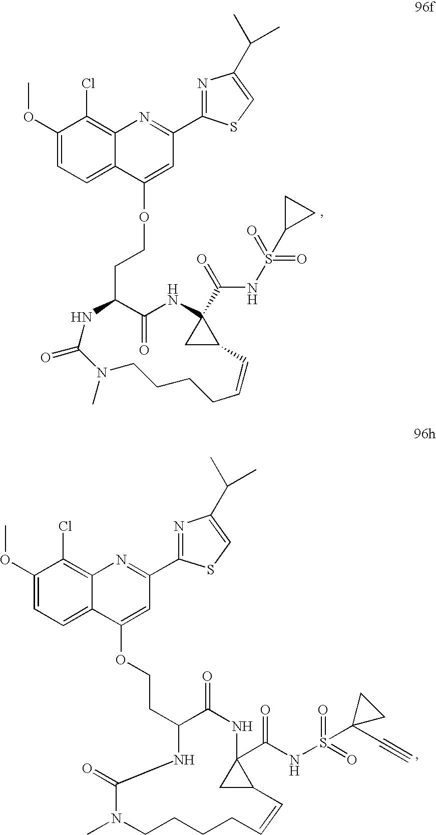

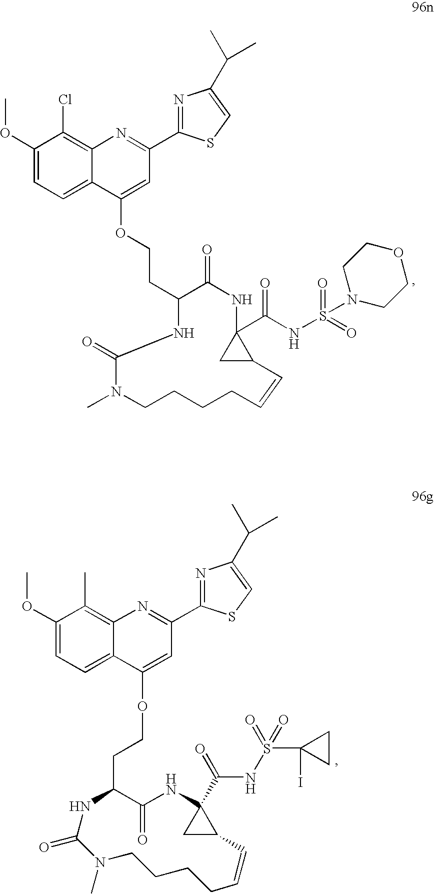

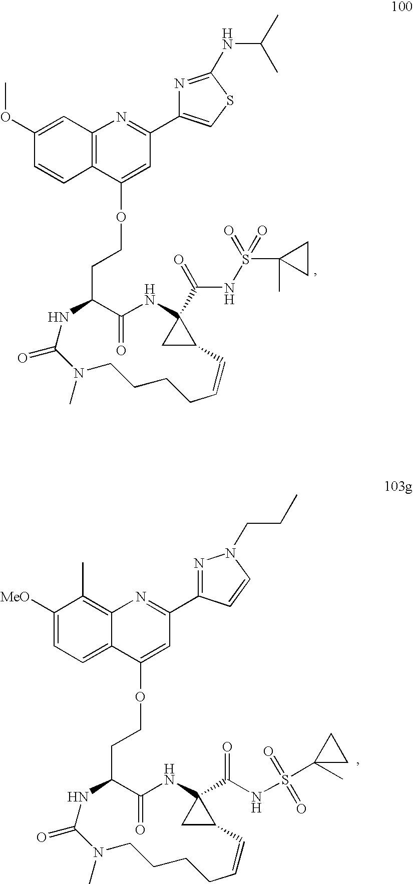

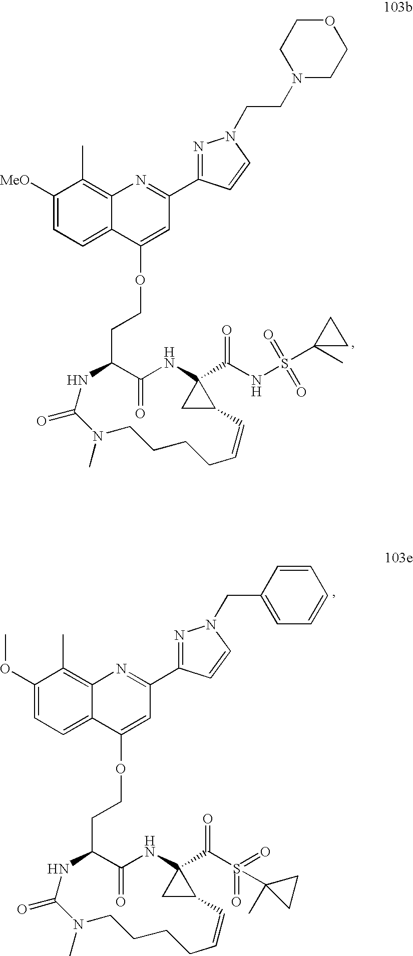

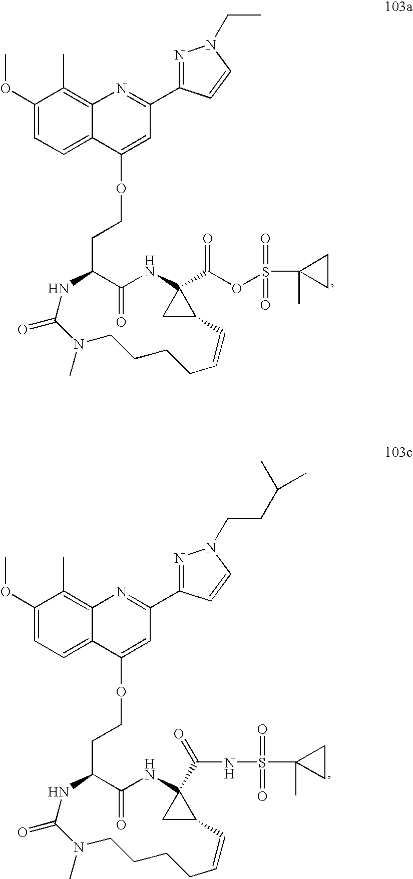

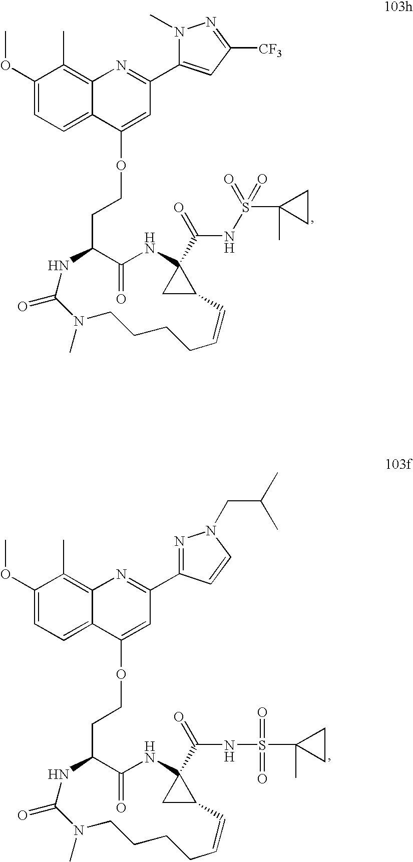

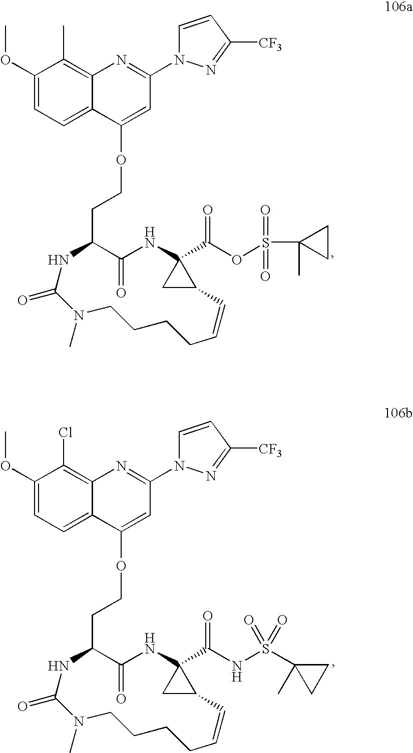

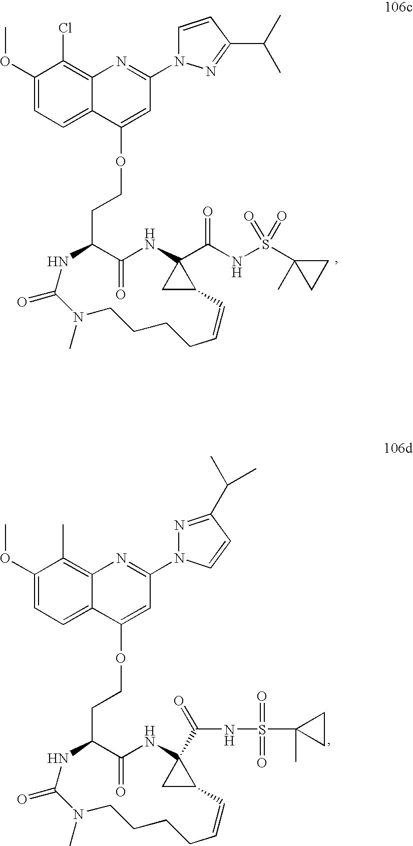

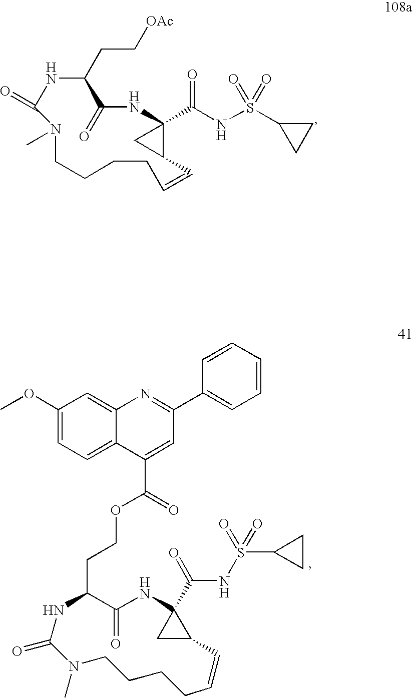

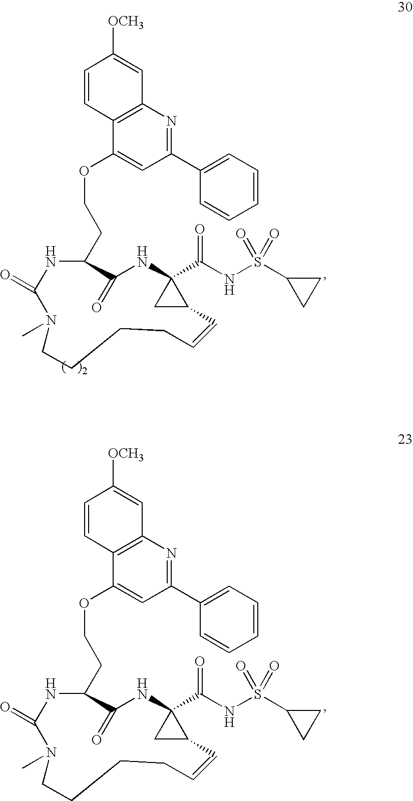

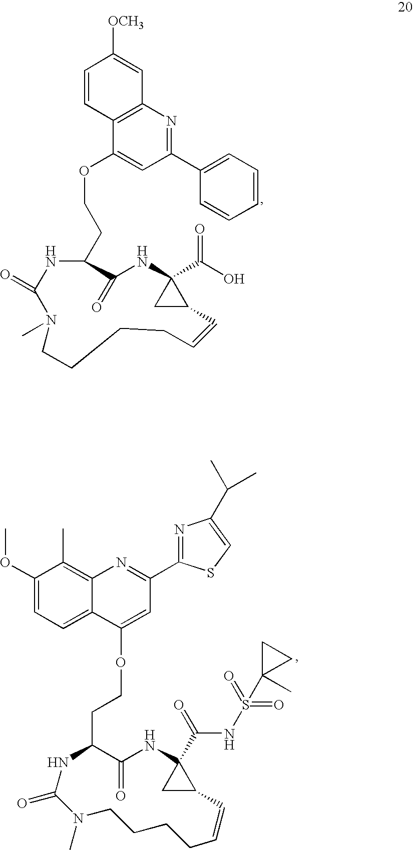

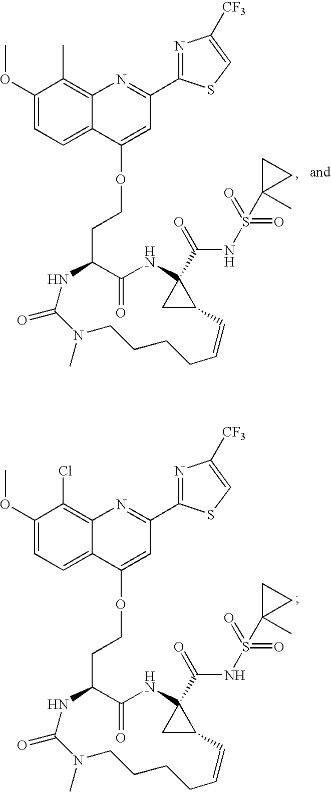

and pharmaceutically acceptable salts, solvates, and prodrugs thereof.

The compounds provided herein are intended to encompass all possible stereoisomers, unless a particular stereochemistry is specified. Where the compound provided herein contains an alkenyl or alkenylene group, the compound may exist as one or mixture of geometric cis/trans (or Z/E) isomers. Where structural isomers are interconvertible via a low energy barrier, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound provided herein may also be provided as a prodrug, which is a functional derivative of a compound provided herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

### Methods of Synthesis

The compounds provided herein can be prepared, isolated, or obtained by any method known to one of skill in the art. For an example, a compound of Formula I can be prepared as shown in Scheme 1.

Protected amino acid 1 is converted into compound 2 via Mitsunobu reaction. After the removal of the amino protecting group, compound 2 reacts with an unsaturated amine in the presence of CDI to form urea 3. After removal of the carboxyl protecting group, compound 3 is coupled with cyclopropylamine to afford compound 4. Subsequently, compound 4 is cyclized in the presence of metathesis catalyst to afford compound 5. The removal of the ethyl protecting group from the carboxyl group of compound 5 yields a macrocyclic acid 6, which is coupled with a variety of amines to form desired macrocyclic serine protease inhibitors, such as sulfonamide 7.

Alternatively, compound 4 is deprotected first and followed by coupling with an amine to produce amide 8, which is then cyclized to form a macrocyclic molecule 7.

The starting materials used in the synthesis of compounds provided herein are either commercially available or can be readily prepared. For example, protected homoserine, tert-butyl (S)-1-((benzyloxy)carbonyl)-3-hydroxypropylcarbamate, is prepared as described in J. Org. Chem. 1986, 51, 5047-5050.

### Pharmaceutical Composition

Provided herein are pharmaceutical compositions comprising a compound provided herein as an active ingredient, e.g., a compound of Formula I, including a single enantiomer, a mixture of enantiomers, an individual diastereomer, or a mixture of diastereomers thereof; or a pharmaceutically acceptable salt, solvate, or prodrug thereof, in combination with one or more pharmaceutically acceptable excipients or carriers. In certain embodiments, the pharmaceutical composition comprises at least one release controlling excipient or carrier. In certain embodiments, the pharmaceutical composition comprises at least one nonrelease controlling excipient or carrier. In certain embodiments, the pharmaceutical composition comprises at least one release controlling and at least one nonrelease controlling excipients or carriers.

The pharmaceutical compositions may be formulated in various dosage forms, including, but limited to, the dosage forms for oral, parenteral, or topical administration. The pharmaceutical compositions may also be formulated as modified release dosage forms, including, but not limited to, delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2003; Vol. 126).

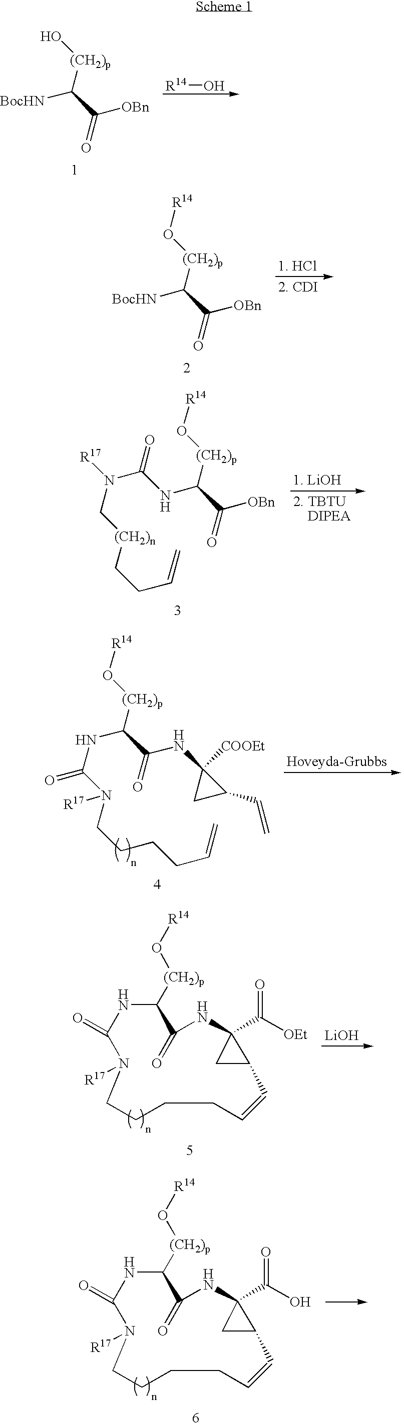

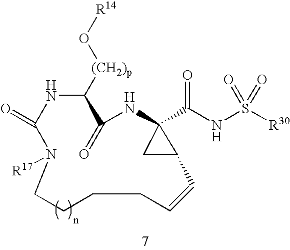

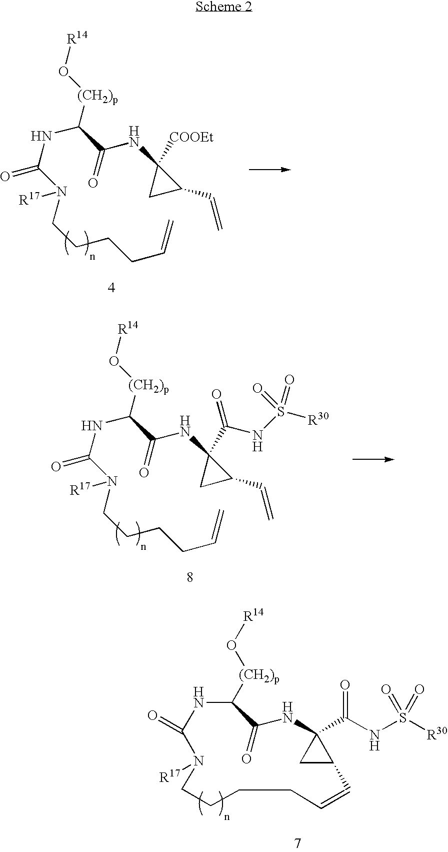

In one embodiment, the pharmaceutical compositions are provided in a dosage form for oral administration, which comprise a compound provided herein, e.g., a compound of Formula I, including a single enantiomer, a mixture of enantiomers, or a mixture of diastereomers thereof, or a pharmaceutically acceptable salt, solvate, or prodrug thereof; and one or more pharmaceutically acceptable excipients or carriers.

In another embodiment, the pharmaceutical compositions are provided in a dosage form for parenteral administration, which comprise a compound provided herein, e.g., a compound of Formula I, including a single enantiomer, a mixture of enantiomers, or a mixture of diastereomers thereof, or a pharmaceutically acceptable salt, solvate, or prodrug thereof; and one or more pharmaceutically acceptable excipients or carriers.

In yet another embodiment, the pharmaceutical compositions are provided in a dosage form for topical administration, which comprise a compound provided herein, e.g., a compound of Formula I, including a single enantiomer, a mixture of enantiomers, or a mixture of diastereomers thereof, or a pharmaceutically acceptable salt, solvate, or prodrug thereof; and one or more pharmaceutically acceptable excipients or carriers.

The pharmaceutical compositions provided herein may be provided in a unit- or multiple-dosage form. A unit-dosage form, as used herein, refers to a physically discrete unit suitable for administration to a subject, and packaged individually as is known in the art. Each unit-dose contains a predetermined quantity of the active ingredient(s) sufficient to produce the desired therapeutic effect, in association with the required pharmaceutically acceptable vehicle, carrier, diluent, excipient, or a mixture thereof. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include a vial, bottle of tablets or capsules, or bottle of pints or gallons.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the pharmaceutical compositions provided herein.

### A. Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. In certain embodiments, the binder or filler is present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. In certain embodiments, the pharmaceutical compositions provided herein contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. In certain embodiments, the pharmaceutical compositions provided herein contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Coloring agents include, but are not limited to, any of the approved, certified, water soluble FD&C dyes, water insoluble FD&C dyes suspended on alumina hydrate, and color lakes, and mixtures thereof. A color lake is the combination by adsorption of a water-soluble dye to a hydrous oxide of a heavy metal, resulting in an insoluble form of the dye. Flavoring agents include, but are not limited to, natural flavors extracted from plants, such as fruits, and synthetic blends of compounds which produce a pleasant taste sensation, such as peppermint and methyl salicylate. Sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Preservatives include, but are not limited to, glycerin, methyl and propylparaben, benzoic add, sodium benzoate and alcohol. Wetting agents include, but are not limited to, propylene glycol monostearate, sorbitan monooleate, diethylene glycol monolaurate, and polyoxyethylene lauryl ether. Solvents include, but are not limited to, glycerin, sorbitol, ethyl alcohol, and syrup. Examples of non-aqueous liquids utilized in emulsions include mineral oil and cottonseed oil. Organic acids include, but are not limited to, citric and tartaric acid. Sources of carbon dioxide include, but are not limited to, sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, enteric coated tablets, sugar-coated tablets, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered, press-coated, and dry-coated tablets.

The tablet dosage forms may be prepared from the active ingredient in powdered, crystalline, or granular forms, alone or in combination with a pharmaceutically acceptable vehicle, carrier, diluent, or excipient, or a mixture thereof; including a binder, disintegrant, controlled-release polymer, lubricant, diluent, and/or colorant. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as a dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including, but not limited to, methyl- and propyl-parabens, and sorbic acid. The liquid, semisolid, and solid dosage forms provided herein may be encapsulated in a capsule. Suitable liquid and semisolid dosage forms include, but are not limited to, solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including, but not limited to, emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action.

### B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, and dextrose and lactated Ringer's injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampoule, a vial, or a syringe. In certain embodiments, the multiple dosage parenteral formulations contain an antimicrobial agent at bacteriostatic or fungistatic concentrations. In certain embodiments, the parenteral formulations provided herein are sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein may be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions may also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including lard, benzoinated lard, olive oil, cottonseed oil, and other oils; white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; and emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream bases can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout a liquid carrier. Suitable gelling agents include crosslinked acrylic acid polymers, such as carbomers, carboxypolyalkylenes, CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; and glycerinated gelatin. Combinations of the various vehicles may be used. Rectal and vaginal suppositories may further comprise antioxidants as described herein, including bisulfite and sodium metabisulfite. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; or nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator may be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of monohydrates. Other suitable excipients or carriers include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

### D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz Ed., Wiley, 1999).

In one embodiment, the pharmaceutical compositions provided herein is formulated in a modified release dosage form using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In certain embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethylene terephthalate, natural rubber, silicone rubbers, polydimethylsiloxanes, silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form may be prepared by methods known to those skilled in the art, including direct compression, dry or wet granulation followed by compression, or melt-granulation followed by compression.

### 2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents includes osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters, poly-(methacrylic) acids and esters, and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings as described in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as an AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, or dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, those described in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

Provided herein are methods for treating or preventing a hepatitis C viral infection in a subject, which comprises administering to a subject a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof. In one embodiment, the subject is a mammal. In another embodiment, the subject is a human.

Additionally, provided herein is a method for inhibiting replication of a virus in a host, which comprises contacting the host with a therapeutically effective amount of the compound of Formula I, including a single enantiomer, a mixture of an enantiomeric pair, an individual diastereomer, or a mixture of diastereomers thereof; or a pharmaceutically acceptable salt, solvate, or prodrug thereof. In one embodiment, the host is a cell. In another embodiment, the host is a human cell. In yet another embodiment, the host is a mammal. In still another embodiment, the host is human.

In certain embodiments, administration of a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 10%, 20%, 30%, 40%, 50%, 60%, 70%, 80%, 90%, 95%, 99% or more reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer.

In certain embodiments, administration of a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 1, 2, 3, 4, 5, 10, 15, 20, 25, 50, 75, 100-fold or more reduction in the replication of the virus relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 10%, 20%, 30%, 40%, 50%, 60%, 70%, 80%, 90%, 95%, 99% or more reduction in the viral titer relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 1, 2, 3, 4, 5, 10, 15, 20, 25, 50, 75, 100 or more fold reduction in the viral titer relative to a subject without administration of the compound, as determined at 1 day, 2 days, 3 days, 4 days, 5 days, 10 days, 15 days, or 30 days after the administration by a method known in the art.

Further provided herein is a method for inhibiting the replication of an HCV virus, which comprises contacting the virus with a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof.

In certain embodiments, the contacting of the virus with a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 10%, 20%, 30%, 40%, 50%, 60%, 70%, 80%, 90%, 95%, 99% or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 1, 2, 3, 4, 5, 10, 15, 20, 25, 50, 75, 100-fold or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 10%, 20%, 30%, 40%, 50%, 60%, 70%, 80%, 90%, 95%, 99% or more reduction in the viral titer relative to the virus without such contact, as determined at 1 day, 2 days, 3 days, 4 days, 5 days, 10 days, 15 days, or 30 days after the initial contact by a method known in the art.

In certain embodiments, the contacting of the virus with a therapeutically effective amount of a compound provided herein, including a single enantiomer, a mixture of an enantiomeric pair, an individual diastereomer, or a mixture of diastereomers thereof; or a pharmaceutically acceptable salt, solvate, or prodrug thereof, results in a 1, 2, 3, 4, 5, 10, 15, 20, 25, 50, 75, 100 or more fold reduction in the viral titer relative to the virus without such contact, as determined at 1 day, 2 days, 3 days, 4 days, 5 days, 10 days, 15 days, or 30 days after the initial contact, by a method known in the art.

Also provided herein is a method for treating, preventing, or ameliorating one or more symptoms of a liver disease or disorder associated with an HCV infection, comprising administering to a subject a therapeutically effective amount of the compound provided herein, including a single enantiomer, a mixture of an enantiomeric pair, an individual diastereomer, or a mixture of diastereomers thereof; or a pharmaceutically acceptable salt, solvate, or prodrug thereof. Non-limiting examples of diseases associated with HCV infection include chronic hepatitis, cirrhosis, hepatocarcinoma, or extra hepatic manifestation.

Provided herein is a method for inhibiting the activity of a serine protease, which comprises contacting the serine protease with an effective amount of a compound provided herein, including a single enantiomer, a mixture of an enantiomeric pair, an individual diastereomer, or a mixture of diastereomers thereof; or a pharmaceutically acceptable salt, solvate, or prodrug thereof. In one embodiment, the serine protease is hepatitis C NS3 protease.

Depending on the condition, disorder, or disease, to be treated and the subject's condition, a compound provided herein may be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1000 milligram, from about 0.1 to about 500 milligrams, or from 0.5 about to about 100 milligram active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds provided herein may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to treat, prevent, or manage a disease or disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The compound provided herein can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameters of the drug can be altered by such combination or alternation therapy. In general, combination therapy is typically preferred over alternation therapy because it induces multiple simultaneous stresses on the virus.

In certain embodiments, the compound provided herein is combined with one or more agents selected from the group consisting of an interferon, ribavirin, amantadine, an interleukin, a NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, and a ribozyme.

In certain embodiments, the compound provided herein is combined with a HCV protease inhibitor, including, but not limited to, Medivir HCV protease inhibitor (Medivir/Tobotec); ITMN-191 (InterMune), SCH 503034 (Schering), VX950 (Vertex); substrate-based NS3 protease inhibitors as disclosed in WO 98/22496; Attwood et al., Antiviral Chemistry and Chemotherapy 1999, 10, 259-273; DE 19914474; WO 98/17679; WO 99/07734; non-substrate-based NS3 protease inhibitors, such as 2,4,6-trihydroxy-3-nitro-benzamide derivatives (Sudo et al., Biochem. Biophys. Res. Commun. 1997, 238, 643-647), RD3-4082, RD3-4078, SCH 68631, and a phenanthrenequinone (Chu et al., Tetrahedron Letters 1996, 37, 7229-7232); SCH 351633 (Chu et al., Bioorganic and Medicinal Chemistry Letters 1999, 9, 1949-1952); Eglin c, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinàs-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; U.S. Pat. App. Pub. Nos.: 2002/0016294, 2002/0016442; 2002/0037998; 2002/0032175; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781; WO 98/17679; WO 98/22496; WO 99/07734; WO 00/059929; WO 00/09543; WO 02/060926; WO 02/08187; WO 02/008251; WO 02/008256; WO 02/08198; WO 02/48116; WO 02/48157; WO 02/48172; WO 03/053349; WO 03/064416; WO 03/064456; WO 03/099274; WO 03/099316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; and WO 2007/056120.

Other protease inhibitors include thiazolidine derivatives, such as RD-1-6250, RD4 6205, and RD4 6193, which show relevant inhibition in a reverse-phase HPLC assay with an NS3/4A fusion protein and NS5A/5B substrate (Sudo et al., Antiviral Research 1996, 32, 9-18); thiazolidines and benzanilides identified in Kakiuchi et al., FEBS Lett. 1998, 421, 217-220; Takeshita et al., Analytical Biochemistry 1997, 247, 242-246.

Suitable helicase inhibitors include, but are not limited to, those disclosed in U.S. Pat. No. 5,633,358; and WO 97/36554.

Suitable nucleotide polymerase inhibitors include, but are not limited to, gliotoxin (Ferrari et al., Journal of Virology 1999, 73, 1649-1654), and the natural product cerulenin (Lohmann et al., Virology 1998, 249, 108-118).

Suitable interfering RNA (iRNA) based antivirals include, but are not limited to, short interfering RNA (siRNA) based antivirals, such as Sima-034 and those described in WO/03/070750, WO 2005/012525, and U.S. Pat. Pub. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; Galderisi et al., Journal of Cellular Physiology 1999, 181, 251-257);

Suitable inhibitors of IRES-dependent translation include, but are not limited to, those described in Japanese Pat. Pub. Nos.: JP 08268890 and JP 10101591.

Suitable ribozymes include those disclosed in, for example, U.S. Pat. Nos. 6,043,077; 5,869,253 and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. Pub. Nos. 2004/0121980; 2005/0009737; 2005/0038240; and 2006/0040890; WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, INTRON® A (interferon alfa-2b) and PEGASYS® (Peginterferon alfa-2a); ROFERON® A (recombinant interferon alfa-2a), INFERGEN® (interferon alfacon-1), and PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, one or more compounds provided herein are administered in combination or alternation with an anti-hepatitis C virus polymerase inhibitor, such as ribavirin, viramidine, NM 283 (valopicitabine), PSI-6130, R1626, HCV-796, or R7128.

In certain embodiments, the one or more compounds provided herein are administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), and PEG-INTRON® (pegylated interferon alfa-2b),

In certain embodiments, one or more compounds provided herein are administered in combination or alternation with an anti-hepatitis C virus protease inhibitor, such as ITMN-191, SCH 503034, VX950 (telaprevir), or Medivir HCV protease inhibitor.

In certain embodiments, one or more compounds provided herein are administered in combination or alternation with an anti-hepatitis C virus vaccine, including, but not limited to, TG4040, PEVIPRO™, CGI-5005, HCV/MF59, GV1001, IC41, and INNO0101 (E1).

In certain embodiments, one or more compounds provided herein are administered in combination or alternation with an anti-hepatitis C virus monoclonal antibody, such as AB68 or XTL-6865 (formerly HepX-C); or an anti-hepatitis C virus polyclonal antibody, such as cicavir.

In certain embodiments, one or more compounds provided herein are administered in combination or alternation with an anti-hepatitis C virus immunomodulator, such as ZADAXIN® (thymalfasin), NOV-205, or oglufanide.

In certain embodiments, one or more compounds provided herein are administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide) or PYN17.

In certain embodiments, the compounds provided herein can be combined with one or more steroidal drugs known in the art, including, but not limited to the group including, aldosterone, beclometasone, betamethasone, deoxycorticosterone acetate, fludrocortisone, hydrocortisone (cortisol), prednisolone, prednisone, methylprednisolone, dexamethasone, and triamcinolone.

In certain embodiments, the compounds provided herein can be combined with one or more antibacterial agents known in the art, including, but not limited to the group including amikacin, amoxicillin, ampicillin, arsphenamine, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, cloxacillin, colistin, dalfopristin, demeclocycline, dicloxacillin, dirithromycin, doxycycline, erythromycin, enrofloxacin, ertepenem, ethambutol, flucloxacillin, fosfomycin, furazolidone, gatifloxacin, geldanamycin, gentamicin, herbimycin, imipenem, isoniazid, kanamycin, levofloxacin, linezolid, lomefloxacin, loracarbef, mafenide, moxifloxacin, meropenem, metronidazole, mezlocillin, minocycline, mupirocin, nafcillin, neomycin, netilmicin, nitrofurantoin, norfloxacin, ofloxacin, oxytetracycline, penicillin, piperacillin, platensimycin, polymyxin B, prontocil, pyrazinamide, quinupristine, rifampin, roxithromycin, spectinomycin, streptomycin, sulfacetamide, sulfamethizole, sulfamethoxazole, teicoplanin, telithromycin, tetracycline, ticarcillin, tobramycin, trimethoprim, troleandomycin, trovafloxacin, and vancomycin.

In certain embodiments, the compounds provided herein can be combined with one or more antifungal agents known in the art, including, but not limited to the group including amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

In certain embodiments, the compounds provided herein can be combined with one or more anticoagulants known in the art, including, but not limited to the group including acenocoumarol, argatroban, bivalirudin, lepirudin, fondaparinux, heparin, phenindione, warfarin, and ximelagatran.

In certain embodiments, the compounds provided herein can be combined with one or more thrombolytics known in the art, including, but not limited to the group including anistreplase, reteplase, t-PA (alteplase activase), streptokinase, tenecteplase, and urokinase.

In certain embodiments, the compounds provided herein can be combined with one or more non-steroidal anti-inflammatory agents known in the art, including, but not limited to, aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin.

In certain embodiments, the compounds provided herein can be combined with one or more antiplatelet agents known in the art, including, but not limited to, abciximab, cilostazol, clopidogrel, dipyridamole, ticlopidine, and tirofibin.

The compounds provided herein can also be administered in combination with other classes of compounds, including, but not limited to, endothelin converting enzyme (ECE) inhibitors, such as phosphoramidon; thromboxane receptor antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abciximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopeptidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; beta-adrenergic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, and vardenafil); protein tyrosine kinase inhibitors; antiinflammatories; antiproliferatives, such as methotrexate, FK506 (tacrolimus), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone antagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathioprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

In certain embodiments, the pharmaceutical compositions provided herein further comprise a second antiviral agent as described herein. In one embodiment, the second antiviral is selected from the group consisting of an interferon, ribavirin, an interleukin, an NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, and a ribozyme. In another embodiment, the second antiviral agent is an interferon. In yet another embodiment, the t interferon is selected from the group consisting of pegylated interferon alpha 2a, interferon alphcon-1, natural interferon, ALBUFERON®, interferon beta-1a, omega interferon, interferon alpha, interferon gamma, interferon tau, interferon delta, and interferon gamma-1b.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein, including a single enantiomer, a mixture of an enantiomeric pair, an individual diastereomer, or a mixture of diastereomers thereof; or a pharmaceutically acceptable salt, solvate, or prodrug thereof.

In certain embodiments, the kit includes a container comprising a dosage form of the compound provided herein, including a single enantiomer, a mixture of an enantiomeric pair, an individual diastereomer, or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: RT (room temperature); g (grams); mg (milligrams); mL (milliliters); μL (microliters); mM (millimolar); μM (micromolar); Hz (Hertz); MHz (megahertz); mmol (millimoles); hr (hours); min (minutes); TLC (thin layer chromatography); HPLC (high performance liquid chromatography); SCX (strong cation exchange); MS (mass spectrometry); ESI (electrospray ionization); Rₜ (retention time); SiO₂ (silica); CD₃OD (deuterated methanol); CDCl₃ (deuterated chloroform); DMSO-d₆ (deuterated dimethylsulfoxide); CHCl₃ (chloroform); DCE (1,2-dichloroethane); DCM (dichloromethane); DMF (N,N-dimethylormamide); DMSO (dimethylsulfoxide); EtOH (ethanol); Et₂O (diethyl ether); EtOAc (ethyl acetate); MeOH (methanol); PE (petroleum ether); THF (tetrahydrofuran); HCl (hydrochloric acid); Cs₂CO₃ (cesium carbonate); LiOH (lithium hydroxide); KOH (potassium hydroxide); NaOH (sodium hydroxide); DBU (1,8-diazabicyclo[5.4.0]undec-7-ene; DIPEA (N,N-diisopropylethylamine); TEA (triethylamine); CDI (carbonyldiimidazole); DIAD (Diisopropyl azodicarboxylate); NFSI (N-fluorobenzenesulfonimide); PPA (polyphosphoric acid); TBAF (tetra-n-butylammonium fluoride); TBTU (O-(benzotriazol-1-yl)-N,N,N′,N′-tetramethyluronium tetrafluoroborate); Ac (acetyl); Bn (benzyl); Boc (tert-butoxylcarbony); Et (ethyl); iPr (isopropyl); Me (methyl); tBu (tert-butyl); and Ts (tosylate).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

### Example 1

### Preparation of N-methyl-ω-alkenyl-1-amine tosylate salts 12

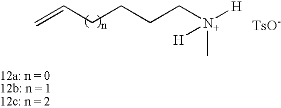

The syntheses of N-methyl-ω-alkenyl-1-amine tosylate salts 12 are shown in Scheme 3.

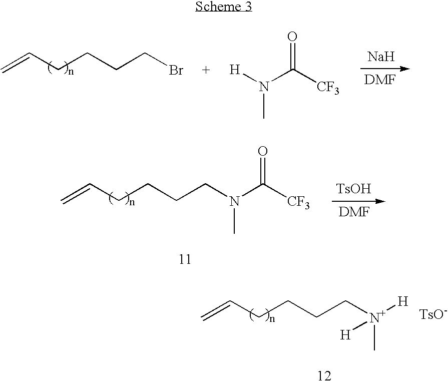

Step 1: Preparation of 2,2,2-trifluoro-N-(hex-5-enyl)-N-methylacetamide 11a. Sodium hydride (60% dispersion in mineral oil, 31.5 g, 1.28 eq.) was slowly added under nitrogen atmosphere to a solution of N-methyl-2,2,2-trifluoroacetamide (100 g, 1.28 eq.) in DMF (500 mL) at 0° C. The reaction mixture was stirred for 90 min at 0° C., and then 6-bromo-1-hexene (100 g, 1 eq.) was added dropwise over 45 min. The reaction mixture was allowed to warm up to room temperature, and stirred for 3 days at room temperature. The reaction mixture was then poured into water and extracted tree time with EtOAc. The combined organics layers were dried over anhydrous sodium sulphate and concentrated under reduced pressure. The residue was purified by flash chromatography on silica gel to produce compound 11a as colorless oil in 56% yield.

¹H NMR (DMSO-d₆, 400 MHz) δ 1.27-1.38 (m, 2H), 1.48-1.60 (m, 2H), 2.00-2.06 (m, 2H), 2.93-3.07 (2m, 3H), 3.35-3.40 (m, 2H), 4.92-5.04 (m, 2H), 5.73-5.83 (m, 1H).

Step 2: N-Methylhex-5-en-1-amine tosylate salt 12a. At room temperature, compound 11a (71.88 g, 1 eq.) and p-toluene sulfonic acid (74.4 g, 1.2 eq.) were dissolved in MeOH (640 mL). The reaction mixture was refluxed for 7 days. The solvent was then removed under vacuum, and the residue was recrystallized in acetone. The product was isolated by filtration, dried over P₂O₅ to give compound 12a as a white powder in 76% yield.

¹H NMR (CDCl₃, 400 MHz): δ 1.38 (q, J=7.76 Hz, 2H), 1.71 (q, J=7.76 Hz, 2H), 1.99 (q, J=6.98 Hz, 2H), 2.38 (s, 3H), 2.70 (t, J=5.17 Hz, 3H), 2.87-2.93 (m, 2H), 4.92-4.99 (m, 2H), 5.67-5.73 (m, 1H), 7.20 (d, J=7.76 Hz, 2H), 7.75 (d, J=7.76 Hz, 2H), 8.62 (br s, 2H).

Step 3: N-Methylhept-5-en-1-amine tosylate salt 12b. Compound 12b was synthesized from 7-bromo-heptene as a white solid in quantitative yield, following the procedure as described for compound 12a.

¹H NMR (CDCl₃, 400 MHz) δ 1.38 (q, J=7.76 Hz, 2H), 1.71 (q, J=7.76 Hz, 2H), 1.80 (q, J=6.98 Hz, 2H), 1.99 (q, J=6.98 Hz, 2H), 2.38 (s, 3H), 2.70 (t, J=5.17 Hz, 3H), 2.87-2.93 (m, 2H), 4.92-4.99 (m, 2H), 5.67-5.73 (m, 1H), 7.20 (d, J=7.76 Hz, 2H), 7.75 (d, J=7.76 Hz, 2H), 8.62 (br s, 2H).

Step 4: N-Methyloct-5-en-1-amine tosylate salt 12c. Compound 12c was synthesized from 7-bromo-octene as a white powder in quantitative yield, following the procedure as described for compound 12a.

¹H NMR (CDCl₃, 400 MHz) δ 1.38 (q, J=7.76 Hz, 2H), 1.71 (q, J=7.76 Hz, 2H), 1.80 (q, J=6.98 Hz, 2H), 1.90 (q, J=6.9 Hz, 2H), 1.99 (q, J=6.98 Hz, 2H), 2.38 (s, 3H), 2.70 (t, J=5.17 Hz, 3H), 2.87-2.93 (m, 2H), 4.92-4.99 (m, 2H), 5.67-5.73 (m, 1H), 7.20 (d, J=7.76 Hz, 2H), 7.75 (d, J=7.76 Hz, 2H), 8.62 (brs, 2H).

### Example 2

### Preparation of Macrocyclic Compound 20

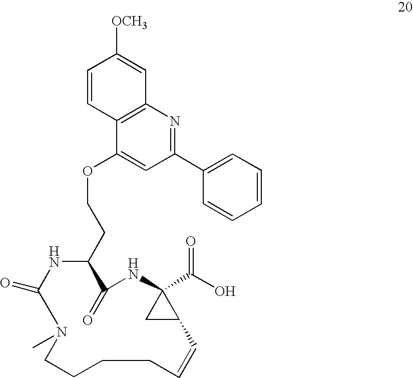

The synthesis of macrocyclic compound 20 is shown in Scheme 4.

Step 1: To a solution of tert-butyl (S)-1-((benzyloxy)carbonyl)-3-hydroxypropylcarbamate (1.85 g, 6.0 mmol), 7-methoxy-2-phenylquinolin-4-ol (1.55 g, 6.2 mmol), and triphenylphosphine (3.14 g, 12.0 mmol) in THF (60 mL) was added dropwise DIAD (2.36 mL, 12.0 mmol) under nitrogen at 0° C. (ice bath). The solvents were evaporated and the crude residue was dissolved in EtOAc. The organic layer was washed with a NaHCO₃ saturated solution, followed by brine, then dried over anhydrous Na₂SO₄, and evaporated in vacuo. The crude product was purified by chromatography on silica gel with PE/EtOAc (9:1 to 1:1, v/v) to afford compound 13 as a thick oil contaminated with reduced DIAD (5.33 g), which was used without further purification in the next step. A pure analytical sample was obtained by trituration in isopropylacetate to give benzylester 13 as a white solid.

¹H NMR (DMSO-d₆, 400 MHz) δ 1.33 (s, 9H), 2.24-2.37 (m, 2H), 3.92 (s, 3H), 4.34-4.46 (m, 3H), 5.14 (d, J=12.6 Hz, 1H), 5.09 (d, J=12.6 Hz, 1H), 7.15 (dd, J=9.2 and 2.4 Hz, 1H), 7.27-7.30 (m, 5H), 7.36-7.38 (m, 2H), 7.46-7.55 (m, 4H), 8.00 (d, J=9.2 Hz, 1H), 8.23-8.25 (m, 2H); MS (ESI, El⁺) m/z=543 (MH⁺).

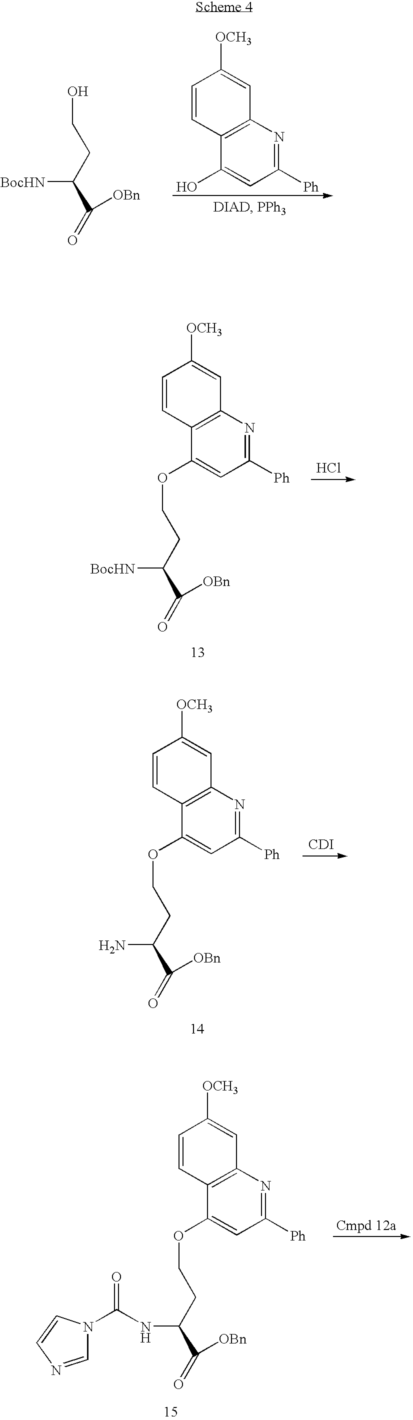

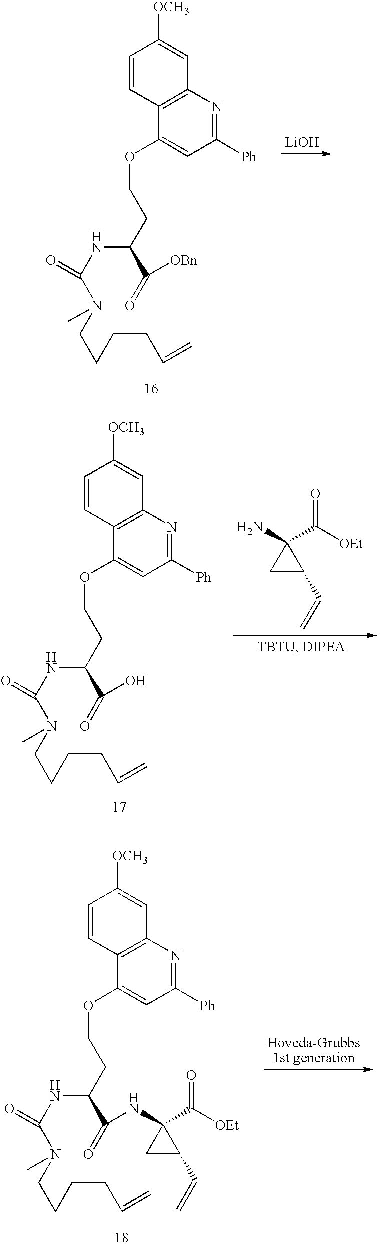

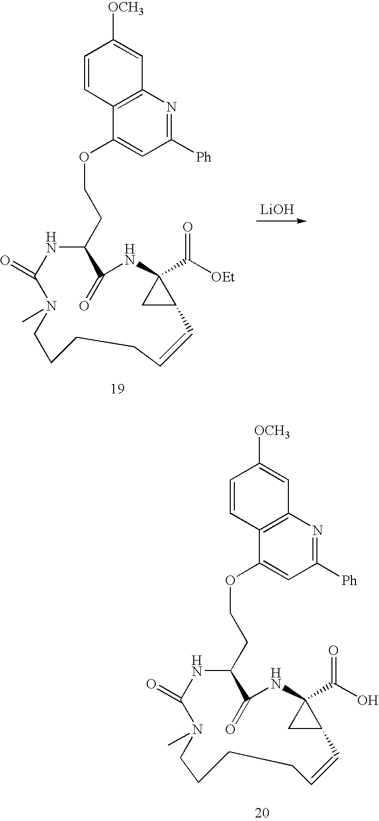

Step 2: To a solution of benzylester 13 (contaminated with reduced DIAD, 4.805 g, 8.85 mmol) in anhydrous Et₂O was added a solution of 4M HCl in dioxane (13.2 mL, 53.1 mmol) under N₂. The solution was stirred at RT for 48 hr, and then the precipitated solid was filtrated and washed with anhydrous Et₂O, dried under vacuum to afford compound 14 as a white solid (1.976 g).

¹H NMR (DMSO-d₆, 400.13 MHz) δ 2.56-2.61 (m, 2H), 3.95 (s, 3H), 4.41-4.42 (m, 1H), 4.81-4.83 (m, 2H), 5.17-5.18 (m, 2H), 7.22-7.29 (m, 5H), 7.37 (dd, J=9.16 and 1.96 Hz, 1H) 7.59-7.69 (m, 4H), 8.15-8.27 (m, 4H), 9.05 (br, 3H); MS (ESI, El⁺) m/z=443 (MH⁺).

Step 3: To a solution of CDI (1.22 g, 7.52 mmol) and TEA (1 mL, 7.52 mmol) in anhydrous DCM (70 mL) was added compound 14. The reaction mixture was stirred at RT overnight. The solution was diluted with DCM and then washed twice with H₂O, dried over anhydrous Na₂SO₄, and concentrated in vacuo to afford imidazole 15 as an off-white solid (3.084 g).

¹H NMR (DMSO-d₆, 400.13 MHz) δ 2.40-2.60 (m, 2H), 3.91 (s, 3H), 4.40-4.60 (m, 2H), 4.70-4.80 (m, 1H), 5.18 (s, 2H), 7.15 (dd, J=8.6 and 2.4 Hz, 1H), 7.20-7.30 (m, 6H), 7.45-7.55 (m, 4H), 7.70 (s, 1H), 7.95 (d, J=7.6 Hz, 1H), 8.30 (d, J=7.92, 2H), 8.40 (s, 1H), 9.00 (d, J=8.6 Hz, 1H); MS (ESI, El⁺) m/z=537 (MH⁺).

Step 4: A solution of imidazole 15 (2.046 g, 3.82 mmol), N-methylhex-5-en-1-amine tosylated (Ts) salt 12a (1.30 g, 4.58 mmol), and TEA (0.650 mL, 4.58 mmol) was heated at reflux for 3 hr under N₂. After cooling to RT, the solvents were evaporated in vacuo and the crude residue was purified by column chromatography on silica gel with PE/EtOAc (7:3 to 1:1, v/v) to afford alkene 16 as a white solid (1.73 g).

¹H NMR (DMSO-d₆, 400.13 MHz) δ 1.22-1.24 (m, 2H), 1.34-1.36 (m, 2H), 1.90-1.92 (m, 2H), 2.20-2.40 (m, 2H), 2.20-2.40 (s, 3H), 3.14-3.17 (m, 2H), 3.91 (s, 3H), 4.40-4.50 (m, 3H), 4.84 (d, J=10.1 Hz, 1H), 4.90 (d, J=17.0 Hz, 1H), 5.10 (s, 2H), 5.64-5.69 (m, 1H), 6.65 (d, J=7.92 Hz, 1H), 7.14 (dd, J=9.08 and 2.52 Hz, 1H), 7.29-7.30 (m, 5H), 7.37-7.38 (m, 2H), 7.50-7.52 (m, 3H), 8.00 (d, J=9.08 Hz, 1H), 8.23 (d, J=7.92 Hz, 2H); MS (ESI, El⁺) m/z=582 (MH⁺).

Step 5: A solution of alkene 16 (1.720 g, 2.95 mmol) and LiOH (0.213 g, 8.86 mmol) in a mixture of water (80 mL) and THF (80 mL) was stirred overnight at room temperature. The volatile solvent was then evaporated and the aqueous layer was acidified with a solution of 1M HCl to pH 3 to 4. The aqueous layer was extracted three times with EtOAc. The combined organic layers were dried over anhydrous Na₂SO₄ and concentrated in vacuo to afford the acid 17 as a white solid (1.561 g), which was used in the next step without further purification.

¹H NMR (DMSO-d₆, 400.13 MHz) δ 1.16-1.41 (m, 4H), 1.90-1.98 (m, 2H), 2.23-2.42 (m, 2H), 2.76 (s, 3H), 3.08-3.20 (m, 2H), 2.96 (s, 3H), 4.36-4.45 (m, 1H), 4.46-4.60 (m, 2H), 4.83-4.94 (m, 2H), 5.64-5.75 (m, 1H), 6.45 (d, J=2.4 Hz, 1H), 7.28 (br, 1H), 7.40-7.50 (m, 2H), 7.52-7.64 (m, 3H), 8.05-8.15 (m, 1H), 8.18-8.25 (m, 2H), 12.60 (br, 1H); MS (ESI, El⁺) m/z=492 (MH⁺).

Step 6: To a solution of acid 17 (1.551 g, 2.95 mmol), (1R,2S)-ethyl 1-amino-2-vinylcyclopropanecarboxylate (Ts salt, 0.965 g, 2.95 mmol), DIPEA (1 mL, 5.90 mmol) in anhydrous DMF (50 mL) was added TBTU (0.947 g, 2.95 mmol) under nitrogen. The reaction mixture was stirred at RT overnight. The DMF was then evaporated under vacuum, water was added and the aqueous layer extracted three times with EtOAc. The combined organic layers were dried over anhydrous Na₂SO₄ and concentrated in vacuo. The crude residue was purified by column chromatography on silica gel with PE/EtOAc (1:1, v/v) to afford diene 18 (1.388 g) as thick slight yellow oil, which solidified on standing.

¹H NMR (DMSO-d₆, 400.13 MHz) δ 1.08-1.13 (m, 3H), 1.17-1.29 (m, 2H), 1.30-1.40 (m, 2H), 1.60-1.69 (m, 1H), 1.86-1.95 (m, 2H), 2.08-2.40 (m, 4H), 2.76 (s, 3H), 3.07-3.22 (m, 2H), 3.92 (s, 3H), 3.96-4.06 (m, 2H), 4.35-4.47 (m, 3H), 4.82-4.92 (m, 2H), 5.07-5.11 (m, 1H), 5.24-5.30 (m, 1H), 5.56-5.75 (m, 2H), 6.23 (dd, J=12.0 and 8.4 Hz, 1H), 7.16 (dd, J=9.2 and 2.4 Hz, 1H), 7.37 (br, 2H), 7.45-7.53 (m, 3H), 8.08-8.13 (m, 1H), 8.24 (d, J=2.8 Hz, 2H), 8.80 (s, 1H); MS (ESI, El⁺) m/z=629 (MH⁺).

Step 7: A mixture of diene 18 (81 mg, 0.13 mmol) and Hoveyda-Grubbs first generation (15 mg, 20% mol) in degassed DCM (80 mL) was stirred at reflux overnight under N₂. After cooling, the solvent was evaporated and the crude residue was purified by semi-preparative HPLC (C18) to give macrocyclic ester 19 as a white solid (17 mg).

¹H NMR (DMSO-d₆, 400.13 MHz) δ 1.09 (t, J=7.0 Hz, 3H), 1.13-1.17 (m, 2H), 1.35-1.54 (m, 4H), 1.66-1.72 (m, 3H), 2.81 (s, 3H), 3.16 (d, J=5.2 Hz, 2H), 3.93 (s, 3H), 3.98 (q, J=6.8 Hz, 2H), 4.12-4.27 (m, 3H), 4.43-4.50 (m, 2H), 5.40-5.54 (m, 2H), 6.10 (br, 1H), 7.17 (dd, J=9.2 and 2.4 Hz, 1H), 7.36-7.40 (m, 2H), 7.46-7.54 (m, 3H), 8.14 (d, J=9.2 Hz, 1H), 8.22-8.27 (m, 2H), 8.47 (s, 1H); MS (ESI, El⁺) m/z=601 (MH⁺).

Step 8: A solution of macrocyclic ester 19 (13 mg, 0.022 mmol) and LiOH (3 mg, 5 eq.) in a mixture of water (2 mL) and THF (2 mL) was stirred overnight at room temperature. The volatile solvent was then evaporated and the aqueous layer was acidified with a solution of 1M HCl to pH 3 to 4. The aqueous layer was extracted three times with EtOAc. The combined organic layers were washed with brine, dried over anhydrous Na₂SO₄, and concentrated in vacuo to afford macrocyclic acid 20 as a white solid (11 mg), which was used in the next step without further purification.

¹H NMR (DMSO-d₆, 400.13 MHz) δ 1.05-1.30 (m, 5H), 1.34-1.57 (m, 3H), 1.60-1.80 (m, 2H), 2.10-2.30 (m, 2H), 2.81 (s, 3H), 3.93 (s, 3H), 4.04-4.30 (m, 2H), 4.40-4.52 (m, 2H), 5.39-5.54 (m, 2H), 6.14 (br, 1H), 7.13-7.20 (m, 1H), 7.36-7.41 (m, 2H), 7.44-7.57 (m, 3H), 8.14 (d, J=8.4 Hz, 1H), 8.21-8.28 (m, 2H), 8.37 (br, 1H), 12.35 (br, 1H); MS (ESI, El⁺) m/z 573 (MH⁺).

### Example 3

### Preparation of Macrocyclic Compound 23

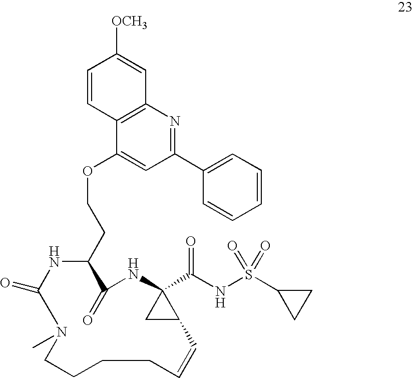

The synthesis of macrocyclic compound 23 is shown in Scheme 5.

Step 1: A solution of diene 18 (497 mg, 0.79 mmol) and LiOH (103 mg, 3.95 mmol) in a mixture of water (25 mL) and THF (25 mL) was stirred overnight at room temperature. The volatile solvent was then evaporated and the aqueous layer was acidified with a solution of 1M HCl to pH 3 to 4. The aqueous layer was extracted three times with DCM. The combined organic layers were dried over anhydrous Na₂SO₄ and concentrated in vacuo to afford acid 21 as a white solid (474 mg), which was used in the next step without further purification.

¹H NMR (DMSO-d₆, 400.13 MHz) δ 1.18-1.34 (m, 5H), 1.57-1.64 (m, 1H), 1.88-1.93 (m, 2H), 2.05-2.22 (m, 2H), 2.29-2.44 (m, 1H), 2.75 (s, 3H), 3.06-3.16 (m, 2H), 3.98 (s, 3H), 4.39-4.50 (m, 1H), 4.52-4.71 (m, 2H), 4.83-4.89 (m, 2H), 5.06-5.10 (m, 1H), 5.22-5.29 (m, 1H), 5.63-5.75 (m, 2H), 6.23-6.28 (m, 1H), 7.34-7.48 (m, 1H), 7.53-7.76 (m, 5H), 7.16 (d, J=7.6 Hz, 2H), 8.25-8.40 (m, 1H), 8.73 (br, 1H), 12.50 (br, 1H); MS (ESI, El⁺) m/z=601 (MH⁺).

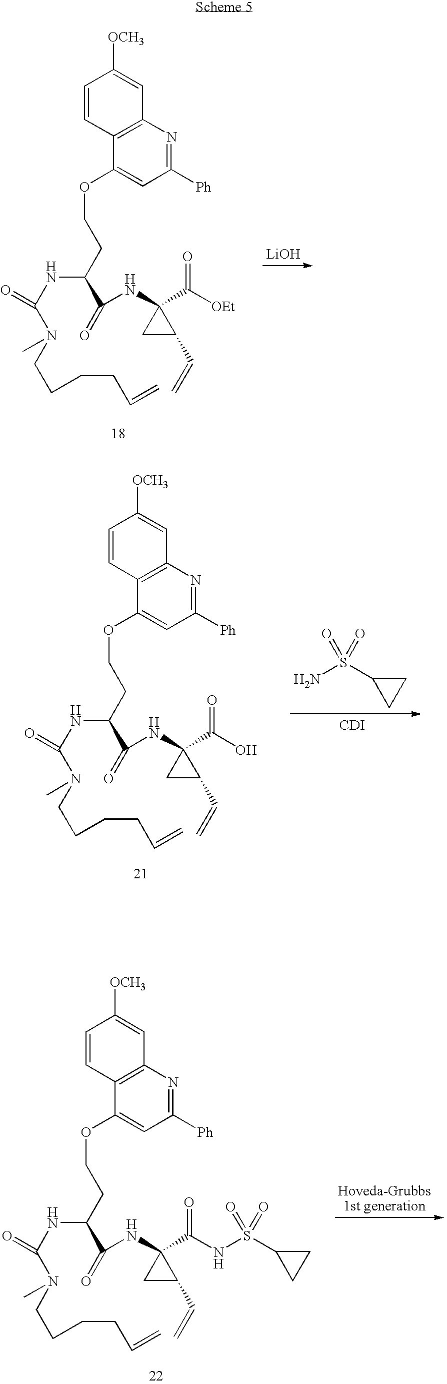

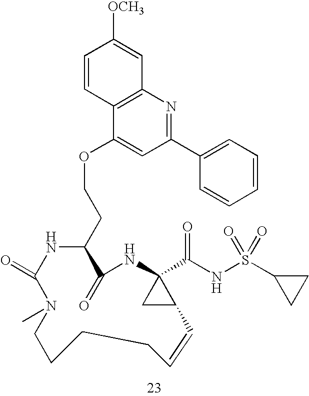

Step 2: Compound 21 (153 mg, 0.25 mmol) and carbonyldiimidazole (82 mg, 0.51 mmol) were heated at 80° C. under N₂ in a microwave apparatus for 20 min. A TLC (EtOAc) of the crude revealed disappearance of the starting material and a LC/MS analysis showed the presence of the intermediate oxazolinone. Cyclopropylsulfonamide (62 mg, 0.51 mmol) and DBU (76 μL, 0.51 mmol) were added and the mixture heated at 70° C. under N₂ in a microwave apparatus for another 20 min. The solvents were evaporated in vacuo and the residue was redissolved in dichloromethane. The organic phase was washed with 0.05 N HCl, brine, and evaporated under vacuum. The crude residue was purified by column chromatography on silica gel with DCM/MeOH (98:2 to 90:10, v/v) to afford compound 22 (97 mg) as a yellow solid.

¹H NMR (DMSO-d₆, 400.13 MHz) δ 0.98-1.08 (m, 4H), 1.18-1.29 (m, 3H), 1.34-1.41 (m, 2H), 1.60-1.69 (m, 1H), 1.90-1.95 (m, 2H), 2.10-2.32 (m, 3H), 2.80 (s, 3H), 2.85-2.95 (m, 1H), 3.15-3.25 (m, 2H), 3.92 (s, 3H), 4.06-4.19 (m, 1H), 4.33-4.47 (m, 2H), 4.82-4.93 (m, 2H), 5.03-5.12 (m, 1H), 5.16-5.27 (m, 1H), 5.54-5.77 (m, 2H), 6.70 (br, 1H), 7.16 (dd, J=9.2 and 2.6 Hz, 1H), 7.37 (s, 2H), 7.46-7.54 (m, 3H), 8.10 (dd, J=9.2 and 5.6 Hz, 1H), 8.24 (d, J=6.8 Hz, 2H), 8.87 (br, 1H), 10.92 (br, 1H); MS (ESI, El⁻) m/z=703 (M-H).

Step 3: Compound 22 (53 mg, 0.075 mmol) and Hoveyda-Grubbs second generation (9 mg, 20% mol) in degassed DCM (50 mL) were stirred under N₂. The solution was divided in 5 batches of 10 mL of solution and heated under microwave at 100° C. for 1 hr. After reaction, all batches were mixed together. The solvent was evaporated and the crude residue purified twice by semi-preparative HPLC (C18) to give macrocycle 23 as a slight yellow solid (4 mg).

¹H NMR (CD₃COCD₃, 400.13 MHz) δ 0.89-1.85 (m, 14H), 2.29-2.64 (m, 3H), 2.98 (s, 3H), 3.99 (s, 3H), 4.37-4.61 (m, 4H), 4.98-5.04 (m, 1H), 5.62-5.72 (m, 1H), 5.76-5.84 (m, 1H), 7.18 (d, J=9.2 Hz, 1H), 7.42-7.55 (m, 5H), 8.12-8.17 (m, 1H), 8.28-8.37 (m, 3H), 11.51 (br, 1H); MS (ESI, El⁺) m/z=676 (MH⁺).

### Example 4

### Preparation of Macrocyclic Compound 30

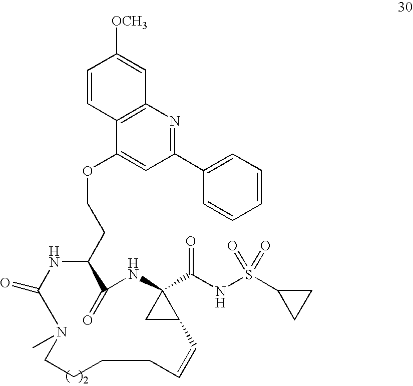

The synthesis of macrocyclic compound 30 is shown in Scheme 6

Step 1: A solution of compound 15 (1.046 g, 1.95 mmol), N-methylhept-6-en-1-amine 12b (Ts salt, 0.700 g, 2.34 mmol), and TEA (0.330 mL, 2.34 mmol) was refluxed overnight under N₂. After cooling to RT, the solvents were evaporated in vacuo and the crude residue was purified by column chromatography on silica gel with PE/EtOAc (93:7 to 40:60, v/v) to afford compound 25 as a white solid (0.804 g, 69% yield).

¹H NMR (CDCl₃, 400.13 MHz) δ 1.22-1.29 (m, 2H), 1.33-1.40 (m, 2H), 1.47-1.54 (m, 2H), 2.02 (m, 2H), 2.43-2.63 (m, 2H), 2.89 (s, 3H), 3.19-3.27 (m, 2H), 4.02 (s, 3H), 4.42 (m, 2H), 4.82 (dd, J=6.7 and 12.6 Hz, 1H), 4.90-4.95 (m, 2H), 5.15 (d, J=12.3 Hz, 1H), 5.18 (d, J=12.3 Hz, 1H), 5.25 (d, J=6.80 Hz, 1H), 5.71-5.81 (m, 1H), 6.98 (s, 1H), 7.16 (dd, J=9.2 and 2.4 Hz, 1H), 7.27 (m, 5H), 7.51-7.57 (m, 3H), 7.7.50-7.52 (m, 3H), 8.03 (d, J=9.20 Hz, 1H), 8.12 (m, 2H); MS (ESI, El⁺) m/z=596 (MH⁺).

Step 2: A solution of compound 25 (0.804 g, 1.41 mmol) and LiOH (0.102 g, 4.20 mmol) in a mixture of water (20 mL) and THF (20 mL) was stirred overnight at room temperature. The volatile solvent was then evaporated and the aqueous layer was acidified to pH=3-4 with a solution of 1M HCl. After extraction three times with EtOAc, the combined organic layers were dried (Na₂SO₄) and concentrated in vacuo to afford the acid 26 as a white solid, which was used in the next step without further purification.

MS (ESI, El⁺) m/z=506 (MH⁺).

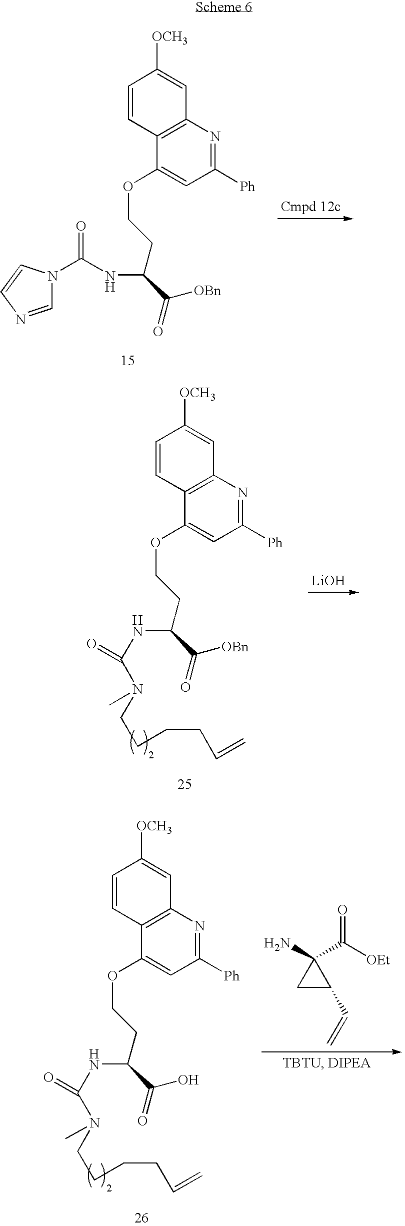

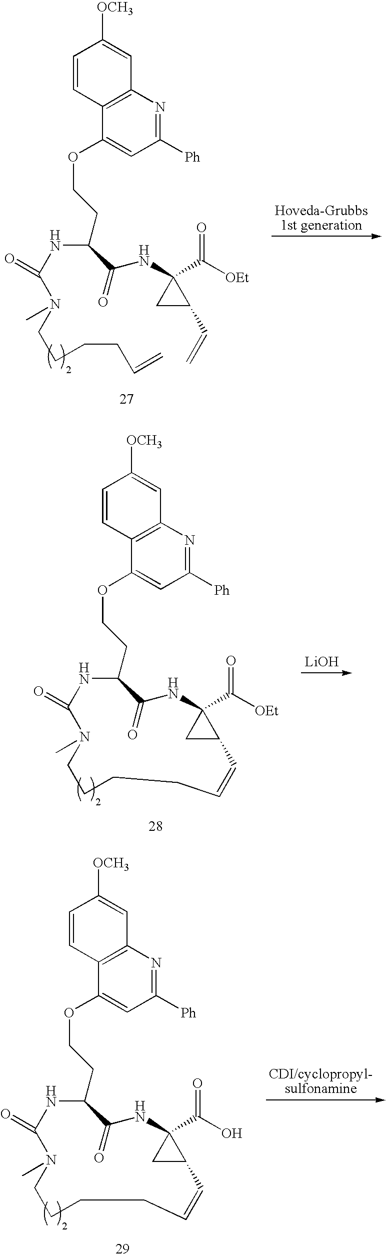

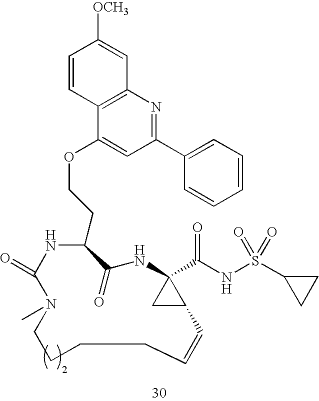

Step 3: To a solution of compound 26 (1.41 mmol), (1R,2S)-ethyl 1-amino-2-vinylcyclopropanecarboxylate (Ts salt, 0.461 g, 1.41 mmol), and DIPEA (0.495 mL, 2.82 mmol) in anhydrous DMF (20 mL) under N₂ was added TBTU (0.947 g, 1.41 mmol). The reaction mixture was stirred at RT overnight. DMF was then evaporated under vacuum, water was added and the aqueous layer extracted three times with EtOAc. The combined organic layers were washed with brine, then dried and concentrated in vacuo. The crude residue was purified by column chromatography on silica gel with PE/EtOAc (1:1, v/v) to afford compound 27 (0.587 g, 65% yield θ from 25) as an off-white solid.

¹H NMR (DMSO, 400.13 MHz) δ 1.07-1.35 (m, 10H), 1.62 & 1.67 (2dd, J=5.1 and 7.7 Hz, 1H, 2 rotamers in 54/46 ratio), 1.88 (m, 2H), 2.10-2.38 (m, 3H), 2.75 & 2.77 (2s, 3H, 2 rotamers), 3.12 (m, 2H), 3.91 (s, 3H), 3.96-4.06 (m, 2H), 4.41 (m, 3H), 4.84-4.92 (m, 2H), 5.07-5.11 (m, 1H), 5.24-5.30 (m, 1H), 5.56-5.73 (m, 2H), 6.21 (dd, J=11.7 and 8.2 Hz, 1H), 7.16 (dd, J=9.1 and 2.4 Hz, 1H), 7.36 & 7.37 (2s, 2H, 2 rotamers), 7.45-7.53 (m, 3H), 8.09 & 8.11 (2d, J=8.7 Hz, 1H), 8.23 (d, J=6.9 Hz, 2H), 8.75 & 8.83 (2s, 1H, 2 rotamers); MS (ESI, El⁺) m/z 643 (MH⁺).

Step 4: Compound 27 (580 mg, 0.90 mmol) and Hoveyda-Grubbs 1st generation (108 mg, 20% mol) were refluxed overnight under N₂ in previously degassed DCM (560 mL, 0.016M). After cooling, the solvent was evaporated and the crude residue was purified by column chromatography on silica gel with PE/EtOAc (1:1, v/v) to afford macrocycle 28 (355 mg) as a mixture of cis and trans epimers ((1R,2R) on cyclopropane). Pure 28 was isolated by semi-preparative HPLC (C18) as a white solid.

¹H NMR (CDCl₃, 400.13 MHz) δ 1.18 (t, J=7.0 Hz, 3H), 1.13-1.27 (m, 3H), 1.30-1.53 (m, 5H), 1.90 (dd, J=5.6 and 7.9 Hz, 1H), 2.16 (m, 3H), 2.26-2.36 (m, 1H), 2.50-2.58 (m, 1H), 2.76-2.81 (m, 2H), 2.86 (s, 3H), 3.94 (s, 3H), 3.98 (m, 1H), 4.11 (q, J=7.0 Hz, 2H), 4.37-4.50 (m, 2H), 4.79-4.85 (m, 1H), 5.15 (d, J=9.4 Hz, 1H), 5.21 (t, J=10.0 Hz, 1H), 5.58 (m, 1H), 7.09 (s, 2H), 7.41-7.43 (m, 2H), 7.45-7.51 (m, 3H), 8.06 (d, J=8.6 Hz, 1H); MS (ESI, El⁺) m/z=615 (MH⁺).

Step 5: A solution of compound 28 (186 mg, 0.30 mmol) and LiOH (40 mg, 1.5 mmol) in a mixture of water (10 mL) and THF (10 mL) was stirred overnight at room temperature. Another 5 eq. of LiOH was added and the reaction media was stirred overnight. THF was then evaporated and the aqueous layer acidified to pH=4-5 with a solution of 1M HCl. After extraction three times with dichloromethane, the combined organic layers were dried (Na₂SO₄) and concentrated in vacuo to afford the acid 29 as a grey solid, which was used in the next step without further purification (154 mg). A pure sample was obtained by semi-preparative HPLC (C18) to afford pure 29 as a white solid.

MS (ESI, El⁺) m/z=587 (MH⁺).

Step 6: Compound 29 (111 mg, 0.19 mmol) and carbonyldiimidazole (61 mg, 0.38 mmol) were heated at 80° C. under N₂ in a microwave apparatus for 20 min. A TLC (EtOAc) of the crude revealed disappearance of the starting material and a LC/MS analysis showed the presence of the intermediate oxazolinone. Cyclopropylsulfonamide (46 mg, 0.38 mmol) and DBU (56 μL, 0.38 mmol) were added and the mixture heated at 80° C. under N₂ in a microwave apparatus for another 20 min. The solvents were evaporated in vacuo and the residue was redissolved in DCM. The crude residue was filtrated on a silica gel pad eluted with DCM/MeOH (95:5, v/v) and fractions containing compound were combined, evaporated under vacuum and further purified by semi-preparative HPLC to afford a pure fraction of compound 30 (15 mg) as a white solid.

MS (ESI, El⁺) m/z=690 (MH⁺).

### Example 5

### Preparation of Macrocyclic Compound 41

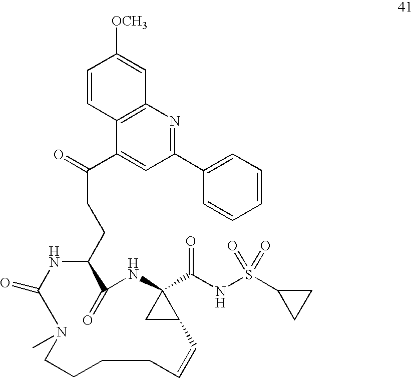

The synthesis of macrocyclic compound 41 is shown in Scheme 7

Step 1: Under inert atmosphere, O-tert-butyldimethylsilyl-L-serine methyl ester (29.8 g, 127.9 mmol) was added via a canula to a solution of carbonyldiimidazole (23.85 g, 147 mmol) in dry DCM (250 mL). The mixture was stirred for 15 hr and the reaction was monitored with TLC (CH₂Cl₂/MeOH, 90:3 (v/v), R_{f}: ˜0.4 for the starting material, and ˜0.6 for the desired product (U.V. visible). Solvents were evaporated and the crude residue was purified by chromatography on silica gel with PE/EtOAc (100:0 to 30:70, v/v) to afford compound 31 as a thick oil (17 g).

¹H NMR (CDCl₃, 400.13 MHz) δ 0.05 (s, 6H), 0.88 (s, 9H), 3.81 (s, 3H), 3.98 (dd, J=10.28 and 3.08 Hz, 1H), 4.16 (dd, J=10.28 and 2.52 Hz, 1H), 4.68 (dt, J=7.76 and 3.00 Hz, 1H), 6.49 (d, J=7.76 Hz, 1H), 7.13 (s, 1H), 7.35 (s, 1H), 8.14 (s, 1H).

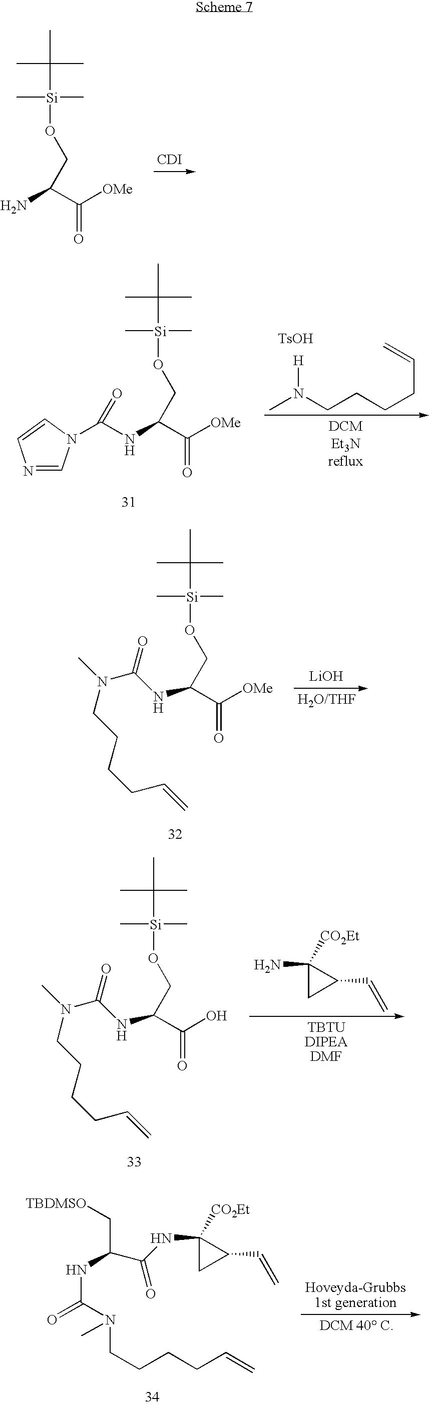

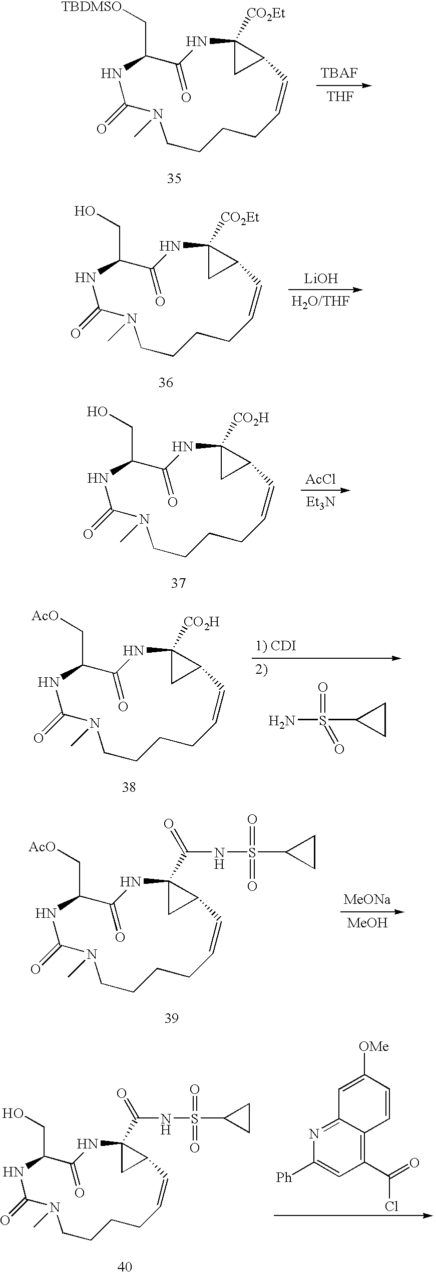

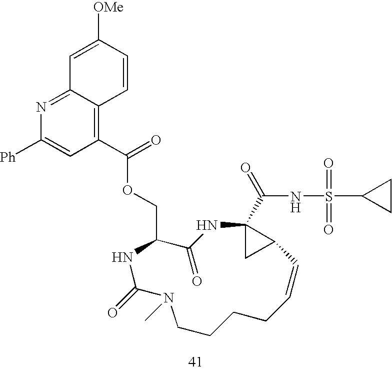

Step 2: A solution of compound 31 (17.0 g, 51.9 mmol), N-methylhex-5-en-1-amine (Ts salt, 17.81, 62.3 mmol)(compound 12a), and TEA (6.9 mL, 67.5 mmol) was heated at 40° C. for 2 hr under N₂ and the reaction was monitored by TLC (PE/EtOAc, 5:5 (v/v), R_{f}: ˜0.7 for the desired product and ˜0.3 for the starting material 31. After cooling to RT, the organic layer was washed with water, dried (Na₂SO₄), and concentrated in vacuo. The crude residue was purified by column chromatography on silica gel with PE/EtOAc (1:0 to 7:3, v/v) to afford compound 32 as thick oil (15.0 g).

¹H NMR (CDCl₃, 400.13 MHz) δ 0.02 (s, 3H), 0.04 (s, 3H), 0.87 (s, 9H), 1.40 (m, 2H), 1.58 (m, 2H), 2.09 (m, 2H), 2.92 (s, 3H), 3.27 (t, J=6.64 Hz, 3H), 3.74 (s, 3H), 4.00 (dd, J=9.44 and 3.00 Hz, 1H), 4.22 (dd, J=9.44 and 2.44 Hz, 1H), 4.68 (dt, J=7.84 and 2.80 Hz, 1H), 5.07 (d, J=9.60 Hz, 1H), 5.12 (d, J=16.2 Hz, 1H), 5.24 (d, J=7.84), 5.80 (m, 1H).

Step 3: A solution of compound 32 (15.0 g, 40.26 mmol) and LiOH (2.9 g, 120.8 mmol) in a mixture of water (75 mL), THF (100 mL), and MeOH (40 mL) was stirred overnight at RT and the reaction was monitored by TLC (DCM/MeOH, 90:10 (v/v), R_{f}: ˜0.15 for the desired product). The volatile solvents were evaporated at 0° C. and the aqueous layer was acidified to pH=3-4 with a solution of 1M HCl (170 mL). The aqueous solution was extracted three times with EtOAc. The combined organic layers were washed with water, dried (Na₂SO₄), and concentrated in vacuo to afford acid 33 as yellow oil (14.33 g), which was used in the next step without further purification.

¹H NMR (CDCl₃, 400.13 MHz) δ 0.07 (s, 6H), 0.88 (s, 9H), 1.40 (m, 2H), 1.58 (m, 2H), 2.06 (m, 2H), 2.92 (s, 3H), 3.27 (t, J=7.2 Hz, 3H), 3.86 (dd, J=10.16 and 4.28 Hz, 1H), 4.10 (dd, J=10.16 and 3.00 Hz, 1H), 4.44 (dt, J=6.76 and 3.20 Hz, 1H), 4.96 (d, J=10.16 Hz, 1H), 5.02 (d, J=17.12 Hz, 1H), 5.24 (d, J=6.76), 5.78 (m, 1H).

Step 4: To a solution of compound 33 (6.0 g, 16.7 mmol), (1R,2S)-ethyl 1-amino-2-vinylcyclopropanecarboxylate (Ts salt, 5.44 g, 16.7 mmol), DIPEA (8.8 mL, 50.1 mmol) in anhydrous DMF (40 mL) under N₂ was added TBTU (5.91 g, 18.4 mmol). The reaction mixture was stirred at RT overnight. The reaction mixture was diluted with EtOAc and washed with water. The organic layer was dried (Na₂SO₄) and concentrated in vacuo. The crude residue was purified by column chromatography on silica gel with PE/EtOAc (100:0 to 70:30. v/v) to afford compound 34 (4.8 g) as thick yellow oil.

MS (ESI, El⁺) m/z=496 (MH⁺).

Step 5: The diene 34 (500 mg, 1.01 mmol) was dissolved in 1,2-dichloroethane (102 mL, 0.01M) and degassed for 1 hr with a N₂ stream. The catalyst (Hoveyda-Grubbs 1^{st} generation, 30 mg, 5% mol) was then added and the mixture was stirred at 70° C. for 16 hr. After cooling, the reaction mixture was filtered on a small pad of silica and the filtrate was then evaporated. The crude residue was purified by column chromatography on silica gel with PE/EtOAc (1:0 to 1:1. v/v) to afford compound 35 (227 mg) as a yellow solid.

MS (ESI, El⁺) m/z=468 (MH⁺).

Step 6: Macrocycle 35 (227 mg, 0.49 mmol) was dissolved in THF (2 mL). Tetrabutylammonium fluoride (1M, in THF, 1.95 mL) was then added under inert atmosphere at RT. The mixture was stirred at RT for 2 hr. The solvent was evaporated and the crude residue was purified by column chromatography on silica gel with DCM/MeOH (100:0 to 100:5, v/v) to afford compound 36 (128 mg) as a white solid.

MS (ESI, El⁺) m/z=354 (MH⁺).

Step 7: LiOH (8.4 mg, 0.35 mmol) was added to a solution of macrocycle 36 (124 mg, 0.35 mmol) in a mixture of MeOH and H₂O (6 and 2 mL). The mixture was stirred at RT for 2 hr, and then 2 extra eq. of LiOH were added. The reaction mixture was stirred at RT overnight. The solvents were evaporated, and the mixture was acidified by addition of HCl (6M) and then concentrated. Purification by column chromatography on silica gel with DCM/MeOH/AcOH (95:5:1, v/v/v) afforded compound 37 (70 mg) as a white solid.

¹H NMR (CDCl₃, 400.13 MHz) δ 1.26-1.36 (m, 4H), 1.59-1.63 (m, 2H), 1.77-1.83 (m, 2H), 2.37 (q, J=8.9 Hz, 1H), 2.61-2.72 (m, 2H), 2.84 (s, 3H), 3.79 (dd, J=11.7 and 5.0 Hz, 1H), 4.03 (dd, J=11.7 and 2.9 Hz, 1H), 4.25 (m, 1H), 4.44 (m, 1H), 5.29 (dd, 1H), 5.61-5.67 (m, 2H), 8.30 (brs, 1H); MS (ESI, El⁺) m/z=326 (MH⁺).

Step 8: At 0° C., under inert atmosphere, TEA (0.104 mL, 0.752 mmol) was added to compound 37 (70 mg, 0.258 mmol) in DCM (5 mL). Acetyl chloride (0.03 mL, 0.430 mmol) was then added. The mixture was stirred at RT for 3 hr, and then 5 equivalent of AcOH were added and solvents were evaporated. Purification by column chromatography on silica gel with DCM/MeOH/HCO₂H (100:4:1, v/v/v) afforded compound 38 (43 mg) as a white solid.

MS (ESI, El⁺) m/z=368 (MH⁺).

Step 9: Under inert atmosphere, carbonyldiimidazole (38 mg, 0.234 mmol) was added to compound 38 (43 mg, 0.117 mmol) in dry THF (5 mL). This mixture was stirred at 80° C. for 3 hr. The reaction was monitored by TLC (DCM/MeOH, 100:4 (v/v), R_{f}: 0.4 for the desired product). At RT, under inert atmosphere, cyclopropyl sulfanamide (28.4 mg, 0.234 mmol) and DBU (0.036 mL, 0.234 mmol) were added. The mixture was stirred at 60° C. for 3 hr. The solvents were evaporated and the crude residue was purified by column chromatography on silica gel with DCM/MeOH (100:0 to 95:5, v/v) to afford compound 39 (40 mg) as a white solid.

MS (ESI, El⁺) m/z=471 (MH⁺).

Step 10: At 0° C., MeONa solid (4.6 mg, 0.025 mmol) was added to compound 39 (40 mg, 0.085 mmol) in dry MeOH (5 mL). This mixture was stirred at RT for 3 hr and then the solvent was evaporated. Purification by column chromatography on silica gel with DCM/MeOH (100:0 to 90:10, v/v) afforded compound 40 (24 mg) as a white solid.

MS (ESI, El⁺) m/z=429 (MH⁺).

Step 11: 7-Methoxy-2-phenylquinoline-4-carbonyl chloride (26 mg, 0.0895 mmol) in DCM (2 mL) was added to compound 40 (25 mg, 0.058 mmol) in solution of DCM (2 mL) and TEA (0.034 mL, 0.245 mmol) at 0° C. under N₂. The reaction mixture was stirred at RT for 16 hr, and then the solvents were evaporated. Purification by column chromatography on silica gel with DCM/MeOH (100:0 to 90:10, v/v) afforded compound 41 (13 mg) as a white solid.

¹H NMR (DMSO, 400.13 MHz) δ 0.88-1.74 (m, 13H), 2.74-2.86 (m, 6H), 3.97 (s, 3H), 4.21 (m, 1H), 4.31 (m, 1H), 4.63 (m, 1H), 4.77 (m, 1H), 5.13 (m, 1H), 5.58 (m, 1H), 6.58 (m, 1H), 7.35 (dd, J=9.3 and 2.4 Hz, 1H), 7.51-7.59 m (4H), 8.25 (m, 1H), 8.40 (s, 1H), 8.58 (d, J=8.9 Hz, 1H), 9.04 (brs, 1H), 11.21 (brs, 1H); MS (ESI, El⁺) m/z=690 (MH⁺).

### Example 6

### Preparation of Macrocyclic Compound 49

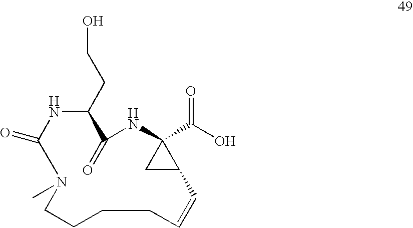

The synthesis of macrocyclic compound 49 is shown in Scheme 8

Step 1: Preparation of (S)—N-(2-oxotetrahydrofuran-3-yl)-1H-imidazole-1-carboxamide 42. To a stirred solution of CDI (14.7 g, 1.1 eq.), TEA (12.6 mL, 1.1 eq.) in DCM (160 mL) was added (S)-(−)-α-amino-γ-butyrolactone hydrobromide (15 g, 1 eq.) under nitrogen. The reaction mixture was stirred at room temperature for 3 hrs. The reaction mixture was concentrated and used directly in the next step without further purification.

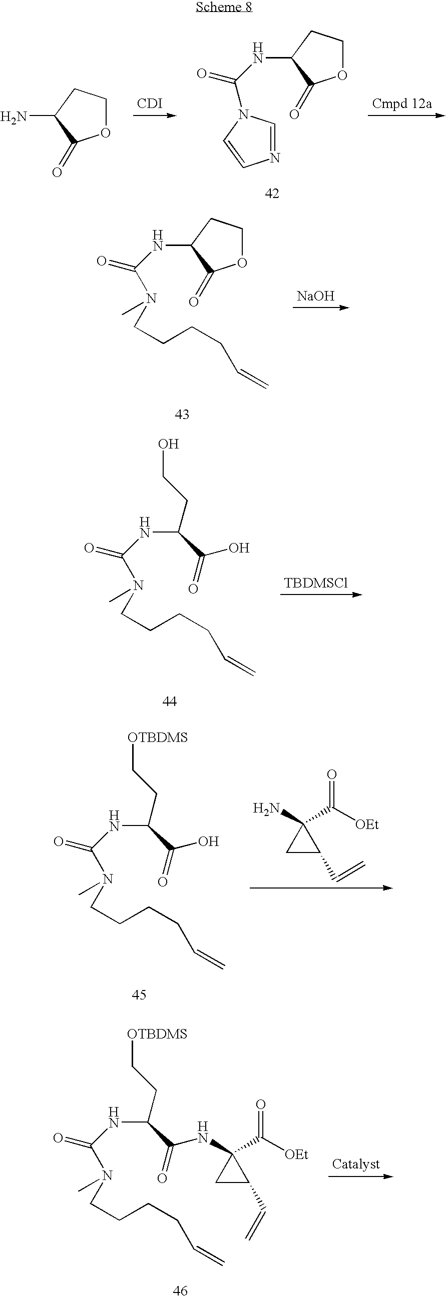

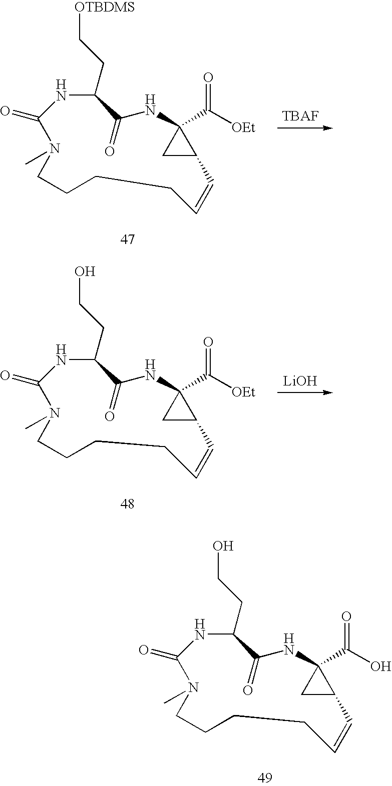

Step 2: Preparation of (S)-1-(hex-5-enyl)-1-methyl-3-(2-oxotetrahydrofuran-3-yl)urea 43. To compound 42 (82.4 mmol, 1 eq.) was added TEA (12.6 mL, 1.1 eq.) and compound 12a (26 g, 1.1 eq.) under nitrogen. The reaction mixture was refluxed for 16 hrs and cooled down to room temperature. The mixture was washed with water, dried over Na₂SO₄, filtered and concentrated under reduced pressure. The crude material was purified by flash chromatography on silica gel to yield compound 43 as a white solid in 95% yield.

¹H NMR (DMSO-d₆, 400 MHz) δ 1.25-1.32 (quint, J=7.73 Hz, 2H), 1.38-1.46 (quint, J=7.80 Hz, 2H), 1.99-2.04 (q, J=7.18 Hz, 2H), 2.16-2.24 (m, 1H), 2.27-2.32 (m, 1H), 2.75 (s, 3H), 3.13-3.16 (t, J=7.73 Hz, 2H), 4.13-4.19 (q, J=8.55 Hz, 1H), 4.27-4.33 (td, J=8.83 Hz and J=1.93 Hz, 1H), 4.33-4.40 (q, J=8.73 Hz, 1H), 4.91-5.02 (m, 2H), 5.75-5.83 (m, 1H), 6.77 (d, J=8.00 Hz, 1H).

Step 3: Preparation of (S)-2-(3-(hex-5-enyl)-3-methylureido)-4-hydroxybutanoic acid 44. To a stirred solution of compound 43 (22 g, 1 eq.) in methanol (120 mL) was added 1N NaOH (125 mL). The reaction mixture was stirred at room temperature for 2 hrs. Solvent was removed under reduced pressure. The mixture was acidified to pH 4 with 1N HCl, saturated with NaCl, and extracted with EtOAc. The aqueous phase was acidified (pH 3) and extracted with EtOAc. Combined organics were dried over Na₂SO₄, filtered, and concentrated under reduced pressure to yield compound 44 as yellow oil in 95% yield.

¹H NMR (DMSO-d₆, 400 MHz) δ 1.25-1.32 (quint, J=7.73 Hz, 2H), 1.38-1.46 (quint, J=7.80 Hz, 2H), 1.72-1.83 (m, 2H), 1.98-2.04 (q, J=7.37 Hz, 2H), 2.76 (s, 3H), 3.09-3.22 (m, 2H), 3.39-3.50 (m, 2H), 4.09-4.15 (m, 1H), 4.91-5.02 (m, 2H), 5.72-5.82 (m, 1H), 6.25 (d, J=7.77 Hz, 1H).

Step 4: Preparation of (S)-4-(tert-butyldimethylsilyloxy)-2-(3-(hex-5-enyl)-3-methylureido)butanoic acid 45. To a stirred solution of compound 44 (23 g, 1 eq.) in DCM (250 mL) was added t-butyldimethylsilylchloride (26 g, 2 eq.) and TEA (24 mL, 2 eq.) at 0° C. The reaction mixture was allowed to warm up to room temperature and was stirred for 72 hrs. Solvent was evaporated and water was added. The mixture was extracted with EtOAc, dried over Na₂SO₄, filtered, and concentrated under reduced pressure to yield compound 45 as orange oil in 87% yield.

¹H NMR (DMSO-d₆, 400 MHz) δ 0.18 (s, 3H), 0.20 (s, 3H), 0.88 (s, 9H), 1.25-1.32 (quint, J=7.69 Hz, 2H), 1.38-1.45 (quint, J=7.70 Hz, 2H), 1.79-1.81 (m, 2H), 1.99-2.03 (q, J=7.00 Hz, 2H), 2.76 (s, 3H), 3.13-3.17 (m, 2H), 3.61-3.65 (m, 2H), 4.14-4.19 (m, 1H), 4.90-5.00 (m, 2H), 5.73-5.81 (m, 1H), 6.26 (d, J=7.69 Hz, 1H).

Step 5: Preparation of (1R,2S)-ethyl 1-((S)-4-(tert-butyldimethylsilyloxy)-2-(3-(hex-5-enyl)-3-methylureido)butanamido)-2-vinylcyclopropanecarboxylate 46. To a stirred solution of compound 45 (7.55 g, 1 eq.) in anhydrous DMF (250 mL) was added (1R, 2S) ethyl-1-amino-2-vinylcyclopropane-carboxylate tosylate salt (7) (5 g, 1.1 eq.), TBTU (5 g, 1.1 eq.), and DIPEA (7.3 mL, 3 eq.) at 0° C. The reaction mixture was then allowed to warm up to room temperature and stirred for 16 hrs. Water was added and the mixture was extracted thrice with EtOAc. Organics were dried over Na₂SO₄, filtered, and concentrated under reduced pressure. The crude material was purified by flash chromatography on silica gel to yield compound 46 as yellow oil in 76% yield.

¹H NMR (DMSO-d₆, 400 MHz) δ 0.002 (s, 6H), 0.84 (s, 9H), 1.10-1.16 (t, 7.02 Hz, 3H), 1.18-1.22 (m, 1H), 1.26-1.32 (m, 2H), 1.38-1.45 (m, 2H), 1.59-1.62 (td, J=6.61 and 2.64 Hz, 1H), 1.70-1.88 (m, 2H), 1.98-2.03 (q, J=7.03 Hz, 2H), 2.06-2.14 (q, J=8.90 Hz, 1H), 2.78 (s, 3H), 3.14-3.19 (t, J=7.03 Hz, 2H), 3.58-3.62 (t, J=6.40 Hz, 2H), 3.95-4.05 (m, 2H), 4.09-4.15 (m, 1H), 4.91-5.00 (m, 2H), 5.05-5.28 (m, 2H), 5.56-5.65 (m, 1H), 5.73-5.82 (m, 1H), 6.00 (d, J=7.50 Hz, 1H), 8.51 (s, 1H); MS (ESI, EI⁻) m/z=508 (MH⁻).

Step 6: Preparation of (1R,4S,14S,Z)-ethyl 4-(2-(tert-butyldimethylsilyloxy)ethyl)-7-methyl-3,6-dioxo-2,5,7-triazabicyclo[12.1.0]pentadec-12-ene-1-carboxylate 47. A solution of compound 46 (1 g, 1 eq.) in dry DCM (1 L) was degassed by bubbling nitrogen for 30 min. The Hoveyda-Grubbs 1^{st} generation catalyst (20% mol.) was added and the reaction mixture was refluxed under nitrogen for 1 day. The mixture was then filtered and concentrated in vacuum. The residue was purified by chromatography to give compound 47 as a pale brown solid in 50% yield.

MS (ESI, EI⁺) m/z=482 (MH⁺).

Step 7: Preparation of (1R,4S,14S,Z)-ethyl 4-(2-hydroxyethyl)-7-methyl-3,6-dioxo-2,5,7-triazabicyclo[12.1.0]pentadec-12-ene-1-carboxylate 48. To a stirred solution of compound 47 (784 mg, 1 eq.) in anhydrous THF (4 mL) was added TBAF (IN) in THF (3.92 mL, 2 eq.) at room temperature. The reaction mixture was stirred at room temperature for 2 hrs. Solvent was evaporated. The residue was dissolved with EtOAc, washed with brine, dried over Na₂SO₄, filtered, and concentrated under reduced pressure. The crude material was purified by flash chromatography on silica gel to yield compound 48 as a brown solid in 44% yield.

¹H NMR (DMSO-d₆, 400 MHz) δ 0.9-1.01 (m, 2H), 1.10-1.16 (t, 7.02 Hz, 3H), 1.32-1.36 (m, 2H), 1.38-1.46 (m, 2H), 1.63-1.67 (m, 1H), 1.69-1.74 (m, 3H), 2.38-2.45 (q, J=9.34 Hz, 1H), 2.62-2.68 (m, 1H), 2.75 (s, 3H), 3.49-3.53 (q, J=6.02 Hz, 2H), 3.95-4.00 (q, J=7.02 Hz, 2H), 4.16-4.22 (t, J=13.11 Hz, 1H), 4.52-4.54 (t, J=5.02 Hz, 1H), 5.41-5.51 (m, 2H), 5.95 (d, J=8.03 Hz, 1H), 8.20 (s, 1H); MS (ESI, EI⁻) m/z=366 (MH⁻).

Step 8: Preparation of (1R,4S,14S,Z)-4-(2-hydroxyethyl)-7-methyl-3,6-dioxo-2,5,7-triazabicyclo[12.1.0]pentadec-12-ene-1-carboxylic acid 49. Compound 48 (166 mg, 1 eq.) and LiOH (76 mg, 7 eq.) in water (5 mL) and THF (5 mL) were stirred at room temperature for 16 hrs. The reaction mixture was acidified with 1N HCl to pH 4. Solvent was evaporated. The mixture was extracted with EtOAc, dried over Na₂SO₄, filtered, and concentrated under reduced pressure to yield compound 49 as a white solid in 91% yield.

¹H NMR (CDCl₃, 400 MHz): δ 1.23-1.29 (m, 6H), 1.57-1.61 (m, 2H), 1.76-1.80 (m, 2H), 1.92-2.01 (m, 2H), 2.65-2.70 (m, 2H), 2.82 (s, 3H), 3.81 (t, J=5.19 Hz, 2H), 4.39-4.43 (m, 2H), 5.22 (t, J=9.90 Hz, 1H), 5.45 (d, J=8.02 Hz, 1H), 5.61-5.67 (m, 1H), 8.03 (brs, 1H);

MS (ESI, EI⁺) m/z=340 (MH⁺).

### Example 7

### Preparation of Cyclopropanesulfonic Acids 52

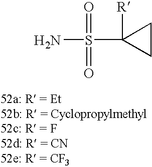

The synthesis of macrocyclic compound 52 is shown in Scheme 9, where R′ in compound 51 is the same as compound 52.

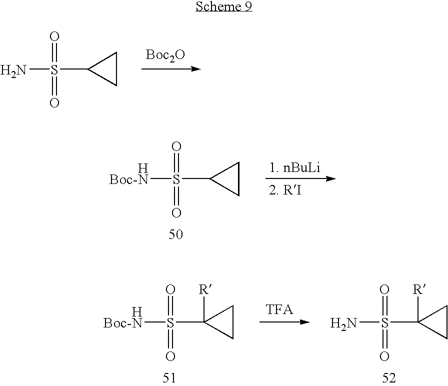

Step 1: Preparation of Boc-cyclopropane sulfonyl amide 50. To a solution of cyclopropanesulfonamide, TEA (13.9 mL), and DMAP (1.11 g, 0.1 eq.) (10.72 g, 1 eq.) in DCM (160 mL) was added a solution of Boc₂O (21.88 g, 0.8 eq.) in DCM (100 mL) was added dropwise at 0° C. over 30 min. The mixture was then allowed to warm up to room temperature and stirred for 3 hrs. The solution was washed with 1N HCl, water and brine, dried over Na₂SO₄, filtered, and concentrated under reduced pressure to yield after trituration in hexane compound 50 as a white solid in 87% yield.

¹H NMR (CDCl₃, 400 MHz): δ 0.92-0.96 (td, J=6.5 and 1.50 Hz, 2H), 1.51 (s, 9H), 1.60-1.64 (td, J=6.46 and 1.50 Hz, 2H), 1.25 (m, 1H), 6.99 (brs, 1H).

Step 2: Preparation of Boc-1-ethyl-cyclopropane sulfonyl amide 51a. To a stirred solution of compound 50 (15 g, 1 eq.) in THF (150 mL) was added dropwise nBuLi (68 mL, 2.5 eq.) at −80° C. The mixture was stirred at −80° C. for 10 min and ethyl iodide (8.13 mL, 1.5 eq.) was added. The temperature was allowed to rise up to −30° C. Dry ethanol (50 mL) was added, followed by water, and then acidified with 1N HCl to pH 6. The mixture was concentrated, extracted with EtOAc, washed with brine, dried with Na₂SO₄, filtered, concentrated under reduced pressure, and purified by chromatography on silica gel to yield compound 51a as a yellow solid in 45% yield.

¹H NMR (CDCl₃, 400 MHz): δ 0.92-0.96 (td, J=6.5 and 1.50 Hz, 2H), 1.04 (t, J=7.40 Hz, 3H), 1.51 (s, 9H), 1.60-1.64 (td, J=6.46 and 1.50 Hz, 2H), 1.95 (q, J=7.49 Hz, 2H), 6.99 (brs, 1H).

Step 3: Preparation of Boc-1-cyclopropylmethyl-cyclopropanesulfonic acid methylamide 51b. Compound 51b was synthesized from compound 50 (6.74 g, 1 eq.) and (methylbromo)cyclopropane (4.44 mL, 1.5 eq.) as a beige solid in 40% yield, following the procedure as described for compound 51a.

¹H NMR (CDCl₃, 400 MHz): δ 0.16-0.20 (m, 2H), 0.52-0.54 (m, 2H), 0.74-0.78 (m, 1H), 1.02-1.05 (td, J=6.00 and 1.60 Hz, 2H), 1.36-1.39 (td, J=6.00 and 1.60 Hz, 2H), 1.43 (s, 9H), 1.89 (d, J=6.80 Hz, 2H), 7.02 (brs, 1H).

Step 4: Preparation of Boc-1-fluoro-cyclopropane sulfonyl amide 51c. Compound 51c was synthesized from compound 50 and NFSi as a yellow solid in 50% yield, following the procedure as described for compound 51a.

¹H NMR (CDCl₃, 400 MHz) δ 0.98 (s, 9H), 1.45-1.62 (m, 4H), 4.85 (brs, 2H).

Step 5: Preparation of Boc-1-cyano-cyclopropane sulfonyl amide 51d. Compound 51d was synthesized from compound 50 and p-toluene sulfonyl cyanide as a yellow solid in 50% yield, following the procedure as described for compound 51a.

¹H NMR (CDCl₃, 400 MHz) δ 1.02 (s, 9H), 1.70-1.73 (m, 2H), 1.81-1.84 (m, 2H), 5.05 (brs, 2H).

Step 6: Preparation of Boc-1-trifluoromethyl-cyclopropane sulfonyl amide 51e. Compound 51e was synthesized from compound 50 and trifluoromethyl iodide as a yellow solid in 47% yield, following the procedure as described for compound 51a.

¹H NMR (CDCl₃, 400 MHz) δ 1.40-1.44 (m, 2H), 1.54 (s, 9H), 2.02-2.06 (m, 2H), 7.63 (brs, 1H).

Step 7: Preparation of 1-ethyl-cyclopropane sulfonyl amide 52a. Compound 51a (7.70 g, 1 eq.) and TFA (16 mL) in DCM (60 mL) were stirred at room temperature for 16 hrs. The reaction mixture was concentrated under reduced pressure and purified by elution on silica cake (DCM/MeOH, 9/1) to yield compound 52a as a yellow powder in quantitative yield.

¹H NMR (CDCl₃, 400 MHz): δ 0.86-0.88 (td, J=6.46 and 1.50 Hz, 2H), 1.04 (t, J=7.40 Hz, 3H), 1.36 (t, J=6.02 Hz, 2H), 1.95 (q, J=7.40 Hz, 2H), 4.59 (brs, 2H).

Step 8: Preparation of 1-cyclopropylmethyl-cyclopropanesulfonic acid methylamide 52b. Compound 52b was synthesized from compound 51b (500 mg, 1 eq.) as a white solid in 90% yield, following the procedure as described for compound 52b.

¹H NMR (CDCl₃, 400 MHz): δ 0.16-0.20 (m, 2H), 0.52-0.54 (m, 2H), 0.74-0.78 (m, 1H), 1.02-1.05 (td, J=6.00 and 1.60 Hz, 2H), 1.36-1.39 (td, J=6.00 and 1.60 Hz, 2H), 1.89 (d, J=6.80 Hz, 2H), 4.72 (brs, 2H).

Step 9: Preparation of 1-fluoro-cyclopropane sulfonyl amide 52c. Compound 52c was synthesized from compound 51c as a white solid in 25% yield, following the procedure as described for compound 52a.

¹H NMR (CDCl₃, 400 MHz) δ 1.45-1.62 (m, 4H), 4.85 (brs, 2H).

Step 10: Preparation of 1-cyano-cyclopropane sulfonyl amide 52d. Compound 52d was synthesized from compound 51d as a white solid in 25% yield, following the procedure as described for compound 52a.

¹H NMR (CDCl₃, 400 MHz) δ 1.70-1.73 (m, 2H), 1.81-1.84 (m, 2H), 5.05 (brs, 2H).

Step 11: Preparation of 1-trifluoromethyl-cyclopropane sulfonyl amide 52e. Compound 52e was synthesized from compound 51e as a white solid in 96% yield, following the procedure as described for compound 52a.

¹H NMR (DMSO-d₆, 400 MHz) δ 1.20-1.23 (t, J=6.60 Hz, 2H), 1.49-1.52 (t, J=6.60 Hz, 2H), 7.13 (s, 2H).

### Example 8

### Preparation of Cyclopropanesulfonic Acids 52f

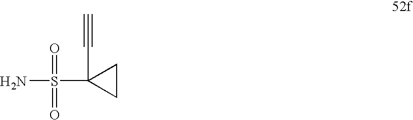

The synthesis of macrocyclic compound 52f is shown in Scheme 10.

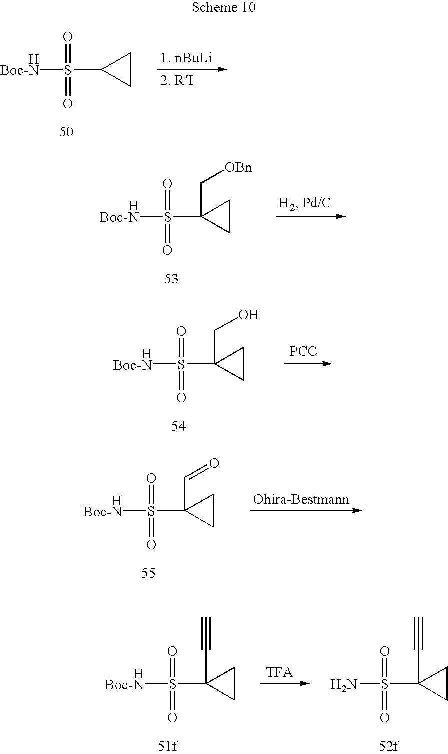

Step 1: Preparation of N-Boc-1-ethylbenzyloxy-cyclopropanesulfonic acid amide 53. To a stirred solution of compound 50 (500 mg, 2.26 mmol) in anhydrous THF (5 mL) was added nBuLi (2.26 mL, 5.65 mmol) dropwise at −80° C. The mixture was stirred at −80° C. for 10 min and then bromomethoxy-methylbenzene (271 μL, 3.39 mmol) was added dropwise at −80° C. The mixture was then allowed to warm up to −30° C. Water was then slowly added, followed by EtOAc. Organics were separated, dried over Na₂SO₄, filtered, concentrated under reduced pressure, and purified by chromatography on silica gel (EtOAc/DCM) to yield compound 53 as a white solid in 30% yield.

¹H NMR (CDCl₃, 400 MHz) δ 1.04 (td, J=1.72 and 6.40 Hz, 2H), 1.49 (s, 9H), 1.73 (td, J=1.72 and 6.40 Hz, 2H), 3.78 (s, 2H), 4.56 (s, 2H), 7.07 (brs, 1H), 7.30-7.38 (m, 5H).

Step 2: Preparation of N-Boc-1-(hydroxyethyl)-cyclopropanesulfonic acid amide 54. Compound 53 (2 g, 5.87 mmol) was reacted in a H-Cube® (Thales Technology) with a Pd/C 10% cartridge at 20 bars and 50° C. The crude material was purified by chromatography on silica gel (EtOAc/DCM) to yield compound 54 in 70% yield.

¹H NMR (CDCl₃, 400 MHz) δ 1.09 (t, J=6.32 Hz, 2H), 1.49 (s, 9H), 1.61 (t, J=6.32 Hz, 2H), 3.72 (s, 1H), 3.89 (s, 2H), 8.23 (brs, 1H).

Step 3: Preparation of N-Boc-1-formyl-cyclopropanesulfonic acid amide 55. To a stirred solution of compound 54 (100 mg, 0.39 mmol) in DCM (2 mL) was added pyridinium chlorochromate (130 mg, 0.60 mmol). The mixture was stirred at room temperature for 16 hrs and then filtered through a silica gel column with DCM to yield compound 54 after removal of solvent in 66% yield.

¹H NMR (CDCl₃, 400 MHz) δ 1.49 (s, 9H), 1.76 (m, 2H), 2.01 (m, 2H), 9.91 (s, 1H).

Step 4: Preparation of N-Boc-1-ethynyl-cyclopropanesulfonic acid amide 51f. To a stirred solution of compound 54 (230 mg, 0.92 mmol) in MeOH (5 mL) was added K₂CO₃ (255 mg, 1.84 mmol) and Ohira-Bestmann reagent (215 g, 1.10 mmol) at 0° C. The mixture was stirred at room temperature for 16 hrs and then concentrated under reduced pressure. Water, EtOAc, and citric acid (to pH 4-5) were added. Organics were separated, dried over Na₂SO₄, filtered, and concentrated under reduced pressure to yield compound 51f in 85% yield.

¹H NMR (CDCl₃, 400 MHz) δ 1.50 (m, 2H), 1.53 (s, 9H), 1.92 (m, 2H), 2.37 (s, 1H), 7.15 (brs, 1H).

Step 5: Preparation of 1-ethynyl-cyclopropanesulfonic acid amide 52f. A mixture of compound 51f (200 mg, 0.81 mmol) and TFA (0.3 mL) in DCM (5 mL) was stirred at room temperature for 16 hrs. The reaction mixture was concentrated under reduced pressure and the crude material was purified by chromatography on silica gel (MeOH/DCM) to yield compound 52f in 70% yield.

¹H NMR (CDCl₃, 400 MHz) δ 1.43 (td, J=2.90 and 4.80 Hz, 2H), 1.70 (td, J=2.90 and 4.80 Hz, 2H), 2.38 (s, 1H), 4.79 (s, 2H).

### Example 9

### Preparation of Cyclopropanesulfonic Acid 57

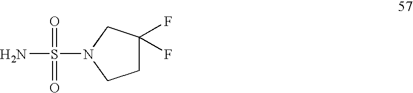

The synthesis of macrocyclic compound 57 is shown in Scheme 11.

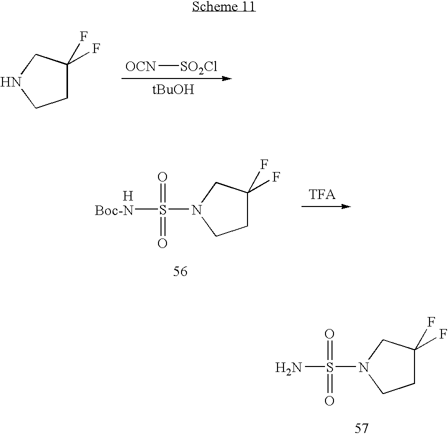

Step 1: Preparation of N-Boc-3,3-difluoro-pyrrolidine-1-sulfonic acid amide 56. To a stirred solution of tBuOH (135 μL, 1 eq.) in DCM (3 mL) was added dropwise chlorosulfonyl isocyanate (123 μL, 1 eq.) at 0° C. The reaction mixture was stirred at 0° C. for 30 min and 3,3-difluoropyrrolidine hydrochloride (223 mg, 1.1 eq.) was added, followed by TEA (431 μL, 2.2 eq.). The mixture was let to warm up to room temperature and stirred for 1 hr. The solution was diluted with DCM, washed with water, dried with Na₂SO₄, filtered, and concentrated under reduced pressure to yield after trituration in diethylether compound 56 as an off-white solid in 50% yield.

¹H NMR (CDCl₃, 400 MHz): δ 1.47 (s, 9H), 2.32-2.43 (m, 2H), 3.70-3.74 (t, J=7.25 Hz, 2H), 3.76-3.82 (t, J=12.70 Hz, 2H), 7.82 (brs, 1H).

Step 2: Preparation of 3,3-difluoro-pyrrolidine-1-sulfonic acid amide 57. Compound 57 was synthesized from compound 56 as a beige solid in 35% yield, following the procedure as described for compound 52a.

¹H NMR (CDCl₃, 400 MHz): δ 2.38-2.48 (m, 2H), 3.51-3.74 (t, J=7.25 Hz, 2H), 3.60-3.67 (t, J=12.70 Hz, 2H), 4.65 (brs, 2H); ¹⁹F NMR (CDCl₃, 285 MHz): δ −97.99 (s, 1F).

### Example 10

### Preparation of Cyclopropanesulfonic Acid 60

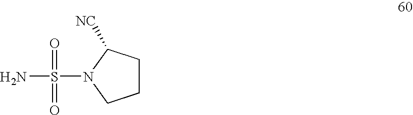

The synthesis of macrocyclic compound 60 is shown in Scheme 12. Compounds 58 and 59 were prepared following the procedure described in Organic Letters 2001, 3, 2241-2243.

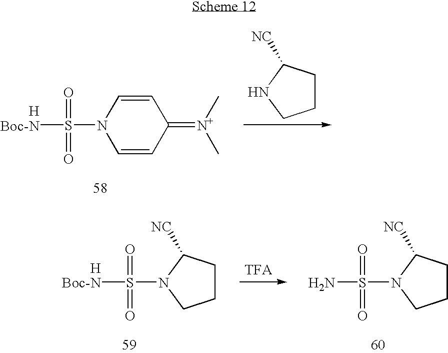

Step 1: Compound 58 was synthesized from chloro-sulfonyl-isothiocyanate as a white solid in 65% yield.

¹H NMR (DMSO-d₆, 400 MHz) δ 1.24 (s, 9H), 3.20 (s, 6H), 6.96 (d, J=8.34 Hz, 2H), 8.46 (d, J=8.34 Hz, 2H).

Step 2: Preparation of Boc-2-cyano-pyrrolidine-1-sulfonic acid amide 59. Compound 59 was synthesized from (S)-pyrrolidine-2-carbonitrile hydrochloride (0.754 mmol) and compound 58 as colorless oil in 77% yield.

¹H NMR (CDCl₃, 400 MHz): δ 1.45 (s, 9H), 2.05-2.25 (m, 4H), 3.32-3.38 (m, 2H), 4.98 (t, J=5.00 Hz, 1H), 7.99 (s, 1H).

Step 3: Preparation of 2-cyano-pyrrolidine-1-sulfonic acid amide 60. A solution of compound 59 (427 mg, 1 eq.) in a minimum amount of acetonitrile was poured onto a SCX-2 cartridge (biotage), which was heated at 55° C. for 6 hrs. The target compound was then eluted with NH₃/MeOH and concentrated under reduced pressure to yield compound 60 as a white solid in 99% yield.

¹H NMR (DMSO-d₆, 400 MHz) δ 1.21 (s, 2H), 1.87-1.94 (m, 2H), 2.09-2.24 (m, 2H), 3.17-3.26 (m, 2H), 4.52 (dd, J=4.64 and 7.88 Hz, 1H).

### Example 11

### Preparation of Cyclopropanesulfonic Acid 65

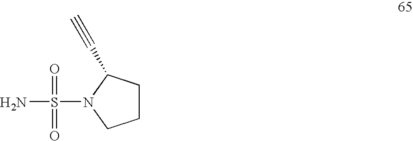

The synthesis of macrocyclic compound 65 is shown in Scheme 13.

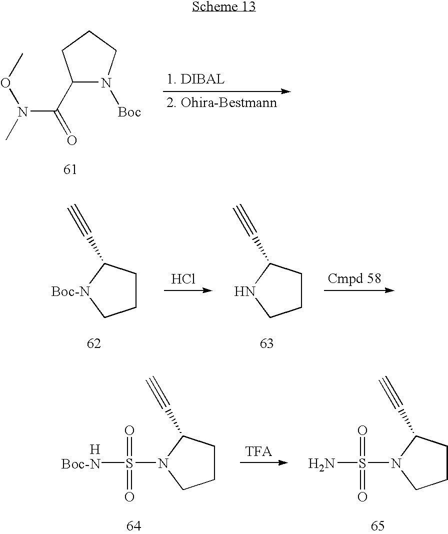

Step 1: Preparation of Boc-2-ethynyl-pyrrolidine 62. To a solution of 2-(methoxy-methyl-carbamoyl)-pyrrolidine-1-carboxylic acid-tert-butyl ester 61 (2 g, 1 eq.) in dry DCM (15 mL) was added 1 M DIBAL solution in heptane (9.3 mL, 1.2 eq.) at −78° C. under nitrogen over 15 min. After 1 hr, the mixture was quenched with MeOH (7 mL) and then allowed to warm up to 0° C. Bestmann-Ohira reagent (1.8 g, 1.2 eq.), K₂CO₃ (2.14 g, 2 eq.), and MeOH (7 mL) were added and the mixture was stirred at room temperature for 2 days. Rochelle's salt (1.2 eq.) in water was added and the mixture was vigorously stirred for 2 hrs. The mixture was then extracted with EtOAc. Organics were dried over Na₂SO₄ and concentrated under reduced pressure to yield compound 62 as colorless oil in 58% yield.

¹H NMR (CDCl₃, 400 MHz): δ 1.48 (s, 9H), 1.90-2.22 (m, 4H), 3.31-3.49 (m, 2H), 4.42-4.52 (m, 1H).

Step 2: Preparation of 2-ethynyl-pyrrolidine hydrochloride 63. To a solution of compound 62 (870 mg, 1 eq.) in diethyl ether was added 37% aqueous HCl (1.15 mL). The mixture was stirred at room temperature for 1.5 day, and then evaporated and sonicated in diethyl ether to yield compound 63 as a beige solid in quantitative yield.

¹H NMR (DMSO-d₆, 400 MHz) δ 2.02-2.22 (m, 4H), 3.40-3.49 (m, 2H), 4.32-4.42 (m, 1H).

Step 3: Preparation of Boc-2-ethynyl-pyrrolidine-1-sulfonic acid amide 64. Compound 64 was synthesized from compounds 58 (4.89 mmol) and 62 (4.45 mmol) as colorless oil in 94% yield, following the procedure described in Organic Letters 2001, 14, 2241-2243.

¹H NMR (DMSO-d₆, 400 MHz): δ 1.40 (s, 9H), 1.83-2.06 (m, 4H), 3.21-3.27 (m, 2H), 3.37-3.42 (m, 1H), 4.65-4.67 (m, 1H), 11.06 (s, 1H).

Step 4: Preparation of 2-ethynyl-pyrrolidine-1-sulfonic acid amide 65. Compound 65 was synthesized from compound 64 as a white solid in 99% yield, following the procedure described for compound 52a.

¹H NMR (DMSO-d₆, 400 MHz): δ 1.82-1.90 (m, 3H), 2.00-2.07 (m, 1H), 3.16-3.20 (m, 3H), 4.30 (d, J=7.7 Hz, 1H), 6.77 (s, 2H).

### Example 12

### Preparation of Cyclopropanesulfonic Acid 67

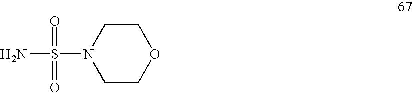

The synthesis of macrocyclic compound 67 is shown in Scheme 14.

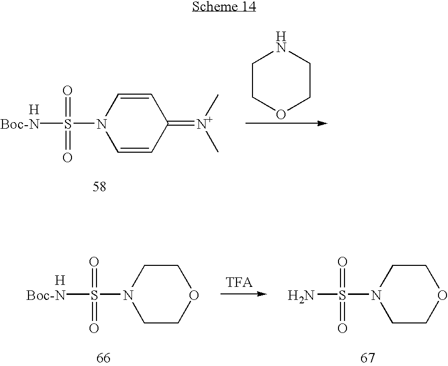

Step 1: Preparation of Boc-morpholine-4-sulfonic acid amide 66. Compound 66 was synthesized from compound 58 (3 g, 1 eq). and morpholine (0.87 mL, 1 eq.) as a white solid in 90% yield, following the procedure as described in WO 2006/007700.

¹H NMR (CDCl₃, 400 MHz) δ 1.50 (s, 9H), 3.40 (t, J=4.59 Hz, 4H), 3.76 (t, J=4.59 Hz, 4H), 7.02 (brs, 1H).

Step 2: Preparation of morpholine-4-sulfonic acid amide 67. Compound 67 was synthesized from compound 66 (2.4 g, 1 eq.) as a white solid in 82% yield, following the procedure as described for compound 52a.

¹H NMR (DMSO-d₆, 400 MHz) δ 2.90 (t, J=4.70 Hz, 4H), 3.63 (t, J=4.70 Hz, 4H), 6.81 (s, 2H).

### Example 13

### Preparation of 2-(4-isopropylthiazol-2-yl)-substituted quinolin-4-ols 76

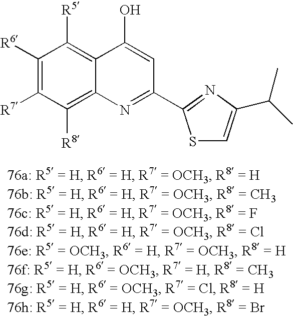

The syntheses of compounds 76 are shown in Schemes 15 to 17, where R^{5′}, R^{6′}, R^{7′}, and R^{8′} in compounds 72 to 80 are the same as defined in compounds 76.

### Method 1:

Step 1: Preparation of 1-bromo-3-methylbutan-2-one 68. To a solution of 3-methyl-2-butanone (40.7 g, 1 eq.) in ethanol (391 mL) was added bromide (62.4 g, 0.83 eq.) under nitrogen at 0° C. over 30 min. The reaction mixture was stirred at 0° C. for 4 hrs, then quenched with 1M aqueous sodium metabisulfite (100 mL) and extracted with petroleum ether (750 mL). The organic layer was washed twice with water (100 mL), twice with a cold saturated aqueous bicarbonate, and then brine. The organic layer was dried over sodium sulfate and then concentrated under reduced pressure. The product was purified by distillation under vacuum to yield compound 68 as colourless oil in 42% yield.

¹H NMR (CDCl₃, 400 MHz): δ 1.17 (d, J=6.98 Hz, 6H), 2.99 (m, J=6.98 Hz, 1H), 3.99 (s, 2H).

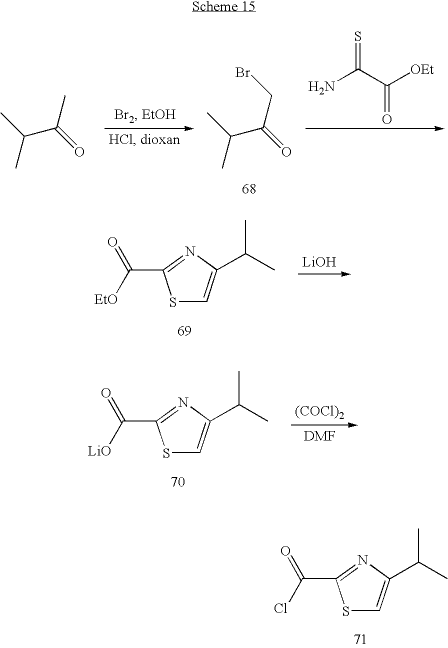

Step 2: Preparation of ethyl 4-isopropylthiazole-2-carboxylate 69. A solution of compound 68 (3.5 g, 1.25 eq.) and ethylthioxamate (2.3 g, 1 eq.) in ethanol (40 mL) was heated to 80° C. for 6 hrs, and then cooled to 0° C. The reaction mixture was diluted with water and EtOAc, and then neutralized to pH 7 with NH₃ (28%). The aqueous layer was extracted with EtOAc. The combined organic layers were dried over sodium sulfate and then removed under reduced pressure. The residue was purified by chromatography on silica gel to yield compound 69 as yellow oil in quantitative yield.

¹H NMR (DMSO-d₆, 400 MHz): δ 1.25 (d, J=6.73 Hz, 6H), 1.31 (t, J=7.24 Hz, 3H), 3.11 (hep, J=6.73 Hz, 1H), 4.35 (q, J=7.24 Hz, 2H), 7.72 (s, 1H).

Step 3: Preparation of 4-isopropylthiazole-2-carboxylic acid, lithium salt 70. To a solution of compound 69 (26 g, 1 eq.) in a mixture of MeOH (78 mL) and THF (260 mL), lithium hydroxide (2.8 g, 0.9 eq.) was added. The reaction mixture was stirred at room temperature overnight. The solvents were then removed under reduced pressure. The residue was triturated with petroleum ether (500 mL), filtrated, washed with petroleum ether, and dried under vacuum to yield compound 70 as a beige solid in 56% yield.

¹H NMR (DMSO-d₆, 400 MHz): δ 1.21 (d, J=6.73 Hz, 6H), 2.95 (hep, J=6.73 Hz, 1H), 7.19 (s, 1H).

Step 4: Preparation of 4-isopropylthiazole-2-carbonyl chloride 71. Oxalyl chloride (2.9 g, 1.5 eq.) was added dropwise under nitrogen at 0° C. to a suspension of compound 70 (1.8 g, 1 eq.) in DCM (25 mL) and DMF (50 μL). The reaction mixture was stirred at 0° C. for 30 min and then at room temperature for additional 90 min. Lithium chloride salt was removed from the reaction mixture through filtration. The solvent was then removed under reduced pressure to give compound 71 as yellow oil in quantitative yield, which was stored under nitrogen and used directly in the next step without further purification.

¹H NMR (DMSO-d6, 400 MHz): δ 1.21 (d, J=6.73 Hz, 6H), 2.95 (hep, J=6.73 Hz, 1H), 7.19 (s, 1H).

Step 5: Preparation of 1-(2-amino-4-methoxyphenyl)ethanone 73a. Trichloroborane (1M) in DCM (82 mL, 1 eq.) was added dropwise to a solution of meta-anisidine 72a (10 g, 1 eq.) in toluene (56 mL) under nitrogen at 0-5° C. over 1 hr. After stirred for 10 min at 0° C., ACN (5.2 mL, 1.20 eq.) was added. After the reaction mixture was stirred for additional 1 hr at 0° C., aluminium(III) chloride (11.9 g, 1.1 eq.) was added at 0° C. The reaction mixture was stirred at 50° C. for 16 hrs. The reaction mixture was then cooled down to 0° C., and propan-2-ol (38 mL) was added over 10 min, followed by addition of water (110 mL) over 30 min. The reaction mixture was heated to 50° C. for 3 hrs. After cooling down to 0° C., aqueous solution of sodium hydroxide (25%) was added. The aqueous layer was extracted with toluene (100 mL). The combined organic layers were washed with NaOH (25%), brine, and dried over sodium sulfate. The solvent was removed to yield compound 73a as a yellow solid in 63% yield.

¹H NMR (CDCl₃, 400 MHz): δ 2.52 (s, 3H), 3.80 (s, 3H), 6.07 (d, J=2.43, 1H), 6.23 (dd, J=2.43 and 8.98 Hz, 1H), 6.43 (br s, 2H), 7.63 (d, J=8.98 Hz).

Step 6: Preparation of 1-(2-amino-3-methyl-4-methoxyphenyl)ethanone 73b. Compound 73b was synthesized from 3-methoxy-2-methylaniline 72b as a yellow solid in 23% yield, according to the procedure as described for compound 73a.

MS (ESI, EI⁺): m/z=180 (MH⁺).

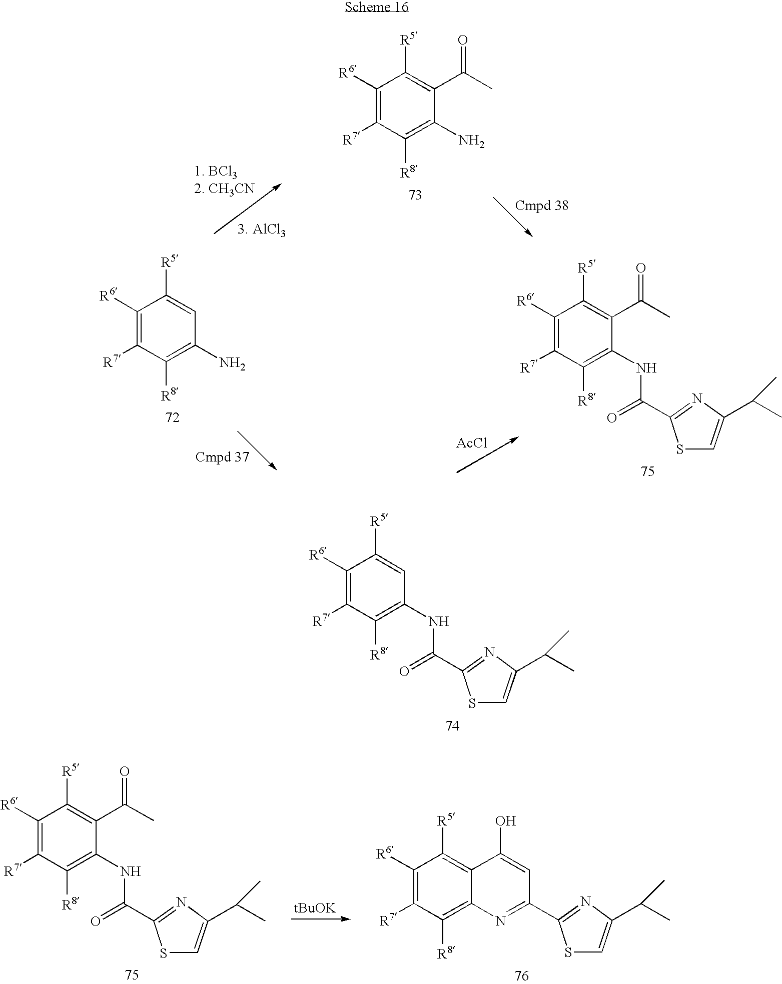

Step 7: Preparation of 1-(2-amino-4-chloro-5-methoxy-phenyl)-ethanone 73g. Compound 73g was synthesized from 3-chloro-4-methoxy-aniline 72g as a brown solid in 50% yield, according to the procedure as described for compound 73a.

MS (ESI, EI⁺): m/z=200 (MH⁺).

Step 8: Preparation of 1-(2-amino-3-bromo-4-methoxy-phenyl)-ethanone 73h. Compound 73h was synthesized from 2-bromo-3-methoxy-aniline 72h, following procedure described in WO 2007/014919.

Step 9: Preparation of N-(2-acetyl-5-methoxyphenyl)-4-isopropylthiazole-2-carboxamide 75a. Under nitrogen, a solution of compound 73a (3 g, 1 eq.) in 1,4-dioxane (30 mL) was added at 0° C. to a solution of compound 71 (4.1 g, 1.2 eq.) in 1,4-dioxane. The reaction mixture was stirred at room temperature overnight. The solvent was removed under reduced pressure and the residue was purified by chromatography on silica gel to yield compound as a beige solid 75a in 75% yield.

¹H NMR (CDCl₃, 400 MHz): δ (ppm) 1.43 (d, J=6.98 Hz, 6H), 2.65 (s, 3H), 3.26 (hep, J=6.98 Hz, 1H), 3.92 (s, 3H), 6.69 (dd, J=2.59 and 8.80 Hz, 1H), 7.2 (d, J=0.84, 1H), 7.87 (d, J=8.9 Hz, 1H), 8.58 (d, J=2.59 Hz, 1H), 13.5 (br s, 1H); MS (ESI, EI⁺): m/z=319 (MH⁺).

Step 10: Preparation of N-(6-acetyl-2-methyl-3-methoxyphenyl)-4-isopropylthiazole-2-carboxamide 75b. Compound 73b was synthesized from compound 73b and compound 71 as a beige solid in 66% yield, according to the procedure as described for compound 75a.

MS (ESI, EI⁺): m/z=333 (MH⁺).

Step 11: Preparation of N-(6-acetyl-2-fluoro-3-methoxyphenyl)-4-isopropylthiazole-2-carboxamide 75c. Compound 75c was synthesized from 1-(2-amino-3-fluoro-4-methoxyphenyl)ethanone 73c and compound 71 as a beige solid in 80% yield, according to the procedure as described for compound 75a.

MS (ESI, EI⁺): m/z=337 (MH⁺).

Step 12: Preparation of N-(6-acetyl-2-chloro-3-methoxyphenyl)-4-isopropylthiazole-2-carboxamide 75d. Compound 75d was synthesized from 1-(2-amino-3-chloro-4-methoxyphenyl)ethanone 73d and compound 71 as a beige solid in 80% yield, according to the procedure as described for compound 75a.

MS (ESI, EI⁺): m/z=353 (MH⁺).

Step 13: Preparation of N-(6-acetyl-3-chloro-4-methoxyphenyl)-4-isopropylthiazole-2-carboxamide 75g. Compound 75g was synthesized from compounds 70 and 73g as a beige solid in 69% yield, according to the procedure as described for compound 42a.

MS (ESI, EI⁺): m/z=354 (MH⁺).

Step 14: Preparation of N-(6-acetyl-2-bromo-3-methoxyphenyl)-4-isopropylthiazole-2-carboxamide 75h. Compound 75h was synthesized from compound 73h, following procedure described in WO 2007/014919.

Step 15: Preparation of N-(3,5-dimethoxy-phenyl)-4-isopropylthiazole-2-carboxamide 74e. To a stirred solution of compound 70 (1.38 g, 7.8 mmol) in DCM (50 mL) under nitrogen was added oxalyl chloride (1.16 g, 9.1 mmol). The reaction mixture was stirred at room temperature for 90 min. The solution was filtered under nitrogen and washed with DCM. The filtrate was concentrated under reduced pressure and the residue was dissolved in dioxane (20 mL). 3,5-Dimethoxyaniline (1 g, 6.5 mmol) in dioxane (9 mL) was added dropwise. The reaction mixture was stirred at room temperature for 90 min. Solvent was removed under reduced pressure and the crude material was purified by chromatography on silica gel (EtOAc/DCM) to yield compound 74e as a white solid in 90% yield.

¹H NMR (CDCl₃, 400 MHz) δ 1.35 (s, 3H), 1.37 (s, 3H), 3.14-3.17 (m, 1H), 3.82 (s, 6H), 6.30 (brs, 1H), 6.97 (d, J=2.30 Hz, 2H), 7.19 (s, 1H); MS (ESI, EI⁺) m/z=307 (MH⁺).

Step 16: Preparation of N-(2-acetyl-3,5-dimethoxy-phenyl)-4-isopropylthiazole-2-carboxamide 75e. To a suspension of Et₂AlCl (1.61 g, 12.04 mmol) in DCM at 0° C. was added acetyl chloride (630 mg, 8.02 mmol). The mixture was stirred at 0° C. for 30 min. Compound 74e (1.23 g, 4.01 mmol) was then added and the reaction mixture was stirred at 80° C. for 90 min. The reaction was poured in ice and DCM was added. The organic layers were separated, dried over Na₂SO₄, filtered, and concentrated under reduced pressure. The crude product was purified by chromatography on silica gel (EtOAc/DCM) to yield compound 74e as a white solid in 82% yield.

¹H NMR (CDCl₃, 400 MHz) δ 1.41 (s, 3H), 1.43 (s, 3H), 2.63 (s, 3H), 3.20-3.27 (m, 1H), 3.89 (s, 3H), 3.90 (s, 3H), 6.27 (d, J=2.30, 1H), 7.19 (s, 1H), 8.12 (d, J=2.30 Hz, 1H).

Step 17: Preparation of 2-(4-isopropylthiazol-2-yl)-7-methoxy-8-methylquinolin-4-ol 76b. Under nitrogen atmosphere, to a solution of compound 75b (4.3 g, 1 eq.) in THF (60 mL) was added potassium t-butoxide (3.8 g, 2.5 eq.). The mixture was heated to 70° C. for 16 hrs, and then cooled down to 0° C., quenched with methanol (10 mL) and acetic acid (2.5 mL). The solvent was removed under reduced pressure and the residue was triturated with a mixture of methanol/water. Solid was collected by filtration, and washed with acetonitrile and then petroleum ether to give compound 76b as a yellow solid in 60% yield.

MS (ESI, EI⁺): m/z=315 (MH⁺).

Step 18: Preparation of 2-(4-isopropylthiazol-2-yl)-7-methoxyquinolin-4-ol 76a. Compound 76a was synthesized from compound 76a as a yellow solid, according to the procedure as described for compound 76b.

¹H NMR (DMSO-d₆, 400 MHz): δ 1.32 (d, J=6.98 Hz, 6H), 3.14 (m, 1H), 3.89 (s, 3H), 7.06 (br s, 1H), 7.50-7.66 (m, 3H), 8 (d, J=9.05 Hz, 1H), 11.62 (br s, 1H); MS (ESI, EI⁺): m/z 301 (MH⁺).

Step 19: Preparation of 2-(4-isopropylthiazol-2-yl)-8-fluoro-7-methoxyquinolin-4-ol 76c. Compound 76c was synthesized from compound 75c as a yellow solid in 43% yield, according to the procedure as described for compound 76b.

MS (ESI, EI⁺): m/z=319 (MH⁺).

Step 20: Preparation of 2-(4-isopropylthiazol-2-yl)-8-chloro-7-methoxyquinolin-4-ol 76d. Compound 76d was synthesized from compound 75d as a yellow solid in 43% yield, according to the procedure as described for compound 76b.

MS (ESI, EI⁺): m/z=335 (MH⁺).

Step 21: Preparation of 2-(4-isopropylthiazol-2-yl)-5,7-dimethoxyquinolin-4-ol 76e. Compound 76e was synthesized from compound 75e as a yellow solid in 60% yield, according to the procedures as described for compound 76b.

¹H NMR (CDCl₃, 400 MHz) δ 1.37 (s, 3H), 1.39 (s, 3H), 3.15-3.22 (m, 1H), 3.95 (s, 3H), 4.05 (s, 3H), 6.45 (s, 1H), 7.03 (s, 2H), 7.62 (brs, 1H), 9.55 (s, 1H); MS (ESI, EI⁺): m/z 331 (MH⁺).

Step 22: Preparation of 7-chloro-2-(4-isopropylthiazol-2-yl)-6-methoxyquinolin-4-ol 76g. Compound 76g was synthesized from compound 75g as a yellow solid in 70% yield, according to the procedures as described for compound 76b.

MS (ESI, EI⁺): m/z=335 (MH⁺).

Step 23: Preparation of 8-bromo-7-methoxy-2-(4-isopropyl-thiazol-2-yl)-quinolin-4-ol 76h. Compound 76h was synthesized according to the procedures described in WO 2007/014919, the disclosure of which is incorporated herein by reference in its entirety.

MS (ESI, EI⁺): m/z=380 (MH⁺).

### Method B:

Step 1: Preparation of 4-isopropyl-2-tributylstannanyl-thiazole 77. To a stirred solution of 4-isopropylthiazole (9 g, 71 mmol) in anhydrous THF (100 mL) at −78° C. was added nBuLi (40 mL, 99 mmol). The reaction was stirred for 1 hr and the temperature reached −40° C. The reaction mixture was cooled to −78° C. and tri-n-butyltinchloride (23 g, 71 mmol) was added. The reaction mixture was stirred at room temperature for 48 hrs. Water was added and solvent was evaporated under reduced pressure. The residue was partioned between water and EtOAc. Organics were dried over Na₂SO₄, filtered, and concentrated under reduced pressure to yield compound 77 as colorless oil in 55% yield.

¹H NMR (CDCl₃, 400 MHz) δ 0.88-1.62 (m, 27H), 1.40 (s, 3H), 1.42 (s, 3H), 3.17-3.24 (m, 1H).

Step 2: Preparation of 2,4,8-trichloro-7-methoxyquinoline 78d. A mixture of 2-chloro-3-methoxyaniline hydrochloride 72d (15 g, 1 eq.), malonic acid (12.06 g, 1.5 eq.), and phosphorus oxochloride (80 mL) was refluxed for 16 hrs. The reaction mixture was slowly poured into water and extracted with DCM. The organic layer was dried over Na₂SO₄, filtered, and concentrated under reduced pressure. The crude material was purified on silica pad, eluted with DCM, to yield compound 78d as a white solid in 74% yield.

¹H NMR (CDCl₃, 376 MHz) δ 4.10 (s, 3H), 7.43 (t, J=4.88 Hz, 2H), 8.12 (d, J=9.48 Hz, 1H).

Step 3: Preparation of 2,4-dichloro-8-methyl-7-methoxyquinoline 78b. Compound 78b was synthesized from 2-methyl-3-methoxyaniline hydrochloride 72b and malonic acid as a white powder in 43% yield, following the procedure as described for compound 78d.

¹H NMR (CDCl₃, 376 MHz) δ 2.62 (s, 3H), 4.03 (s, 3H), 7.34 (s, 1H), 7.37 (d, J=9.02 Hz, 1H), 8.05 (d, J=9.02 Hz, 1H).

Step 4: Preparation of 2,4-dichloro-6-methoxy-8-methyl-quinoline 78f. A mixture of 4-methoxy-2-methyl aniline 72f (5 g, 36.45 mmol), malonic acid (5.68 g, 54.67 mmol) in phosphorus oxide trichloride (36 mL) was refluxed for 16 hrs. The reaction mixture was then poured dropwise into cooled water (400 mL), extracted with ethyl acetate, washed with brine, dried over Na₂SO₄, filtered, concentrated under reduced pressure, and purified by chromatography on silica gel (DCM) to yield compound 78f as a beige solid in 43% yield.

¹H NMR (CDCl₃, 400 MHz) δ 2.72 (s, 3H), 3.95 (s, 3H), 7.27-7.28 (m, 2H), 7.47 (s, 1H).

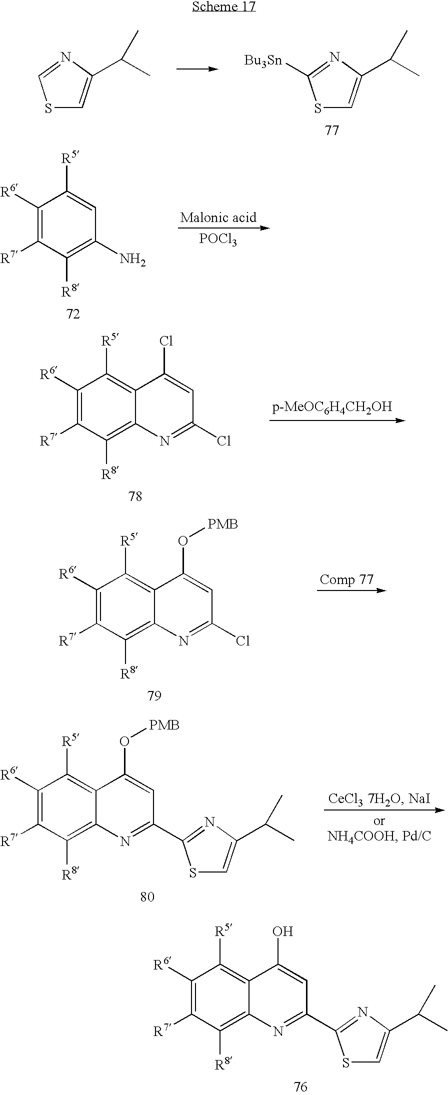

Step 5: Preparation of 2,8-dichloro-7-methoxy-4-(4-methoxy-benzyloxy)-quinoline 79d. NaH (60% in oil) (670 mg, 1.2 eq.) was added portionwise to a stirred solution of p-methoxybenzylalcohol (2.31 g, 1.2 eq.) and 15-crown-5 (3.32 mL, 1.2 eq.) in anhydrous DMF (10 mL). The mixture was stirred at room temperature for 30 min. Compound 78d (3.66 g, 1 eq.) in anhydrous DMF (25 mL) was then added and the reaction mixture was stirred at room temperature for 16 hrs. The reaction mixture was then poured into water (300 mL), extracted with EtOAC, dried over Na₂SO₄, filtered, and concentrated under reduced pressure. The crude material was purified by chromatography on silica gel (petroleum ether/DCM, 50/50) to give compound 79d as a yellow solid in 38% yield.

¹H NMR (CDCl₃, 376 MHz) δ 3.86 (s, 3H), 4.05 (s, 3H), 5.20 (s, 2H), 6.77 (s, 1H), 6.98 (d, J=8.53 Hz, 2H), 7.23 (d, J=9.41, 1H), 7.42 (d, J=8.53 Hz, 2H), 8.08 (d, J=9.41 Hz, 1H).

Step 6: Preparation of 2-chloro-8-methyl-7-methoxy-4-(4-methoxy-benzyloxy)-quinoline 79b. Compound 79b was synthesized from compound 78b as a white powder in 50% yield, following the procedure as described for compound 79d.

¹H NMR (CDCl₃, 376 MHz) δ 2.60 (s, 3H), 3.85 (s, 3H), 3.97 (s, 3H), 5.18 (s, 2H), 6.69 (s, 1H), 6.97 (d, J=8.57 Hz, 1H), 7.19 (d, J=8.57 Hz, 1H), 7.42 (d, J=8.57 Hz, 1H), 8.02 (d, J=8.57 Hz, 1H).

Step 7: Preparation of 2-chloro-6-methoxy-4-(4-methoxybenzyloxy)-8-methyl-quinoline 79f. Compound 79f was synthesized from compound 79f as a white solid in 58% yield, following the procedure as described for compound 79d. (58%).

¹H NMR (CDCl₃, 400 MHz) δ 2.68 (s, 3H), 3.80 (s, 3H), 3.83 (s, 3H), 5.11 (s, 2H), 6.72 (s, 1H), 6.97 (d, J=9.03 Hz, 2H), 7.15 (dd, J=3.01 Hz and J=0.96 Hz, 1H), 7.20 (d, J=3.00 Hz, 1H), 7.40 (d, J=9.03 Hz, 2H).

Step 8: Preparation of 2-(4-isopropyl-thiazol-2-yl)-6-methoxy-4-(4-methoxy-benzyloxy)-8-methyl-quinoline 80f. Compound 77 (100 mg, 0.29 mmol), compound 79f (242 mg, 0.35 mmol), and potassium carbonate (48 mg, 0.35 mmol) in degassed anhydrous DMF were stirred under microwave radiations at 80° C. for 1 hr. Solvent was removed under reduced pressure and the crude material was purified by chromatography on silica gel (Petroleum ether/DCM) to yield compound 80f as yellow powder in 63% yield.

¹H NMR (CDCl₃, 400 MHz) δ 1.40 (s, 3H), 1.42 (s, 3H), 2.80 (s, 3H), 3.17-3.24 (m, 1H), 3.85 (s, 3H), 3.89 (s, 3H), 5.31 (s, 2H), 6.99 (d, J=9.10 Hz, 2H), 7.00 (s, 1H), 7.21 (m, 1H), 7.31 (d, J=2.93 Hz, 1H), 7.49 (d, J=9.10 Hz, 2H), 7.79 (s, 1H).

Step 9: Preparation of 4-hydroxy-[2-(4-isopropyl-thiazol-2-yl)]-6-methoxy-8-methyl-quinoline 76f. Compound 80f (1.23 g, 2.82 mmol), cesium trichloride (1.58 g, 4.23 mmol), and sodium iodide (423 mg, 2.82 mmol) in ACN (26 mL) were stirred at 85° C. for 1 hr. The mixture was then filtered through celite and the solvent was evaporated. The brown solid obtained was suspended in water, pH was adjusted at 5 with 1N HCl. The mixture was extracted with DCM, dried over Na₂SO₄, filtered, concentrated under reduced pressure, and purified by chromatography on silica gel (petroleum ether/DCM) to yield compound 76f as a brown solid in 55% yield.

¹H NMR (CDCl₃, 400 MHz) δ 1.40 (d, J=6.91 Hz, 6H), 2.80 (s, 3H), 3.17-3.24 (m, 1H), 3.89 (s, 3H), 7.00 (s, 1H), 7.21 (m, 1H), 7.55 (s, 1H), 7.79 (s, 1H), 9.56 (brs, 1H).

### Example 14

### Preparation of 2-(pyrazol-4-yl)-quinolin-4-ol Derivatives 83

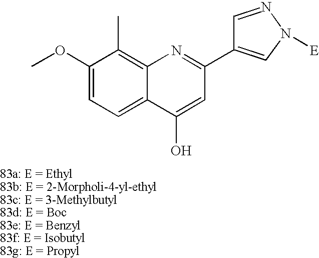

The syntheses of compounds 83 are shown in Scheme 18, where E in compound 82 is the same as defined in compound 83.

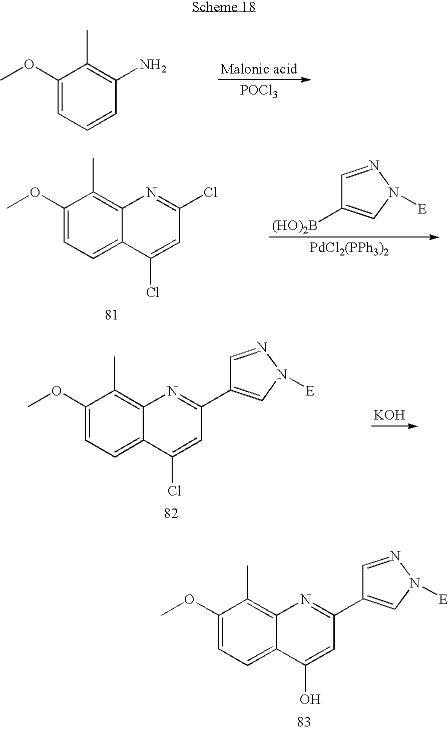

Step 1: Preparation of 2,4-dichloro-8-methyl-7-methoxyquinoline 81. A mixture of 2-methyl-3-methoxyaniline hydrochloride (15 g, 1 eq.), malonic acid (12.06 g, 1.5 eq.), and phosphorus oxochloride (80 mL) was refluxed for 16 hrs. The reaction mixture was slowly poured into water and extracted with DCM. The organic layer was dried over Na₂SO₄, filtered, and concentrated under reduced pressure. The crude material was purified on silica pad (eluted with DCM) to yield compound 81 as a white powder in 43% yield.

¹H NMR (CDCl₃, 376 MHz) δ 2.62 (s, 3H), 4.03 (s, 3H), 7.34 (s, 1H), 7.37 (d, J=9.02 Hz, 1H), 8.05 (d, J=9.02 Hz, 1H).

Step 2: Preparation of 4-chloro-2-(1-ethyl-pyrazol-4-yl)-7-methoxy-8-methyl-quinoline 82a. A solution of compound 81 (1 g, 1 eq.) and 1-ethyl-pyrazole-4-boronic acid pinacol ester (0.9 g, 1 eq.) in anhydrous DMF (30 mL) was heated at 95° C. Potassium carbonate (0.5 g, 0.8 eq.) and bis(triphenylphosphine)palladium(II)chloride (0.58 g, 0.2 eq.) were added. The reaction mixture was stirred at 95° C. for 16 hrs. The reaction mixture was then filtered through celite and partitioned between EtOAc and water. Organic phase was washed twice with brine, dried over Na₂SO₄, filtered, and concentrated under reduced pressure. The crude material was purified by chromatography on silica gel to give compound 82a as a white solid in 63% yield.

MS (ESI, EI⁺) m/z=302 (MH⁺).

Step 3: Preparation of 4-chloro-7-methoxy-8-methyl-2-[1-(2-morpholin-4-yl-ethyl)-pyrazol-4-yl]-quinoline 82b. Compound 82b was synthesized from compound 81 and 1-(2-morpholinoethyl)-1H-pyrazole-4-boronic acid as an off-white solid in 67% yield, following the procedure as described for compound 82a.

MS (ESI, EI⁺): m/z=387 (MH⁺).

Step 4: Preparation of 4-chloro-7-methoxy-8-methyl-2-[1-(3-methylbutyl)-pyrazol-4-yl]-quinoline 82c. Compound 82c was synthesized from compound 81 and 1-(3-methylbutyl)-1H-pyrazole-4-boronic acid pinacol ester as a white solid in 69% yield, following the procedure as described for compound 82a.

MS (ESI, EI⁺): m/z=344 (MH⁺).

Step 5: Preparation of 4-(4-chloro-7-methoxy-8-methyl-quinolin-2-yl)-pyrazole-1-carboxylic acid tert-butyl ester 82d. Compound 82d was synthesized from compound 81 and 1-carboxylic acid tert-butyl ester-1H-pyrazole-4-boronic acid pinacol ester as a white solid in 50% yield, following the procedure as described for compound 82a.

MS (ESI, EI⁺): m/z=374 (MH⁺).

Step 6: Preparation of 2-(1-benzyl-pyrazol-4-yl)-4-chloro-7-methoxy-8-methyl-quinoline 82e. Compound 82e was synthesized from compound 81 and 1-benzyl-1H-pyrazole-4-boronic acid pinacol ester as a white solid in 57% yield, following the procedure as described for compound 82a.

MS (ESI, EI⁺): m/z=374 (MH⁺).

Step 7: Preparation of 4-chloro-2-(1-isobutyl-pyrazol-4-yl)-7-methoxy-8-methyl-quinoline 82f. Compound 82f was synthesized from compound 81 and 1-isobutyl-1H-pyrazole-4-boronic acid pinacol ester as a white solid in 56% yield, following the procedure as described for compound 82a.

MS (ESI, EI⁺): m/z=330 (MH⁺).

Step 8: Preparation of 4-chloro-7-methoxy-8-methyl-2-(1-propyl-pyrazol-4-yl)-quinoline 82g. Compound 82f was synthesized from compound 81 and 1-propyl-1H-pyrazole-4-boronic acid pinacol ester as a white solid in 75% yield, following the procedure as described for compound 82a.

MS (ESI, EI⁺): m/z=316 (MH⁺).

Step 9: Preparation of 2-(1-ethyl-pyrazol-4-yl)-4-hydroxy-7-methoxy-8-methyl-quinoline 83a. A mixture of compound 82a (100 mg) and KOH (190 mg) in DMSO (1 mL) was stirred at 100° C. for 2 days. The mixture was then partitioned between EtOAc and water. Organic phase was washed with brine, dried over Na₂SO₄, filtered, and concentrated under reduced pressure. The crude material was purified by chromatography on silica gel to give compound 83a as a white solid in 36% yield.

MS (ESI, EI⁺): m/z=284 (MH⁺).

Step 10: Preparation of 4-hydroxy-7-methoxy-8-methyl-2-[1-(2-morpholin-4-yl-ethyl)-pyrazol-4-yl]-quinoline 83b. Compound 83b was synthesized from compound 82b as an off-white solid in 58% yield, following the procedure as described for compound 83a.

MS (ESI, EI⁺): m/z=369 (MH⁺).

Step 11: Preparation of 4-hydroxy-7-methoxy-8-methyl-2-[1-(3-methylbutyl)-pyrazol-4-yl]-quinoline 83c. Compound 83c was synthesized from compound 82c as a white solid in 66% yield, following the procedure as described for compound 83a.

MS (ESI, EI⁺): m/z=326 (MH⁺).

Step 12: Preparation of 4-(4-chloro-7-methoxy-8-methyl-quinolin-2-yl)-pyrazole-1-carboxylic acid tert-butyl ester 83d. Compound 83d was synthesized from compound 82d as a white solid in 40% yield, following the procedure as described for compound 83a.

MS (ESI, EI⁺): m/z=356 (MH⁺).

Step 13: Preparation of 2-(1-benzyl-pyrazol-4-yl)-4-hydroxy-7-methoxy-8-methyl-quinoline 83e. Compound 83e was synthesized from compound 82e as a white solid in 36% yield, following the procedure as described for compound 83a.

MS (ESI, EI⁺): m/z=346 (MH⁺).

Step 14: Preparation of 4-hydroxy-2-(1-isobutyl-pyrazol-4-yl)-7-methoxy-8-methyl-quinoline 83f. Compound 83f was synthesized from compound 82f as a white solid in 55% yield, following the procedure as described for compound 83a.

MS (ESI, EI⁺): m/z=312 (MH⁺).

Step 15: Preparation of 4-hydroxy-7-methoxy-8-methyl-2-(1-propyl-pyrazol-4-yl)-quinoline 83g. Compound 83g was synthesized from compound 82g as a white solid in 92% yield, following the procedure as described for compound 83a.

MS (ESI, EI⁺): m/z=298 (MH⁺).

### Example 15

### Preparation of a 2-(pyrazol-5-yl)-quinolin-4-ol Derivative 85

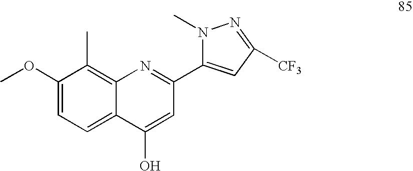

The syntheses of compounds 83 are shown in Scheme 19.

Step 1: Preparation of 4-chloro-2-(1-methyl-3-trifluoro-pyrazol-5-yl)-7-methoxy-8-methyl-quinoline 84. Compound 84 was synthesized from compound 81 (2.4 g, 1 eq.) and 1-methyl-3-trifluoro-methyl-pyrazol-5-boronic acid (2 g, 1 eq.) as a white solid in 76% yield, following the procedure as described for compound 82a.

MS (ESI, EI⁺): m/z=356 (MH⁺).

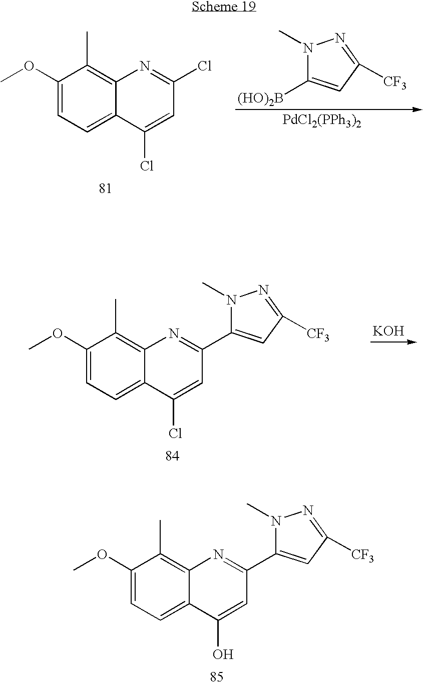

Step 2: Preparation of 4-hydroxy-2-(1-methyl-3-trifluoro-pyrazol-5-yl)-7-methoxy-8-methyl-quinoline 85. Compound 85 was synthesized from compound 84 (2.8 g, 1 eq.) as a white solid in 38% yield, following the procedure as described for compound 83a.

MS (ESI, EI⁺): m/z=338 (MH⁺).

### Example 16

### Preparation of Substituted Quinolines 91

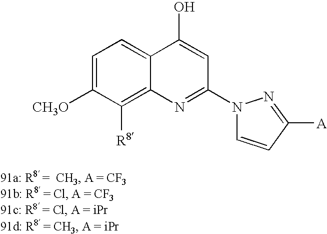

The syntheses of substituted quinolines are illustrated in Scheme 20, where R^{8′} and A in compound 90 are the same as defined in compound 91.

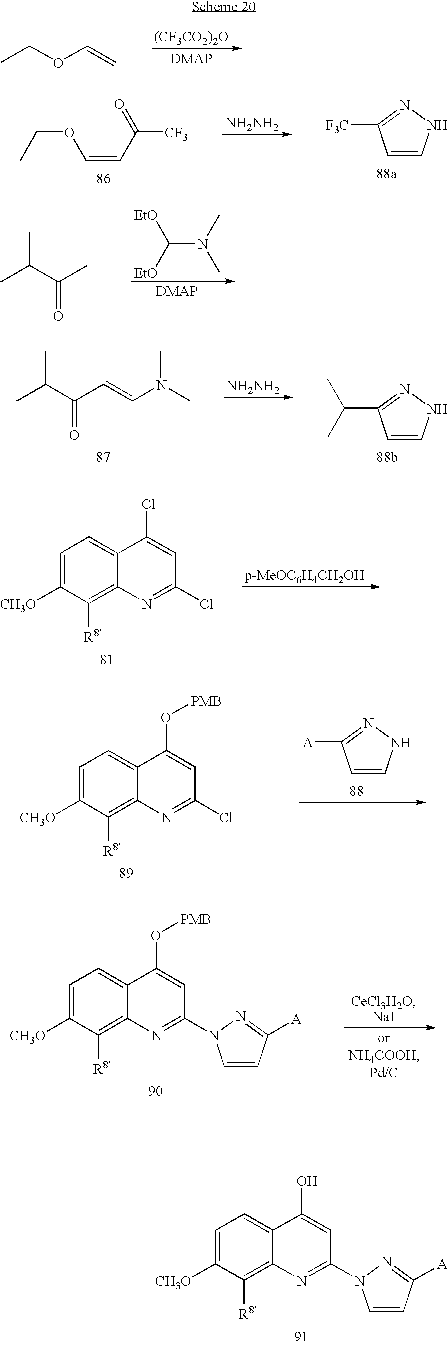

Step 1: Preparation of 4-ethoxy-trifluoro-but-3-en-2-one 86. Ethylvinylether (5 g, 1 eq.) was added dropwise at −10° C. and under nitrogen to a stirred solution of trifluoroacetic anhydride (10 mL, 1.05 eq.) and 4-dimethylaminopyridine (80 mg, 0.06 eq.) in DCM (90 mL). The reaction mixture was stirred at 0° C. for 8 hrs and allowed to warm up at room temperature overnight. The mixture was then poured into cold aqueous NaHCO₃ solution. The organic layer was separated, washed with water and brine, dried over Na₂SO₄, filtered, and concentrated under reduced pressure to yield compound 86 as brown oil in 87% yield.

¹H NMR (CDCl₃, 400 MHz) δ 1.39-1.43 (t, J=7.04 Hz, 3H), 4.08-4.13 (q, J=7.04 Hz, 2H), 5.86 (d, J=12.40 Hz, 1H), 7.90 (d, J=12.40 Hz, 1H).

Step 2: Preparation of 3-trifluoromethyl-1H-pyrazole 88a. To a stirred solution of hydrazine monochloride (6.62 g, 1.6 eq.) in EtOH (300 mL) was added dropwise compound 86 (10.16 g, 1 eq.) in EtOH (200 mL). The reaction mixture was refluxed for 6 hrs and evaporated to dryness. Water and EtOAc were added to the residue. The organic layer was washed with water and brine, dried over Na₂SO₄, filtered, and concentrated under reduced pressure to yield compound 88a as a brown solid in 86% yield.

¹H NMR (CDCl₃, 376 MHz) δ 6.66 (d, J=2.30 Hz, 1H), 7.72 (d, J=2.30 Hz, 1H);

¹⁹F NMR (CDCl₃, MHz) δ 61.41 (s, 3F).

Step 3: Preparation of 1-dimethylamino-4-methyl-pent-1-en-3-one 87. 3-Methylbutan-2-one (2.5 g, 1 eq.) and dimethylformamide diethylacetal (7.46 mL, 1.5 eq.) were stirred at 100° C. for 4 days in a sealed tube. The reaction mixture was used directly in the next step without further purification.

Step 4: Preparation of 3-isopropyl-1H-pyrazole 88b. Compound 87 (6.6 g, 1 eq.) was added dropwise to a stirred solution of hydrazine monochloride (3.2 g, 1 eq.), sulfuric acid (1.13 mL) and H₂O (6 mL). The reaction mixture was stirred at 68° C. for 2 hrs. The mixture was then neutralized with 1N NaOH and extracted with diethyl ether. The organic layer was dried over Na₂SO₄, filtered, and concentrated under reduced pressure to yield compound 88b as a beige solid in 94% yield.

¹H NMR (DMSO-d₆, 400 MHz) δ 1.17 (s, 3H), 1.19 (s, 3H), 2.87-2.93 (m, 1H), 5.99 (s, 1H), 7.40 (s, 1H). 1.39-1.43 (t, J=7.04 Hz, 3H), 4.08-4.13 (q, J=7.04 Hz, 2H), 5.86 (d, J=12.40 Hz, 1H), 7.90 (d, J=12.40 Hz, 1H).

Step 5: Preparation of 2-chloro-8-methyl-7-methoxy-4-(4-methoxy-benzyloxy)-quinoline 89a. NaH (60% in oil, 670 mg, 1.2 eq.) was added portionwise to a stirred solution of p-methoxybenzylalcohol (2.31 g, 1.2 eq.) and 15-crown-5 (3.32 mL, 1.2 eq.) in anhydrous DMF (10 mL). The mixture was stirred at room temperature for 30 min. Compound 81 (3.66 g, 1 eq.) in anhydrous DMF (25 mL) was then added and the reaction mixture was stirred at room temperature for 16 hrs. The reaction mixture was then poured into water (300 mL). The reaction mixture was extracted with EtOAC, dried over Na₂SO₄, filtered, and concentrated under reduced pressure. The crude material was purified by chromatography on silica gel (petroleum ether/DCM, 50/50) to give compound 89a as a white solid in 50% yield.

¹H NMR (CDCl₃, 376 MHz) δ 2.60 (s, 3H), 3.85 (s, 3H), 3.97 (s, 3H), 5.18 (s, 2H), 6.69 (s, 1H), 6.97 (d, J=8.57 Hz, 1H), 7.19 (d, J=8.57 Hz, 1H), 7.42 (d, J=8.57 Hz, 1H), 8.02 (d, J=8.57 Hz, 1H).

Step 6: Preparation of 2,8-dichloro-7-methoxy-4-(4-methoxy-benzyloxy)-quinoline 89b. Compound 89b was synthesized from 2,4,8-trichloro-7-methoxyquinoline as a yellow solid in 38% yield, following the procedure as described for compound 89a.

¹H NMR (CDCl₃, 376 MHz) δ 3.86 (s, 3H), 4.05 (s, 3H), 5.20 (s, 2H), 6.77 (s, 1H), 6.98 (d, J=8.53 hz, 2H), 7.23 (d, J=9.41, 1H), 7.42 (d, J=8.53 Hz, 2H), 8.08 (d, J=9.41 Hz, 1H).

Step 7: Preparation of 7-methoxy-8-methyl-4-(4-methoxy-benzyloxy)-2-(3-trifluoromethyl-pyrazol-1-yl)-quinoline 90a. To a stirred solution of compound 88a (821 mg, 1.1 eq.) in anhydrous DMF (20 mL) was added NaH (241 mg, 1.1 eq.) portionwise at 0° C. After the reaction mixture was stirred for 1 hr at room temperature, compound 89a (2 g, 1 eq) was added and the mixture was stirred at 90° C. for 16 hrs. EtOAc was added. The organic phase was washed with HCl (2.5 N), dried over Na₂SO₄, filtered, and concentrated under reduced pressure. The crude material was purified by chromatography on silica gel (petroleum ether/DCM, 50/50) to give compound 90a as a white solid in 19% yield.

¹H NMR (CDCl₃, 400 MHz) δ 2.64 (s, 3H), 3.86 (s, 3H), 3.99 (s, 3H), 5.33 (s, 2H), 6.75 (d, J=2.58 Hz, 1H), 6.98 (d, J=8.78 Hz, 2H), 7.20 (d, J=9.22 Hz, 1H), 7.48 (d, J=8.78 Hz, 2H), 7.57 (s, 1H), 8.07 (d, J=9.08 Hz, 1H), 8.88 (s, 1H).

Step 8: Preparation of 8-chloro-7-methoxy-4-(4-methoxy-benzyloxy)-2-(3-trifluoromethyl-pyrazol-1-yl)-quinoline 90b. Compound 90b was synthesized from compounds 88a and 89b as a white solid in 51% yield, following the procedure as described for compound 90a.

MS (ESI, EI⁻) m/z=461.9 (MH⁻).

Step 9: Preparation of 4-hydroxy-7-methoxy-8-methyl-2-(3-trifluoromethyl-pyrazol-1-yl)-quinoline 91a. A mixture of compound 90a (885 mg, 1.99 mmol), ammonium formate (629 mg, 9.98 mmol), and Pd/C (89 mg, 10% w) in EtOH (16 mL) was refluxed for 1 hr. The reaction was then filtered though celite and concentrated under reduced pressure. The residue was diluted with DCM and washed with water. Organics were dried over Na₂SO₄, filtered, concentrated under reduced pressure, and purified by chromatography on silica gel (petroleum ether/EtOAc) to yield compound 91a as a white solid in 93%.

¹H NMR (DMSO-d₆, 400 MHz) δ 2.54 (s, 3H), 3.94 (s, 3H), 7.06 (d, J=2.48 Hz, 1H), 7.37-7.40 (m, 2H), 8.02 (d, J=9.18 Hz, 1H), 8.97 (s, 1H), 11.89 (s, 1H).

Step 10: Preparation of 8-chloro-7-methoxy-4-hydroxy-2-(3-trifluoromethyl-pyrazol-1-yl)-quinoline 91b. Compound 90b (800 mg, 1 eq.), CeCl₃.7H₂O (965 mg, 1.5 eq.), and NaI (258 mg, 1 eq.) in ACN (10 mL) were stirred at 85° C. for 1 hr under microwave irradiations. Water was added and the mixture was acidified with 1N HCl to pH 5. The reaction mixture was extracted with diethyl ether. The organic layer was dried over Na₂SO₄, filtered, and concentrated under reduced pressure. The crude material was purified by chromatography on silica gel (MeOH/DCM) to give compound 91b as a beige solid in 96% yield.

¹H NMR (DMSO-d₆, 400 MHz) δ 4.02 (s, 3H), 7.07 (s, 1H), 7.43 (s, 1H), 7.51 (d, J=9.11 Hz, 1H), 8.11 (d, J=9.11 Hz, 1H), 8.88 (s, 1H); MS (ESI, EI⁺): m/z=343.9 (MH⁺).

Step 11: Preparation of 8-chloro-4-hydroxy-7-methoxy-2-(3-isopropyl-pyrazol-1-yl)-quinoline 91c. A mixture of compounds 88b (452 mg, 4.11 mmol) and 89b (500 mg, 1.37 mmol) in N-methylpyrrolidone (2 mL) was stirred at 200° C. for 30 min under microwave irradiations. Water was then added. The reaction mixture was extracted with EtOAc, dried over Na₂SO₄, filtered, concentrated under reduced pressure, and purified by chromatography on silica gel (DCM/EtOAc) to yield compound 91c as a white solid in 35% yield.

¹H NMR (DMSO-d₆, 400 MHz) δ 1.26 (s, 3H), 1.28 (s, 3H), 2.98-3.01 (m, 1H), 4.00 (s, 3H), 6.46 (m, 1H), 7.16 (d, 9.32 Hz, 1H), 7.89 (d, J=9.32 Hz, 1H), 8.05 (d, J=10.85 Hz, 1H), 8.60 (m, 1H), 10.69 (s, 1H).

### Example 17

### Preparation of Macrocyclic 94

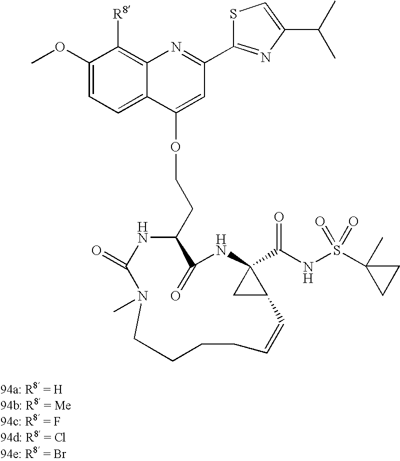

The syntheses of macrocyclic compounds are illustrated in Scheme 21, where R^{8′} and A in compounds 92 and 93 are the same as defined in compound 94.

Step 1: Preparation of (1R,4S,14S,Z)-ethyl 4-(2-(2-(4-isopropylthiazol-2-yl)-7-methoxy-8-methylquinolin-4-yloxy)ethyl)-7-methyl-3,6-dioxo-2,5,7-triazabicyclo[12.1.0]pentadec-12-ene-1-carboxylate 92b. To a solution of compounds 48 (500 mg, 1 eq.) and 76b (430 mg, 1 eq.) and triphenylphosphine (713 mg, 2 eq.) in THF (15 mL) was added dropwise DIAD (5.359 mL, 2 eq.) under nitrogen at 0° C. The reaction mixture was stirred at room temperature overnight. The solvent was evaporated and the crude residue was dissolved in EtOAc. The organic layer was washed with a NaHCO₃ saturated solution, followed by brine, and then dried on sodium sulphate. The solvent was removed under reduced pressure and the residue was purified by chromatography on silica gel to give compound 92b as pale yellow solid in 26% yield.

MS (ESI, EI⁺): m/z=664 (MH⁺).

Step 2: Preparation of (1R,4S,14S,Z)-ethyl 4-(2-(2-(4-isopropylthiazol-2-yl)-7-methoxy-quinolin-4-yloxy)ethyl)-7-methyl-3,6-dioxo-2,5,7-triazabicyclo[12.1.0]pentadec-12-ene-1-carboxylate 92a. Compound 92a was synthesized from compounds 48 and 76a in 30% yield, following the procedure as described for compound 92b (30%).

MS (ESI, EI⁻): m/z=648 (MH⁻).

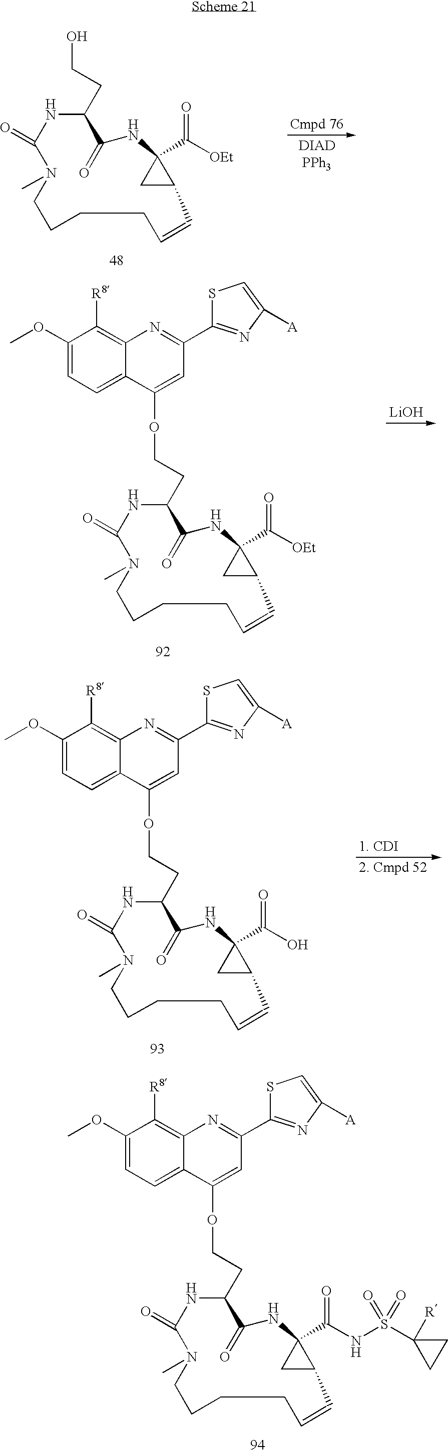

Step 3: Preparation of (1R,4S,14S,Z)-ethyl 4-(2-(2-(4-isopropylthiazol-2-yl)-7-methoxy-8-fluoro-quinolin-4-yloxy)ethyl)-7-methyl-3,6-dioxo-2,5,7-triazabicyclo[12.1.0]pentadec-12-ene-1-carboxylate 92c. Compound 92c was synthesized from compounds 48 and 76c as a beige solid in 29% yield, following the procedure as described for compound 92b.

MS (ESI, EI⁻): m/z=666 (MH⁻).

Step 4: Preparation of (1R,4S,14S,Z)-ethyl 4-(2-(2-(4-isopropylthiazol-2-yl)-7-methoxy-8-chloro-quinolin-4-yloxy)ethyl)-7-methyl-3,6-dioxo-2,5,7-triazabicyclo[12.1.0]pentadec-12-ene-1-carboxylate 92d. Compound 92d was synthesized from compounds 48 and 76d as a beige solid in 35% yield, following the procedure as described for compound 92b.

MS (ESI, EI⁺): m/z=682 (MH⁺).

Step 5: Preparation of (1R,4S,14S,Z)-ethyl 4-(2-(2-(4-isopropylthiazol-2-yl)-7-methoxy-8-bromo-quinolin-4-yloxy)ethyl)-7-methyl-3,6-dioxo-2,5,7-triazabicyclo[12.1.0]pentadec-12-ene-1-carboxylate 92e. Compound 92e was synthesized from compounds 48 and 76h as a white solid in 63% yield, following the procedure as described for compound 92b.

MS (ESI, EI⁺): m/z=729 (MH⁺).

Step 6: Preparation of (1R,4S,14S,Z)-4-(2-(2-(4-isopropylthiazol-2-yl)-7-methoxy-8-methylquinolin-4-yloxy)ethyl)-7-methyl-3,6-dioxo-2,5,7-triazabicyclo[12.1.0]pentadec-12-ene-1-carboxylic acid 93b. To a stirred solution of compound 92b (240 mg, 1 eq.) in THF (16 mL) was added aqueous LiOH (112 mg, 10 eq.) was added. The reaction mixture was stirred at 45° C. for 16 hrs. The solution was diluted with water, adjusted at pH 5 with 1N HCl, and extracted with ethyl acetate. The aqueous layer was treated with solid NaCl and then extracted again with ethyl acetate. Dried organics were concentrated under reduced pressure. The crude material was purified by chromatography on silica gel to give compound 93b as a pale yellow solid in 40% yield.

MS (ESI, EI⁺): m/z=636 (MH⁻).

Step 7: Preparation of (1R,4S,14S,Z)-4-(2-(2-(4-isopropylthiazol-2-yl)-7-methoxy-quinolin-4-yloxy)ethyl)-7-methyl-3,6-dioxo-2,5,7-triazabicyclo[12.1.0]pentadec-12-ene-1-carboxylic acid 93a. Compound 93a was synthesized from compound 92a as a white solid in 73% yield, following the procedure as described for compound 93b.

MS (ESI, EI⁻): m/z=648 (MH⁻).

Step 8: Preparation of (1R,4S,14S,Z)-4-(2-(2-(4-isopropylthiazol-2-yl)-7-methoxy-8-fluoro-quinolin-4-yloxy)ethyl)-7-methyl-3,6-dioxo-2,5,7-triazabicyclo[12.1.0]pentadec-12-ene-1-carboxylic acid 93c. Compound 93c was synthesized from compound 92c as a yellow solid in quantitative yield, following the procedure as described for compound 93b.

MS (ESI, EI⁺): m/z=640 (MH⁺).

Step 9: Preparation of (1R,4S,14S,Z)-4-(2-(2-(4-isopropylthiazol-2-yl)-7-methoxy-8-chloro-quinolin-4-yloxy)ethyl)-7-methyl-3,6-dioxo-2,5,7-triazabicyclo[12.1.0]pentadec-12-ene-1-carboxylic acid 93d. Compound 93d was synthesized from compound 92d as a beige solid in 45% yield, following the procedure as described for compound 93b.

MS (ESI, EI⁺): m/z=656 (MH⁺).

Step 10: Preparation of (1R,4S,14S,Z)-4-(2-(2-(4-isopropylthiazol-2-yl)-7-methoxy-8-bromo-quinolin-4-yloxy)ethyl)-7-methyl-3,6-dioxo-2,5,7-triazabicyclo[12.1.0]pentadec-12-ene-1-carboxylic acid 93e. Compound 93e was synthesized from compound 92e as a yellow solid in 43% yield, following the procedure as described for compound 93b.

¹H NMR (CDCl₃, 400 MHz) δ 1.36-1.39 (dd, J=3.50 and 3.20 Hz, 6H), 1.54-1.70 (m, 8H), 2.79 (s, 3H), 3.26-3.32 (m, 1H), 4.08 (s, 3H), 4.95-4.98 (m, 1H), 5.17-5.22 (t, J=10.27 Hz, 1H), 5.65-5.72 (m, 1H), 7.09 (s, 1H), 7.25 (d, J=8.8 Hz, 1H), 7.67 (brs, 1H), 8.13 (d, J=8.8 Hz, 1H); MS (ESI, EI⁺): m/z=701 (MH⁺).

Step 11: Preparation of (1R,4S,14S,Z)-4-(2-(2-(4-isopropylthiazol-2-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 94b. A mixture of compound 93b (60 mg, 1 eq.) and CDI (30 mg, 4 eq.) in THF (5 mL) was stirred under microwaves irradiations for 2 hrs at 90° C. 1-Methylcyclopropylsulfonylamide (25 mg, 2 eq.) and DBU (28 mg, 2 eq) were added and the mixture was stirred under microwaves irradiations for 1 hr at 80° C. After evaporation, the crude material was purified by chromatography on silica gel to give compound 94b as a pale yellow solid in 24% yield.

MS (ESI, EI⁺): m/z=753 (MH⁻).

Step 12: Preparation of a sodium salt of compound 94b. Compound 94b (6 mg) was dissolved in EtOAc and 2 drops of MeOH. Sodium methoxide (1 mg) was then added and the reaction mixture was stirred at 40° C. for 1 hr. Water was added, a red precipitate was formed, filtered, and dried over P₂O₅ under reduced pressure to yield a pale brown powder.

MS (ESI, EI⁺) m/z=775 (M+Na).

Step 13: Preparation of (1R,4S,14S,Z)-4-(2-(2-(4-isopropylthiazol-2-yl)-7-methoxy-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 94a. Compound 94a was synthesized from compound 93a as a beige solid in 25% yield, following the procedure as described for compound 94b.

MS (ESI, EI⁺): m/z=754 (MH⁺).

Step 14: Preparation of (1R,4S,14S,Z)-4-(2-(2-(4-isopropylthiazol-2-yl)-7-methoxy-8-fluoro-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 94c. Compound 94c was synthesized from compound 93c as a white solid in 11% yield, following the procedure as described for compound 94b.

MS (ESI, EI⁺): m/z=757 (MH⁺).

Step 15: Preparation of (1R,4S,14S,Z)-4-(2-(2-(4-isopropylthiazol-2-yl)-7-methoxy-8-chloro-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 94d. Compound 94d was synthesized from compound 93d as a white solid in 19% yield, following the procedure as described for compound 94b.

MS (ESI, EI⁺): m/z=787 (MH⁺).

Step 16: Preparation of (1R,4S,14S,Z)-4-(2-(2-(4-isopropylthiazol-2-yl)-7-methoxy-8-bromo-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 94e. Compound 94e was synthesized from compound 93e as a white solid in 33% yield, following the procedure as described for compound 94b.

MS (ESI, EI⁺): m/z=818 (MH⁺).

### Example 18

### Preparation of Macrocyclic Compound 95

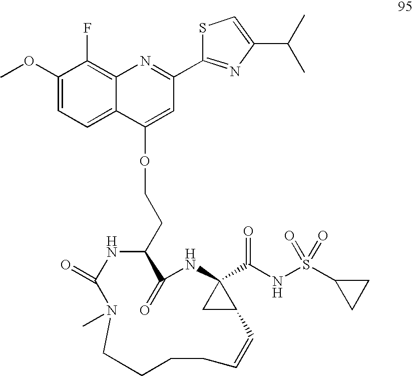

Preparation of (1R,4S,14S,Z)-4-(2-(2-(4-isopropylthiazol-2-yl)-7-methoxy-8-fluoro-quinolin-4-yloxy)ethyl)-7-methyl-N-cyclopropylsulfonyl-3,6-dioxo-2,5,7-triazabicyclo[12.1.0]pentadec-12-ene-1-carboxamide 95. Compound 95 was synthesized from compound 93c (62 mg, 1 eq.) and cyclopropanesulfonyl amide (23 mg, 2 eq.), as a white solid in 10% yield, following the procedure as described for compound 94b.

MS (ESI, EI⁺): m/z=743 (MH⁺).

### Example 19

### Preparation of Macrocyclic Compounds 96

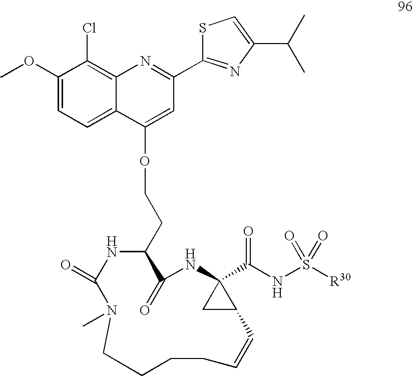

The syntheses of macrocyclic compounds 96 are illustrated in Scheme 22.

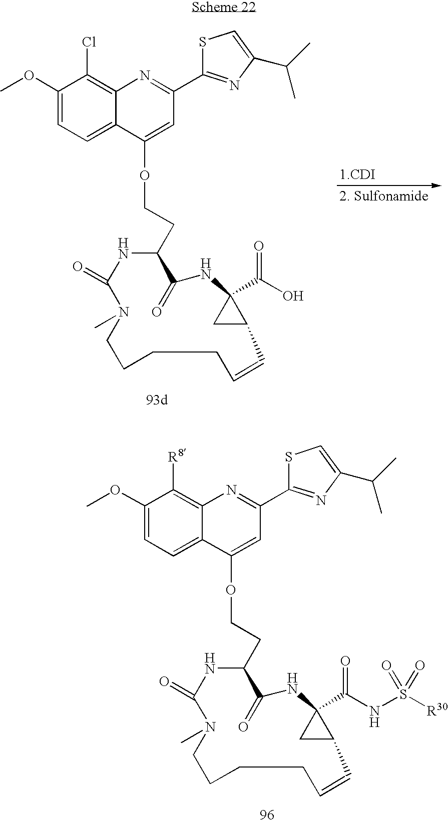

Step 1: Preparation of (1R,4S,14S,Z)-4-(2-(2-(4-isopropylthiazol-2-yl)-7-methoxy-8-chloro-quinolin-4-yloxy)ethyl)-7-methyl-N-(1-ethylcyclopropylsulfonyl)-3,6-dioxo-2,5,7-triazabicyclo[12.1.0]pentadec-12-ene-1-carboxamide 96a. Compound 96a was synthesized from compound 93d (100 mg, 1 eq.) and 1-ethylcyclopropylsulfonylamide 52a (68 mg, 3 eq.) as a white solid in 19% yield, following the procedure as described for compound 94b.

MS (ESI, EI⁺): m/z=787 (MH⁺).

Step 2: Preparation of (1R,4S,14S,Z)-4-(2-(2-(4-isopropylthiazol-2-yl)-7-methoxy-8-chloro-quinolin-4-yloxy)ethyl)-7-methyl-N-(1-cyclopropylmethyl-cyclopropylsulfonyl)-3,6-dioxo-2,5,7-triazabicyclo[12.1.0]pentadec-12-ene-1-carboxamide 96b. Compound 96b was synthesized from compound 93d (100 mg, 1 eq.) and 1-cyclopropylmethylcyclopropylsulfonylamide 52b (49 mg, 2 eq.) as an off-white solid in 6% yield, following the procedure as described for compound 94b.

MS (ESI, EI⁺): m/z=813 (MH⁺).

Step 3: Preparation of (1R,4S,14S,Z)-4-(2-(2-(4-isopropylthiazol-2-yl)-7-methoxy-8-chloro-quinolin-4-yloxy)ethyl)-7-methyl-N-(1-fluoro-cyclopropylsulfonyl)-3,6-dioxo-2,5,7-triazabicyclo[12.1.0]pentadec-12-ene-1-carboxamide 96c. Compound 96c was synthesized from compound 93d and 1-fluorocyclopropylsulfonylamide 52c as a white solid in 30% yield, following the procedure as described for compound 94b.

MS (ESI, EI⁺): m/z=778 (MH⁺).

Step 4: Preparation of (1R,4S,14S,Z)-4-(2-(2-(4-isopropylthiazol-2-yl)-7-methoxy-8-chloro-quinolin-4-yloxy)ethyl)-7-methyl-N-(1-cyano-cyclopropylsulfonyl)-3,6-dioxo-2,5,7-triazabicyclo[12.1.0]pentadec-12-ene-1-carboxamide 96d. Compound 96d was synthesized from compound 93d and 1-cyanocyclopropylsulfonylamide 52d as a white solid in 20% yield, following the procedure as described for compound 94b.

MS (ESI, EI⁺): m/z=785 (MH⁺).

Step 5: Preparation of (1R,4S,14S,Z)-4-(2-(2-(4-isopropylthiazol-2-yl)-7-methoxy-8-chloro-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 96e. Compound 96e was synthesized from compound 93d and 1-methylcyclopropylsulfonylamide as an off-white solid in 21% yield, following the procedure as described for compound 94b.

MS (ESI, EI⁺): m/z=773 (MH⁺).

Step 6: Preparation of (1R,4S,14S,Z)-4-(2-(2-(4-isopropylthiazol-2-yl)-7-methoxy-8-chloro-quinolin-4-yloxy)ethyl)-7-methyl-N-(cyclopropylsulfonyl)-3,6-dioxo-2,5,7-triazabicyclo[12.1.0]pentadec-12-ene-1-carboxamide 96f. Compound 96f was synthesized from compound 93d and cyclopropylsulfonylamide as an off-white solid in 10% yield, following the procedure as described for compound 94b.

MS (ESI, EI⁺): m/z=759 (MH⁺).

Step 7: Preparation of (1R,4S,14S,Z)-4-(2-(2-(4-isopropylthiazol-2-yl)-7-methoxy-8-chloro-quinolin-4-yloxy)ethyl)-7-methyl-N-(1-iodo-cyclopropylsulfonyl)-3,6-dioxo-2,5,7-triazabicyclo[12.1.0]pentadec-12-ene-1-carboxamide 96g. Compound 96g was synthesized from compound 93d (90 mg, 1 eq.) and 1-iodocyclopropylsulfonylamide (100 mg, 4 eq.) as a yellow solid in 12% yield, following the procedure as described for compound 94b.

MS (ESI, EI⁺): m/z=865 (MH⁺).

Step 8: Preparation of (1R,4S,14S,Z)-4-(2-(2-(4-isopropylthiazol-2-yl)-7-methoxy-8-chloro-quinolin-4-yloxy)ethyl)-7-methyl-N-(1-ethynylcyclopropylsulfonyl)-3,6-dioxo-2,5,7-triazabicyclo[12.1.0]pentadec-12-ene-1-carboxamide 96h. Compound 96h was synthesized from compound 93d and 1-ethynyl-clopropylsulfonylamide 52f as a white solid in 24% yield, following the procedure as described for compound 94b.

MS (ESI, EI⁺): m/z=784 (MH⁺).

Step 9: Preparation of (1R,4S,14S,Z)-4-(2-(8-chloro-2-(4-isopropylthiazol-2-yl)-7-methoxyquinolin-4-yloxy)ethyl)-N-(3,3-difluoropyrrolidin-1-ylsulfonyl)-7-methyl-3,6-dioxo-2,5,7-triazabicyclo[12.1.0]pentadec-12-ene-1-carboxamide 96i. Compound 96i was synthesized from compounds 57 (49 mg 3 eq.) and 93d (100 mg, 1 eq.) and (49 mg, 3 eq.) as a yellow solid in 11% yield, following the procedure as described for compound 94b.

MS (ESI, EI⁺): m/z=824 (MH⁺).

Step 10: Preparation of (1R,4S,14S,Z)-4-(2-(8-chloro-2-(4-isopropylthiazol-2-yl)-7-methoxyquinolin-4-yloxy)ethyl)-N—((S)-2-cyanopyrrolidin-1-ylsulfonyl)-7-methyl-3,6-dioxo-2,5,7-triazabicyclo[12.1.0]pentadec-12-ene-1-carboxamide 96k. Compound 96k was synthesized from compounds 93d (100 mg, 1 eq.) and 60 (66 mg, 2.5 eq.) as a white solid in 25% yield, following the procedure as described for compound 94b.

MS (ESI, EI⁺): m/z=813 (MH⁺).

Step 11: Preparation of (1R,4S,14S,Z)-4-(2-(8-chloro-2-(4-isopropylthiazol-2-yl)-7-methoxyquinolin-4-yloxy)ethyl)-N—((S)-2-ethynylpyrrolidin-1-ylsulfonyl)-7-methyl-3,6-dioxo-2,5,7-triazabicyclo[12.1.0]pentadec-12-ene-1-carboxamide 961. Compound 961 was synthesized from compounds 93d (100 mg, 1 eq.) and 65 (106 mg, 4 eq.) as an off-white solid in 25% yield, following the procedure as described for compound 94b.

MS (ESI, EI⁺): m/z=812 (MH⁺).

Step 12: Preparation of (1R,4S,14S,Z)-4-(2-(8-chloro-2-(4-isopropylthiazol-2-yl)-7-methoxyquinolin-4-yloxy)ethyl)-N—(N,N-dimethylsulfamoyl)-7-methyl-3,6-dioxo-2,5,7-triazabicyclo[12.1.0]pentadec-12-ene-1-carboxamide 96m. Compound 96m was synthesized from compound 93d and N,N-dimethylsulfamide as a beige solid in 47% yield.

MS (ESI, EI⁺): m/z=762 (MH⁺).

Step 13: Preparation of (1R,4S,14S,Z)-4-(2-(8-chloro-2-(4-isopropylthiazol-2-yl)-7-methoxyquinolin-4-yloxy)ethyl)-7-methyl-N-(morpholinosulfonyl)-3,6-dioxo-2,5,7-triazabicyclo[12.1.0]pentadec-12-ene-1-carboxamide 96n. Compound 96n was synthesized from compound 93d and 67 as a yellow solid in 28% yield.

MS (ESI, EI⁺): m/z=804 (MH⁺).

### Example 20

### Preparation of Macrocyclic Compound 100

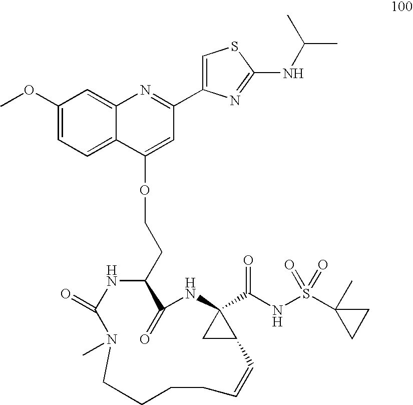

The synthesis of compound 100 is illustrated in Scheme 23.

Step 1: Preparation of (1R,4S,14S,Z)-ethyl 4-(2-(2-(2-(isopropylamino)thiazol-4-yl)-7-methoxyquinolin-4-yloxy)ethyl)-7-methyl-3,6-dioxo-2,5,7-triazabicyclo[12.1.0]pentadec-12-ene-1-carboxylate 98. Compound 98 was synthesized from compounds 48 (500 mg, 1 eq.) and 2-(2-isopropylamino-thiazol-4-yl)-7-methoxy-quinolin-4-ol (429 mg, 1 eq.) as a brown solid in 40% yield, following the procedure as described for compound 92b.

MS (ESI, EI⁺): m/z=665 (MH⁺).

Step 2: Preparation of (1R,4S,14S,Z)-4-(2-(2-(2-(isopropylamino)thiazol-4-yl)-7-methoxyquinolin-4-yloxy)ethyl)-7-methyl-3,6-dioxo-2,5,7-triazabicyclo[12.1.0]pentadec-12-ene-1-carboxylic acid 99. Compound 99 was synthesized from compound 98 as a yellow solid in 56% yield, following the procedure as described for compound 93b.

MS (ESI, EI⁺): m/z=637 (MH⁺).

Step 3: Preparation of (1R,4S,14S,Z)-4-(2-(2-(2-(isopropylamino)thiazol-4-yl)-7-methoxyquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 100. Compound 100 was synthesized from compound 98 and 1-methyl-cyclopropylsulfonamide as a yellow solid in 24% yield, following the procedure as described for compound 94b.

MS (ESI, EI⁺): m/z 754 (MH⁺).

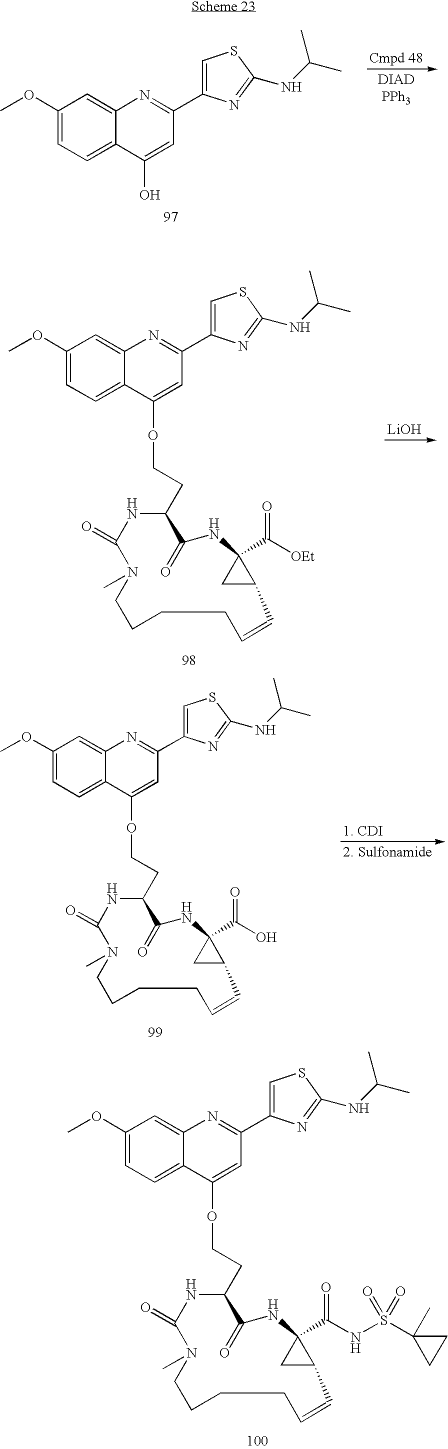

### Example 21

### Preparation of Macrocyclic Compound 103

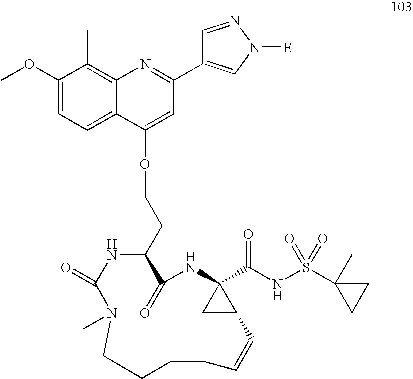

The syntheses of macrocyclic compounds 103 are illustrated in Scheme 24, wherein E in compounds 83, 101, and 102 is the same as compounds 103.

Step 1: Preparation of (1R,4S,14S,Z)-ethyl 4-(2-(7-methoxy-8-methyl-2-(1-ethyl-1H-pyrazol-4-yl)quinolin-4-yloxy)ethyl)-7-methyl-3,6-dioxo-2,5,7-triazabicyclo[12.1.0]pentadec-12-ene-1-carboxylate 101a. Compound 101a was synthesized from compounds 48 and 83a as a beige solid, following the procedure as described for compound 92b.

MS (ESI, EI⁺): m/z=633 (MH⁺).

Step 2: Preparation of (1R,4S,14S,Z)-ethyl 4-(2-(7-methoxy-8-methyl-2-(1-(2-morpholinoethyl)-1H-pyrazol-4-yl)quinolin-4-yloxy)ethyl)-7-methyl-3,6-dioxo-2,5,7-triazabicyclo[12.1.0]pentadec-12-ene-1-carboxylate 101b. Compound 101b was synthesized from compounds 48 and 83b as a beige solid, following the procedure as described for compound 92b.

MS (ESI, EI⁺): m/z=718 (MH⁺).

Step 3: Preparation of (1R,4S,14S,Z)-ethyl 4-(2-(7-methoxy-8-methyl-2-(1-isopenty-1H-pyrazol-4-yl)quinolin-4-yloxy)ethyl)-7-methyl-3,6-dioxo-2,5,7-triazabicyclo[12.1.0]pentadec-12-ene-1-carboxylate 101c. Compound 101c was synthesized from compounds 48 and 83c as a white solid in 60% yield, following the procedure as described for compound 92b.

MS (ESI, EI⁺): m/z 692 (MH⁺).

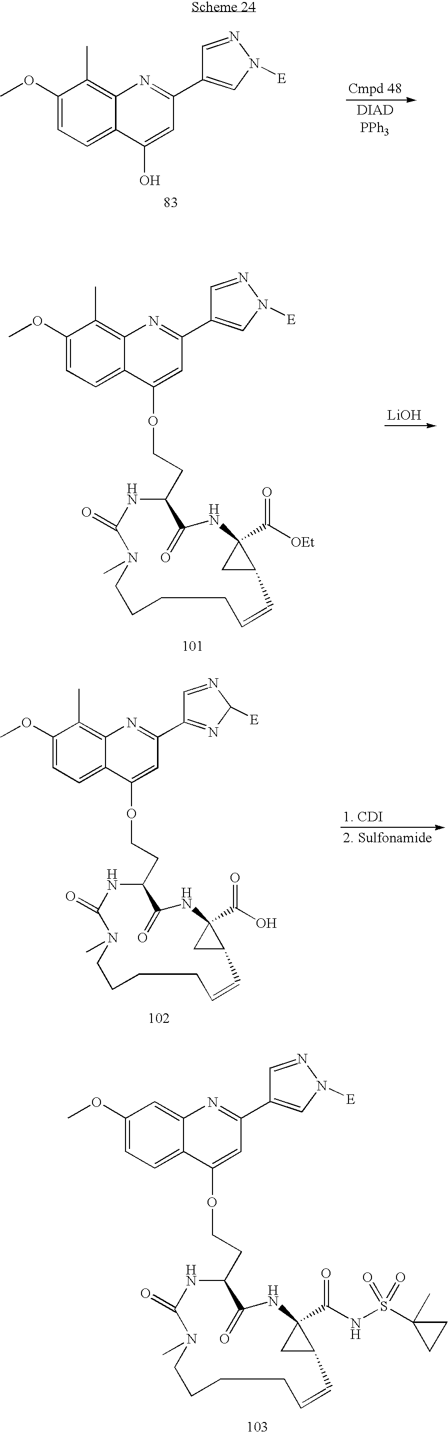

Step 4: Preparation of (1R,4S,14S,Z)-ethyl 4-(2-(7-methoxy-8-methyl-2-(1-benzyl-1H-pyrazol-4-yl)quinolin-4-yloxy)ethyl)-7-methyl-3,6-dioxo-2,5,7-triazabicyclo[12.1.0]pentadec-12-ene-1-carboxylate 101e. Compound 101e was synthesized from compounds 48 and 83e as a beige solid in 60% yield, following the procedure as described for compound 92b.

MS (ESI, EI⁺): m/z=695 (MH⁺).

Step 5: Preparation of (1R,4S,14S,Z)-ethyl 4-(2-(7-methoxy-8-methyl-2-(1-isobutyl-1H-pyrazol-4-yl)quinolin-4-yloxy)ethyl)-7-methyl-3,6-dioxo-2,5,7-triazabicyclo[12.1.0]pentadec-12-ene-1-carboxylate 101f. Compound 110f was synthesized from compounds 48 and 83f as a white solid in 60% yield, following the procedure as described for compound 92b.

MS (ESI, EI⁺): m/z=661 (MH⁺).

Step 6: Preparation of (1R,4S,14S,Z)-ethyl 4-(2-(7-methoxy-8-methyl-2-(1-propyl-1H-pyrazol-4-yl)quinolin-4-yloxy)ethyl)-7-methyl-3,6-dioxo-2,5,7-triazabicyclo[12.1.0]pentadec-12-ene-1-carboxylate 101g. Compound 101g was synthesized from compounds 48 and 83g as a beige solid, following the procedure as described for compound 92b.

MS (ESI, EI⁺): m/z=647 (MH⁺).

Step 7: Preparation of (1R,4S,14S,Z)-4-(2-(2-(1-ethyl-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102a. Compound 102a was synthesized from compound 110a as a beige solid, following the procedure as described for compound 93b.

MS (ESI, EI⁺): m/z=606 (MH⁺).

Step 8: Preparation of (1R,4S,14S,Z)-4-(2-(2-(1-(2-morpholinoethyl)-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102b. Compound 102b was synthesized from compound 101b as an off-white solid in 24% yield, following the procedure as described for compound 93b.

MS (ESI, EI⁺): m/z=690 (MH⁺).

Step 9: Preparation of (1R,4S,14S,Z)-4-(2-(2-(1-isopenty-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102c. Compound 102c was synthesized from compound 101c as a white solid in 70% yield, following the procedure as described for compound 93b.

MS (ESI, EI⁺): m/z=663 (MH⁺).

Step 10: Preparation of (1R,4S,14S,Z)-4-(2-(2-(1-benzyl-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102e. Compound 102e was synthesized from compound 101e as a beige solid, following the procedure as described for compound 93b.

MS (ESI, EI⁺): m/z=671 (MH⁺).

Step 11: Preparation of (1R,4S,14S,Z)-4-(2-(2-(1-isobutyl-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102f. Compound 102f was synthesized from compound 110f as a white solid in 70% yield, following the procedure as described for compound 93b.

MS (ESI, EI⁺): m/z=632 (MH⁺).

Step 12: Preparation of (1R,4S,14S,Z)-4-(2-(2-(1-propyl-1H-pyrazol-4-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102g. Compound 102g was synthesized from compound 101g as an off-white solid in 23% yield, following the procedure as described for compound 93b.

MS (ESI, EI⁺): m/z=619 (MH⁺).

Step 13: Preparation of (1R,4S,14S,Z)-4-(2-(2-(1-ethyl-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103a. Compound 103a was synthesized from compound 102a and 1-methylcyclopropylsulfonamide as a white solid, following the procedure as described for compound 94b.

MS (ESI, EI⁺): m/z=722 (MH⁺).

Step 14: Preparation of (1R,4S,14S,Z)-4-(2-(2-(1-(2-morpholinoethyl)-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103b. Compound 103b was synthesized from compound 102b and 1-methylcyclopropylsulfonamide as a beige solid in 10% yield, following the procedure as described for compound 94b.

MS (ESI, EI⁺): m/z=807 (MH⁺).

Step 15: Preparation of (1R,4S,14S,Z)-4-(2-(2-(1-isopentyl-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103c. Compound 103c was synthesized from compound 102c and 1-methylcyclopropylsulfonamide as a white solid in 15% yield, following the procedure as described for compound 94b.

MS (ESI, EI⁺): m/z=764 (MH⁺).

Step 16: Preparation of (1R,4S,14S,Z)-4-(2-(2-(1-benzyl-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103e. Compound 103e was synthesized from compound 102e and 1-methylcyclopropylsulfonamide as a white solid, following the procedure as described for compound 94b.

MS (ESI, EI⁺): m/z=784 (MH⁺).

Step 17: Preparation of (1R,4S,14S,Z)-4-(2-(2-(1-isobutyl-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103f. Compound 103f was synthesized from compound 102f and 1-methylcyclopropylsulfonamide as a white solid in 15% yield, following the procedure as described for compound 94b.

MS (ESI, EI⁺): m/z=750 (MH⁺).

Step 18: Preparation of (1R,4S,14S,Z)-4-(2-(2-(1-propyl-1H-pyrazol-4-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103g. Compound 103g was synthesized from compound 102g and 1-methylcyclopropylsulfonamide as a white solid in 7% yield, following the procedure as described for compound 94b.

MS (ESI, EI⁺): m/z=736 (MH⁺).

### Example 22

### Preparation of Macrocyclic Compound 106

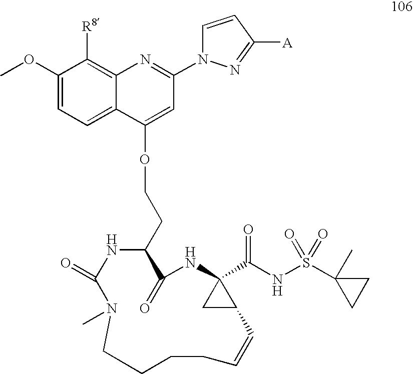

The syntheses of macrocyclic compound 106 is illustrated in Scheme 25.

Step 1: Preparation of (1R,4S,14S,Z)-ethyl 4-(2-(7-methoxy-8-methyl-2-(3-(trifluoromethyl)-1H-pyrazol-1-yl)quinolin-4-yloxy)ethyl)-7-methyl-3,6-dioxo-2,5,7-triazabicyclo[12.1.0]pentadec-12-ene-1-carboxylate 104a. Compound 104a was synthesized from compounds 48 and 91a as a beige solid, following the procedure as described for compound 92b.

MS (ESI, EI⁺): m/z=673 (MH⁺).

Step 2: Preparation of (1R,4S,14S,Z)-ethyl 4-(2-(7-methoxy-8-chloro-2-(3-(trifluoromethyl)-1H-pyrazol-1-yl)quinolin-4-yloxy)ethyl)-7-methyl-3,6-dioxo-2,5,7-triazabicyclo[12.1.0]pentadec-12-ene-1-carboxylate 104b. Compound 104b was synthesized from compounds 48 and 91b as a beige solid in 50% yield, following the procedure as described for compound 92b.

MS (ESI, EI⁺): m/z=693 (MH⁺).

Step 3: Preparation of (1R,4S,14S,Z)-ethyl 4-(2-(7-methoxy-8-chloro-2-(3-isopropyl-1H-pyrazol-1-yl)quinolin-4-yloxy)ethyl)-7-methyl-3,6-dioxo-2,5,7-triazabicyclo[12.1.0]pentadec-12-ene-1-carboxylate 104c. Compound 104c was synthesized from compounds 48 and 91c as a beige solid in 87% yield, following the procedure as described for compound 92b.

MS (ESI, EI⁺): m/z=667 (MH⁺).

Step 4: Preparation of (1R,4S,14S,Z)-ethyl 4-(2-(7-methoxy-8-methyl-2-(3-isopropyl-1H-pyrazol-1-yl)quinolin-4-yloxy)ethyl)-7-methyl-3,6-dioxo-2,5,7-triazabicyclo[12.1.0]pentadec-12-ene-1-carboxylate 104d. Compound 104d was synthesized from compounds 48 and 91d as a white solid in 60% yield, following the procedure as described for compound 92b.

MS (ESI, EI⁺): m/z 647 (MH⁺).

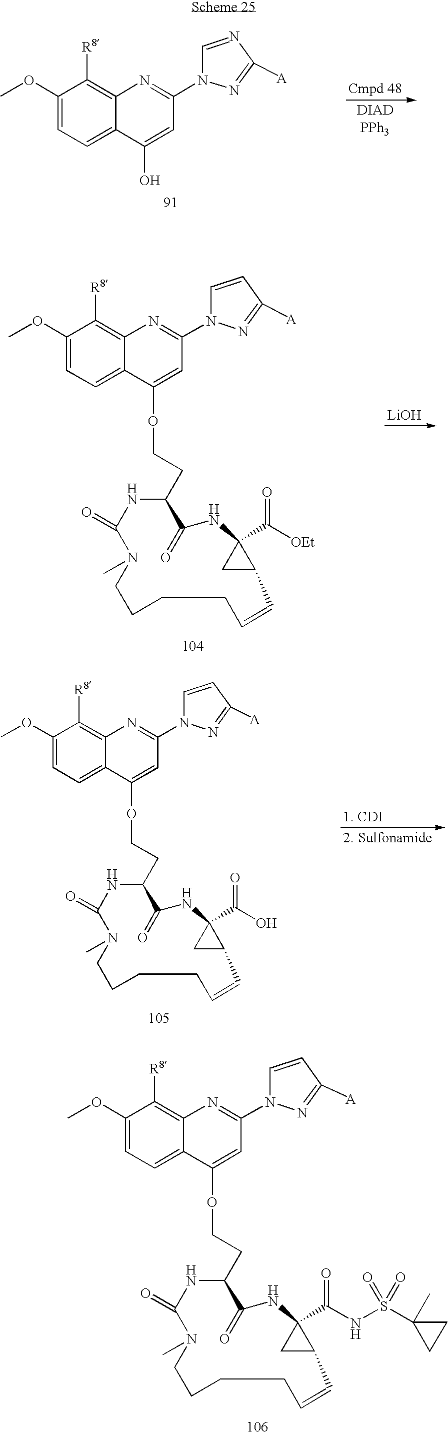

Step 5: Preparation of (1R,4S,14S,Z)-4-(2-(7-methoxy-8-methyl-2-(3-trifluoromethyl-1H-pyrazol-1-yl)quinolin-4-yloxy)ethyl)-7-methyl-3,6-dioxo-2,5,7-triazabicyclo[12.1.0]pentadec-12-ene-1-carboxylic acid 105a. Compound 105a was synthesized from compound 104a as a white solid, following the procedure as described for compound 93b.

MS (ESI, EI⁺): m/z=645 (MH⁺).

Step 6: Preparation of (1R,4S,14S,Z)-4-(2-(7-methoxy-8-chloro-2-(3-trifluoromethyl-1H-pyrazol-1-yl)quinolin-4-yloxy)ethyl)-7-methyl-3,6-dioxo-2,5,7-triazabicyclo[12.1.0]pentadec-12-ene-1-carboxylic acid 105b. Compound 105b was synthesized from compound 104b as a white solid in 92% yield, following the procedure as described for compound 93b.

MS (ESI, EI⁺): m/z=665 (MH⁺).

Step 7: Preparation of (1R,4S,14S,Z)-4-(2-(7-methoxy-8-chloro-2-(3-isopropy-1H-pyrazol-1-yl)quinolin-4-yloxy)ethyl)-7-methyl-3,6-dioxo-2,5,7-triazabicyclo[12.1.0]pentadec-12-ene-1-carboxylic acid 105c. Compound 105c was synthesized from compound 104c as a white solid in 59% yield, following the procedure as described for compound 93b.

MS (ESI, EI⁺): m/z=639 (MH⁺).

Step 8: Preparation of (1R,4S,14S,Z)-4-(2-(7-methoxy-8-methyl-2-(3-isopropy-1H-pyrazol-1-yl)quinolin-4-yloxy)ethyl)-7-methyl-3,6-dioxo-2,5,7-triazabicyclo[12.1.0]pentadec-12-ene-1-carboxylic acid 105d. Compound 105d was synthesized from compound 104d as a white solid in 75% yield, following the procedure as described for compound 93b.

MS (ESI, EI⁺): m/z=619 (MH⁺).

Step 9: Preparation of (1R,4S,14S,Z)-4-(2-(2-(3-trifluoromethyl-1H-pyrazol-1-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 106a. Compound 106a was synthesized from compound 105a and methyl-cyclopropylsulfonamide as a pale yellow solid, following the procedure as described for compound 94b.

MS (ESI, EI⁺): m/z=762 (MH⁺).

Step 10: Preparation of (1R,4S,14S,Z)-4-(2-(2-(3-trifluoromethyl-1H-pyrazol-1-yl)-7-methoxy-8-chloro-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 106b. To a stirred solution of compound 105b (120 mg, 1 eq.) and TEA (50 μL, 2 eq.) in DCM (18 mL) was added HATU (137 mg, 2 eq.). The reaction mixture was stirred at 40° C. for 1 hr. The mixture was then washed with water, brine, dried over Na₂SO₄, filtered, and concentrated under reduced pressure to yield an intermediate as a white solid, which was used in the next step without purification.

To a stirred solution of methyl-cyclopropylsulfonamide (55 mg, 2 eq.) in anhydrous THF (9 mL) was added NaH (60% in oil, 15.8 mg, 2.2 eq.) at room temperature. After 20 min, a solution of the intermediate (0.18 mmol) in anhydrous THF (2 mL) was added dropwise to the reaction mixture. The mixture was stirred at 80° C. for 1 hr. THF was evaporated. The residue obtained was dissolved in DCM, washed with water, dried over Na₂SO₄, filtered, concentrated under reduced pressure, and purified by silica gel chromatography (DCM/MeOH) to yield compound 106b as a white solid in 68% yield.

MS (ESI, EI⁺): m/z=782 (MH⁺).

Step 11: Preparation of (1R,4S,14S,Z)-4-(2-(2-(3-isopropyl-1H-pyrazol-1-yl)-7-methoxy-8-chloro-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 106c. Compound 105c first reacted with HATU to form an intermediate as a white solid, following the procedure as described for compound 106b. The intermediate then reacted with methyl-cyclopropylsulfonamide to form compound 106c as a white solid in 6% yield, following the procedure as described for compound 106b.

MS (ESI, EI⁺): m/z=756 (MH⁺).

Step 12: Preparation of (1R,4S,14S,Z)-4-(2-(2-(3-isopropyl-1H-pyrazol-1-yl)-7-methoxy-8-methyl-quinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 106d. Compound 106d was synthesized from compound 105d and 1-methyl-cyclorpopylsulfonamide as a white solid in 15% yield, following the procedure as described for compound 64b.

MS (ESI, EI⁺): m/z=736 (MH⁺).

### Example 23

### Preparation of Macrocyclic Compound 109

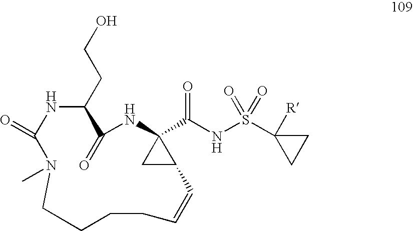

The syntheses of macrocyclic compound 109 is illustrated in Scheme 26.

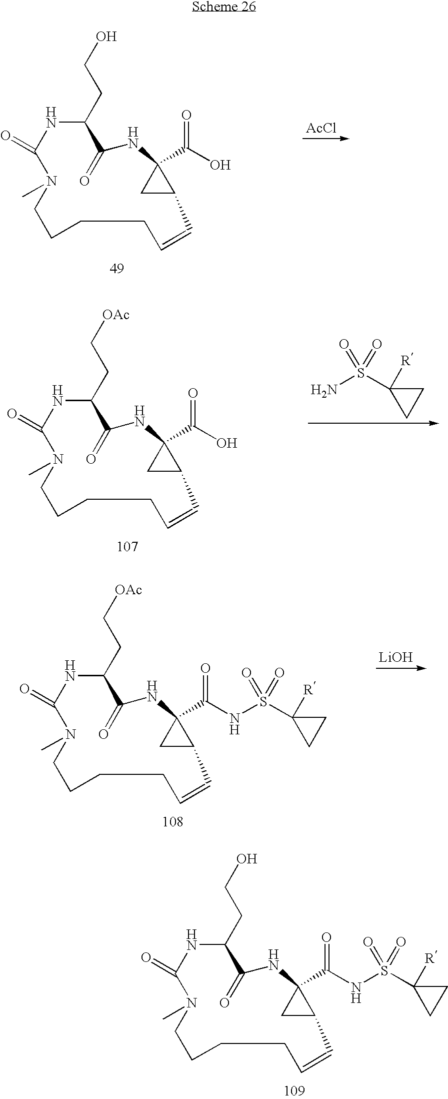

Step 1: Preparation of 4-(2-acetyl-ethyl)-7-methyl-3,6-dioxo-2,5,7-triazabicyclo[12.1.0]pentadec-12-ene-1-carboxylic acid 107. To a stirred solution of compound 49 (70 mg, 1 eq.) in DCM (5 mL) was added TEA (104 μL, 3.5 eq.) at 0° C. Acetyl chloride (30 μL, 2 eq.) was then added and the reaction mixture was stirred at room temperature for 3 hrs. AcOH (5 eq.) was added and solvents were removed under reduced pressure. The crude material was purified by silica gel chromatography to yield compound 107 as a beige solid in 57% yield.

MS (ESI, EI⁺): m/z=382 (MH⁺).

Step 2: Preparation of 4-(2-acetyl-ethyl)-7-methyl-3,6-dioxo-2,5,7-triazabicyclo[12.1.0]pentadec-12-ene-1-carbonyl cyclopropanesulfonamide 108a. Compound 108a was synthesized from compound 107 and cyclopropylsulfonamide as a white solid in 78% yield, following the procedure as described for compound 92a.

MS (ESI, EI⁺): m/z=485 (MH⁺).

Step 3: Preparation of 4-(2-acetyl-ethyl)-7-methyl-3,6-dioxo-2,5,7-triazabicyclo[12.1.0]pentadec-12-ene-1-carbonyl methyl-cyclopropanesulfonamide 108b. Compound 108b was synthesized from compound 107 and 1-methyl-cyclopropylsulfonamine as an off-white solid, following the procedure as described for compound 92a.

MS (ESI, EI⁻): m/z=483 (MH⁻).

Step 4: Preparation of 4-(hydroxy-ethyl)-7-methyl-3,6-dioxo-2,5,7-triazabicyclo[12.1.0]pentadec-12-ene-1-carbonyl cyclopropanesulfonamide 109a. To a stirred solution of compound 108a (110 mg, 1 eq.) in MeOH (5 mL) and water (1 mL) was added LiOH (11 mg, 2 eq.). The reaction mixture was stirred at room temperature for 2 hrs. AcOH was then added (100 μL). The mixture was concentrated under reduced pressure and the crude material was purified by silica gel chromatography to yield compound. 109a as a white solid in 36% yield.

MS (ESI, EI⁺): m/z=443 (MH⁺).

### Example 24

### Preparation of Macrocyclic Compound 114

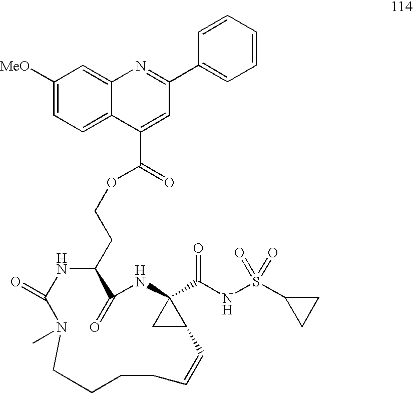

The syntheses of macrocyclic compound 114 is illustrated in Scheme 27.

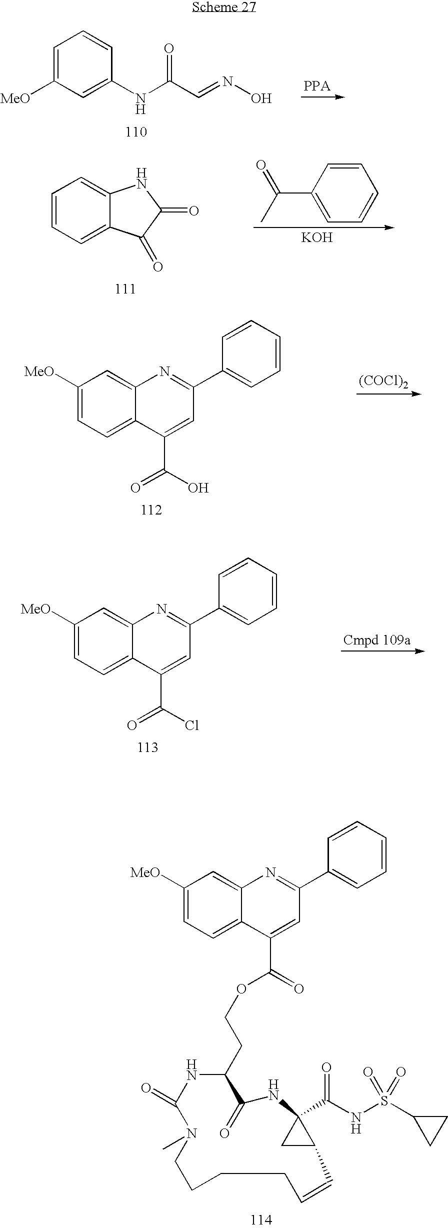

Step 1: Preparation of 1-(methoxy-benzyl)-hydroxyimino-acetamide 110. Compound 110 was synthesized from m-anisidine as a brown solid in 97% yield, following the procedure as described in WO 03/055866.

¹H NMR (DMSO-d₆, 400 MHz) δ 3.71 (s, 3H), 6.64-6.67 (m, 1H), 7.20-7.22 (m, 2H), 7.33 (m, 1H), 7.63 (s, 1H), 10.14 (s, 1H), 12.21 (s, 1H).

Step 2: Preparation of 6-methoxy-1H-indole-2,3-dione 111. Compound 110 (15 g, 1 eq.) was stirred with polyphosphoric acid (150g) at 80° C. for 10 min and then poured into ice/water. Aqueous layer was extracted with DCM. Organics were dried over Na₂SO₄, filtered, and concentrated under reduced pressure to yield compound III as an orange solid in 27% yield.

MS (ESI, EI⁺): m/z=178 (MH⁺).

Step 3: Preparation of 7-methoxy-2-phenyl-quinoline-4-carboxylic acid 112. Compound III (500 mg, 1 eq.) and acetophenone (380 μL, 1.2 eq.) were added at room temperature to a solution of KOH (520 mg, 3.3 eq.) in ethanol (5 mL). The reaction mixture was stirred at 70° C. for 7 hrs. The mixture was then poured into ice/water, and washed with dichloromethane. Aqueous layer was acidified with 3N HCl to pH 2-3. The precipitate obtained was filtered, washed with water, and triturated in ethanol to yield compound 112 as a beige solid in 40% yield.

MS (ESI, EI⁺): m/z=280 (MH⁺).

Step 4: Preparation of 7-methoxy-2-phenyl-quinoline-4-carbonyl chloride 113. To a stirred solution of compound 112 (45 mg, 1 eq.) in DCM (4 mL) at 0° C. was added oxalyl chloride (81 mg, 4 eq.). A few drops of DMF were added. The reaction mixture was stirred at room temperature for 3 hrs. The mixture was concentrated under reduced pressure and co-evaporated with hexane to yield compound 112 as a white solid in 60%.

MS (ESI, EI⁺): m/z=298 (MH⁺).

Step 5: Preparation of (1R,4S,14S,Z)-N-(cyclopropylsulfonyl)-4-(2-(7-methoxy-2-phenylquinolin-4-yloxy)ethyl)-7-methyl-3,6-dioxo-2,5,7-triazabicyclo[12.1.0]pentadec-12-ene-1-carboxamide 114. To a stirred solution of compound 109a (35 mg, 1 eq.) in DCM (3 mL) at 0° C. was added compound 114 (2 eq.) and TEA (33 μL, 3 eq.). The reaction mixture was stirred at room temperature for 16 hrs. MeOH was then added, the mixture was concentrated under reduced pressure and purified by silica gel chromatography to yield compound 114 as a pale yellow powder in 20% yield.

MS (ESI, EI⁺): m/z=704 (MH⁺).

### Example 25

### Preparation of Macrocyclic Compound 103h

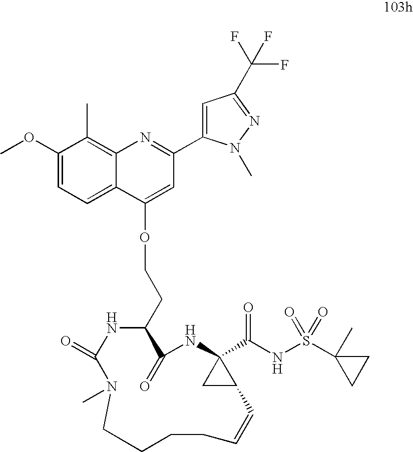

The syntheses of macrocyclic compound 103h is illustrated in Scheme 28.

Step 1: Preparation of (1R,4S,14S,Z)-ethyl 4-(2-(7-methoxy-8-methyl-2-(1-methyl-3-(trifluoromethyl)-1H-pyrazol-5-yl)quinolin-4-yloxy)ethyl)-7-methyl-3,6-dioxo-2,5,7-triazabicyclo[12.1.0]pentadec-12-ene-1-carboxylate 101h. Compound 101h was synthesized from compounds 48 and 85 as a white solid in 84% yield, following the procedure as described for compound 92b.

MS (ESI, EI⁺): m/z=687 (MH⁺).

Step 2: Preparation of (1R,4S,14S,Z)-4-(2-(2-(1-methyl-3-(trifluoromethyl)-1H-pyrazol-5-yl)-7-methoxy-8-methylquinolin-4-yloxy)ethyl)-7-methyl-3,6-dioxo-2,5,7-triazabicyclo[12.1.0]pentadec-12-ene-1-carboxylic acid 102h. Compound 102h was synthesized from compound 101h as a white solid in 45% yield, following the procedure as described for compound 93b.

MS (ESI, EI⁺): m/z=659 (MH⁺).

Step 3: Preparation of (1R,4S,14S,Z)-4-(2-(2-(1-methyl-3-(trifluoromethyl)-1H-pyrazol-5-yl)-7-methoxy-8-methylquinolin-4-yloxy)ethyl)-7-methyl-N-(1-methylcyclopropylsulfonyl)-3,6-dioxo-2,5,7-triazabicyclo[12.1.0]pentadec-12-ene-1-carboxamide 103h. Compound 103h was synthesized from compound 102h (210 mg, 1 eq.) and 1-methylcyclopropylsulfonamide (172 mg, 0.4 mmol) as a white solid in 15% yield, following the procedure as described for compound 94b.

MS (ESI, EI⁺): m/z=776 (MH⁺).

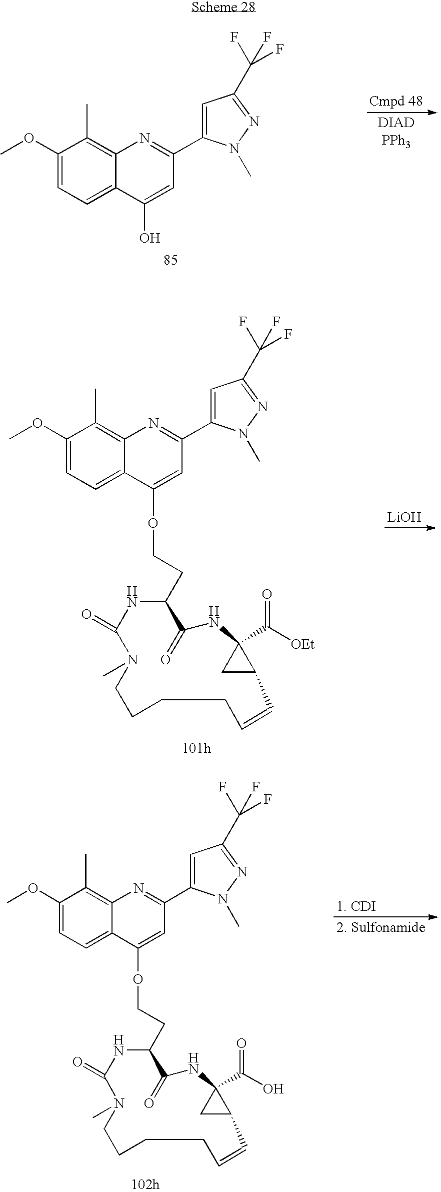

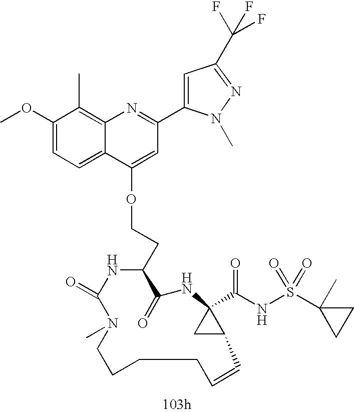

### Example 26

### Preparation of Substituted Quinolines 91

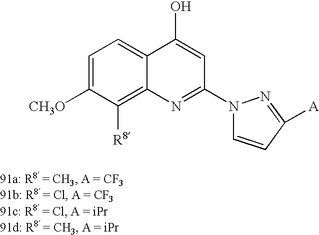

The syntheses of substituted quinolines are illustrated in Scheme 29, where R^{8′} and A in compound 104 are the same as defined in compound 91.

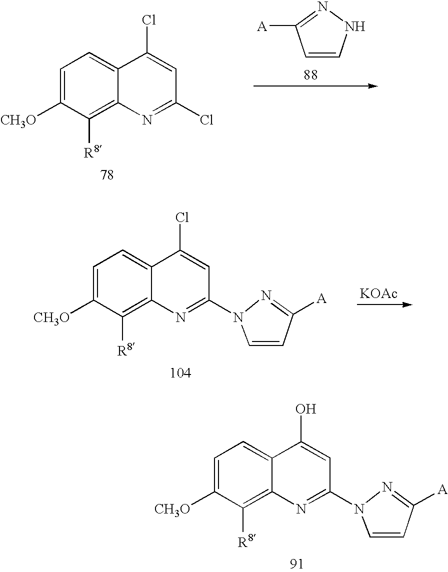

Step 1: Synthesis of 4,8-dichloro-7-methoxy-2-(3-(trifluoromethyl)-1H-pyrazol-1-yl)quinoline 104b. A mixture of compound 78d (5 g, 19 mmol) and 3-trifluoromethylpyrazole 88a (7.76 g, 57 mmol) was heated at 120° C. for 4-6 hrs and the reaction was followed by LCMS and TLC. The reaction mixture was purified by silica gel column (mono and dipyrazole were separated) using DCM and heptane as mobile phase to yield compound 104b (3.5 g) in 51% yield.

Step 2: Synthesis of 8-chloro-7-methoxy-2-(3-(trifluoromethyl)-1H-pyrazol-1-yl)quinolin-4-ol 91b. To a solution of compound 104b (250 mg) in DMSO (2.5 mL) was added CH₃COOK (3 eq.), water (2 eq.). The reaction mixture was heated to 140° C. for 4 hrs. After cooled to RT, water (1 mL) was added to the reaction mixture slowly under stirring. Solid was filtered and washed with water to yield compound 91b in >80% yield. In a separate reaction, when 5 eq. of CH₃COOK was used, the reaction was completed in 1 hr.

### Example 27

### HCV Protease Assay

General procedure: Measurement of the inhibitory effect of compounds on HCV protease activity was performed with the SensoLyte™ 620 HCV Protease Assay kit from AnaSpec, Inc. (San Jose, Calif.) under conditions described by the supplier using 1.2 nM HCV NS3-NS4A protease, which was obtained according to Taremi et al. (Protein Science, 1998, 7, 2143-2149). The compounds were tested at a variety of concentrations in assay buffer containing a final DMSO concentration of 5%. Reactions were allowed to proceed for 60 min at room temperature and fluorescence measurements were recorded with a Tecan Infinity Spectrofluorimeter. The IC₅₀ values were determined from the percent inhibition versus concentration data using a sigmoidal non-linear regression analysis based on four parameters with Tecan Magellan software.

### Example 28

### HCV Replicon Assay

General procedure: Huh-7 cells containing HCV Con1 subgenomic replicon (GS4.1 cells) were grown in Dulbecco's Modified Eagle Medium (DMEM) supplemented with 10% fetal bovine serum (FBS), 2 mM L-glutamine, 110 mg/L sodium pyruvate, 1X non-essential amino acids, 100 U/mL penicillin-streptomycin, and 0.5 mg/mL G418 (Invitrogen). For dose-response testing, the cells were seeded in 96-well plates at 7.5×10³ cells/well in a volume of 50 μL, and incubated at 37° C./5% CO₂. Three hours after plating, 50 μL of ten 2-fold serial dilutions of compounds (highest concentration, 75 μM) were added, and cell cultures were incubated at 37° C./5% CO₂ in the presence of 0.5% DMSO. Alternatively, compounds were tested at a single concentration of 15 μM. In all cases, Huh-7 cells lacking the HCV replicon served as negative control. The cells were incubated in the presence of compounds for 72 hr after which they were monitored for expression of the NS4A protein by enzyme-linked immunosorbent assay (ELISA). For this, the plates were then fixed for 1 min with acetone/methanol (1:1, v/v), washed twice with phosphate-buffered saline (PBS), 0.1% Tween 20, blocked for 1 hr at room temperature with TNE buffer containing 10% FBS and then incubated for 2 hr at 37° C. with the anti-NS4A mouse monoclonal antibody A-236 (ViroGen) diluted in the same buffer. After washing three times with PBS, 0.1% Tween 20, the cells were incubated 1 hr at 37° C. with anti-mouse immunoglobulin G-peroxidase conjugate in TNE, 10% FBS. After washing as described above, the reaction was developed with O-phenylenediamine (Zymed). The reaction was stopped after 30 min with 2 N H₂SO₄, and absorbance was read at 492 nm using Sunrise Tecan spectrophotometer. EC₅₀ values were determined from the % inhibition versus concentration data using a sigmoidal non-linear regression analysis based on four parameters with Tecan Magellan software. When screening at a single concentration, the results were expressed as % inhibition at 15 μM.

For cytotoxicity evaluation, GS4.1 cells were treated with compounds as described above and cellular viability was monitored using the Cell Titer 96 AQᵤₑₒᵤₛ One Solution Cell Proliferation Assay (Promega). CC₅₀ values were determined from the % cytotoxicity versus concentration data with Tecan Magellan software as described above.

The biological results are summarized in Table 3, wherein A represents a value smaller than 100 nM, B represents a value between 100 nM to 10 μM, and C represents a value between than 10 μM to 1 mM, and D represents a value greater than 1 mM.

TABLE 3

| | IC₅₀ | EC₅₀ | CC₅₀ |
|---|---|---|---|
| Compound | (μM) | (μM) | (μM) |
| 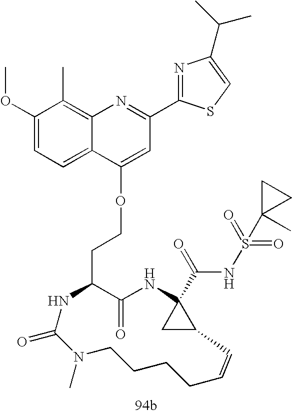 | A | A | D |
| 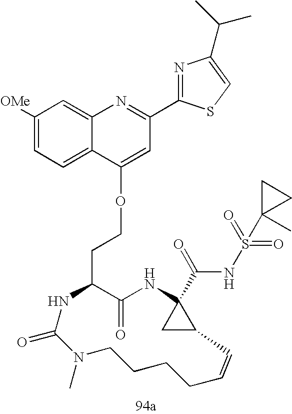 | A | A | C |
| 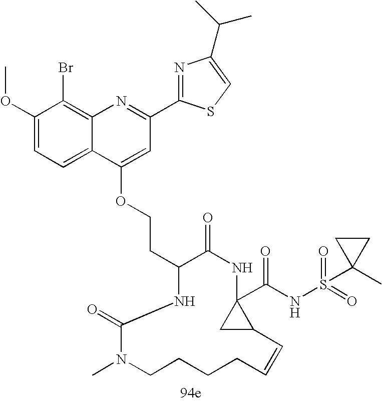 | A | A | D |
| 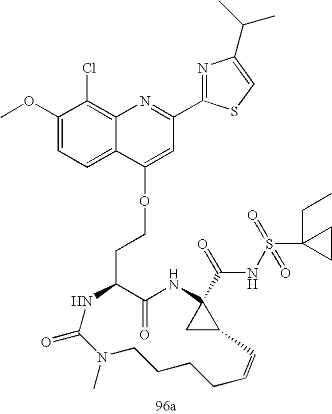 | A | A | D |
| 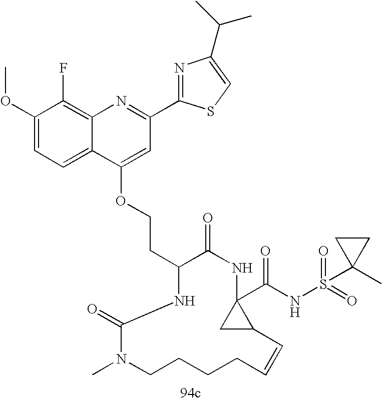 | A | B | D |
| 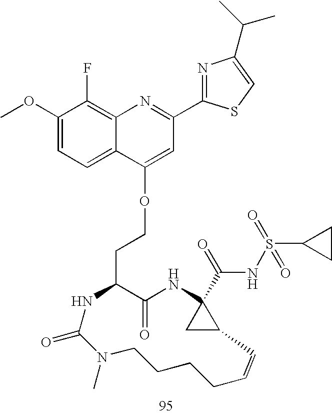 | A | C | D |
| 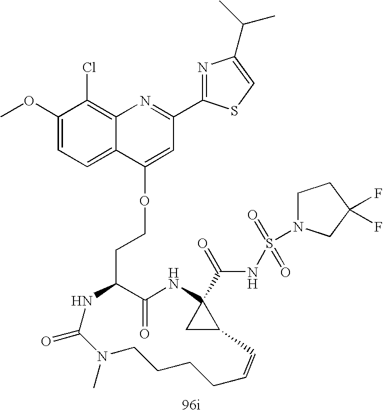 | B | B | D |
| 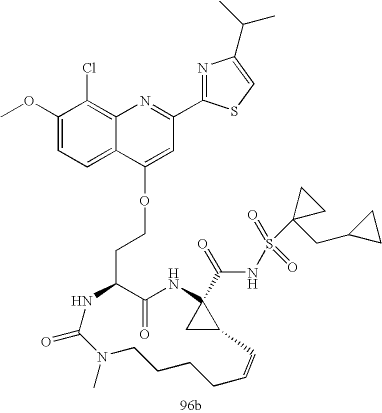 | A | B | D |
| 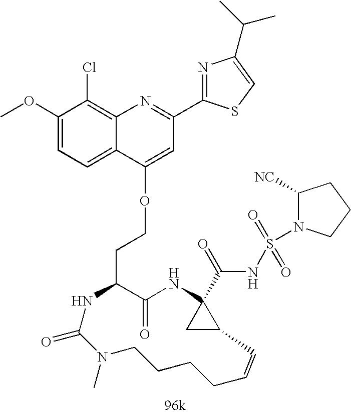 | B | B | D |
| 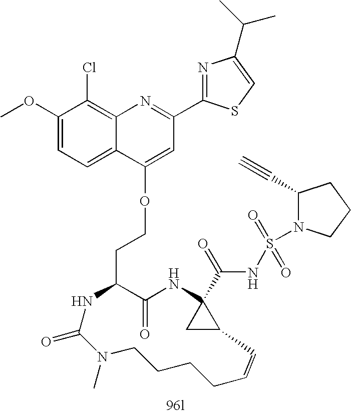 | A | B | D |
| 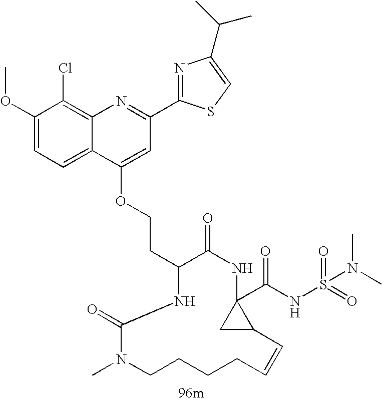 | A | A | D |
| 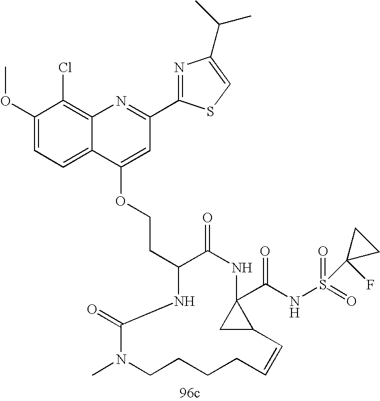 | A | B | D |
| 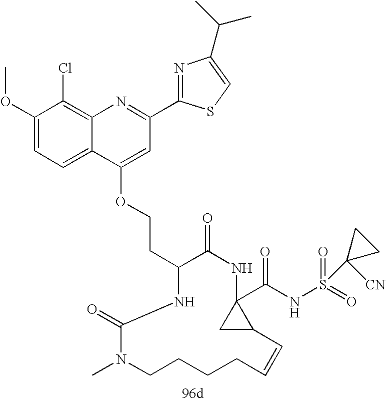 | A | B | D |
| 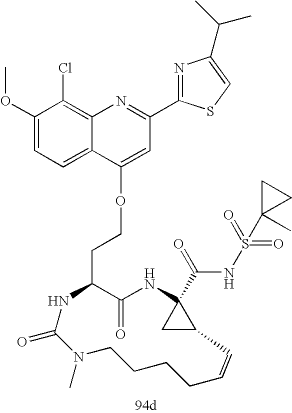 | A | A | D |
| 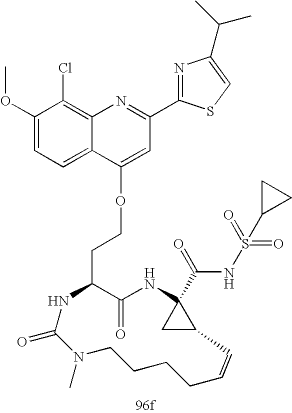 | A | A | D |
| 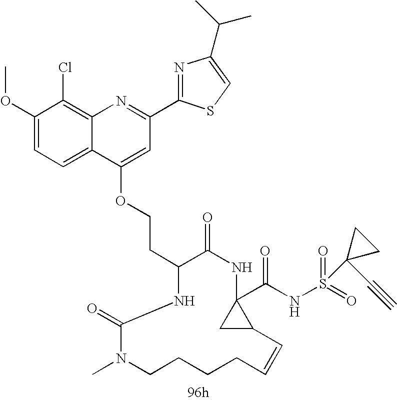 | A | A | D |
| 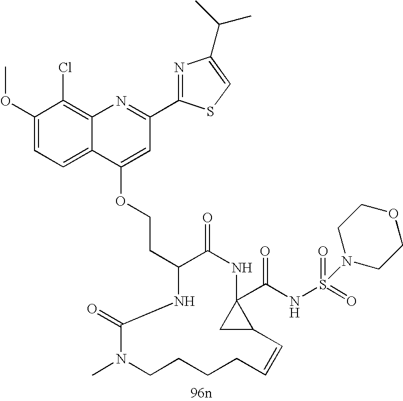 | B | B | D |
| 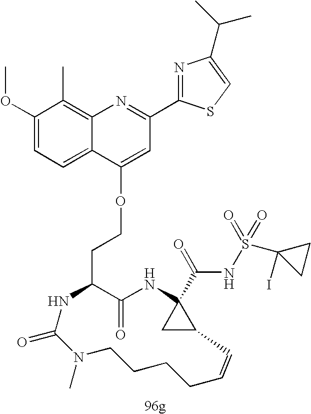 | A | B | D |
| 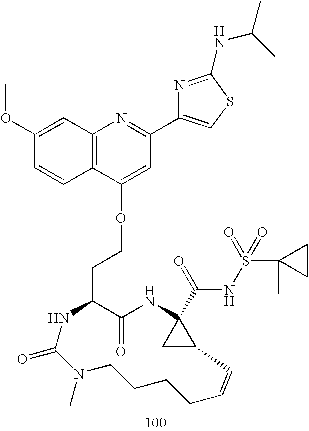 | A | B | D |
| 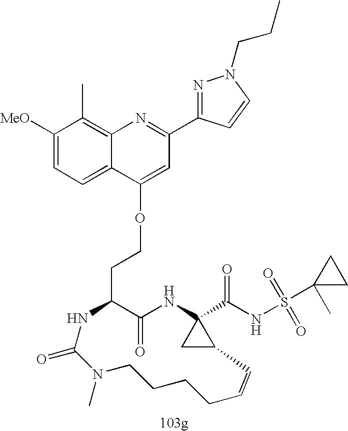 | A | B | D |
| 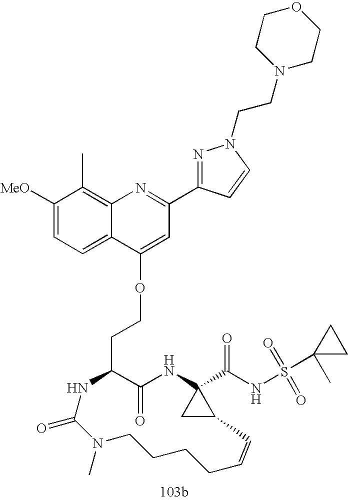 | A | B | D |
| 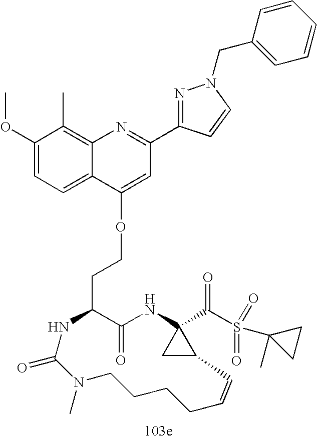 | A | B | D |
| 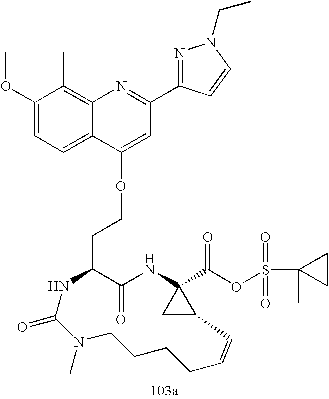 | A | B | D |
| 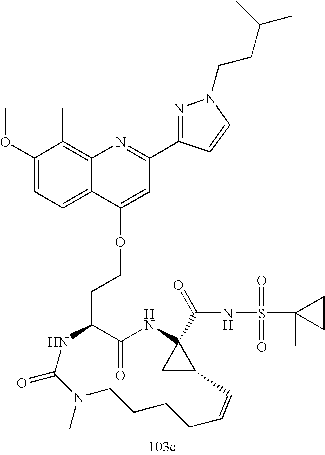 | A | B | D |
| 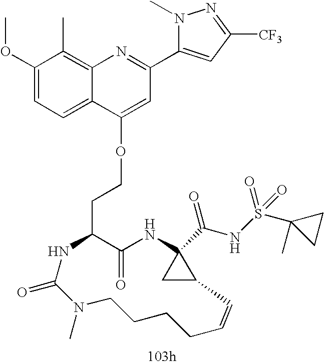 | A | B | D |
| 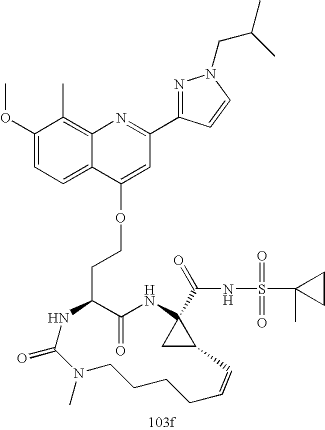 | A | B | D |
| 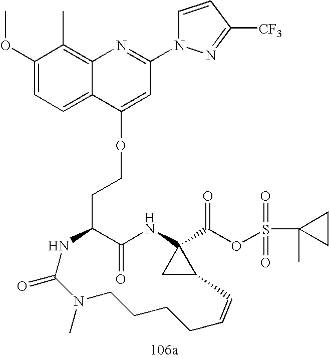 | A | A | D |
| 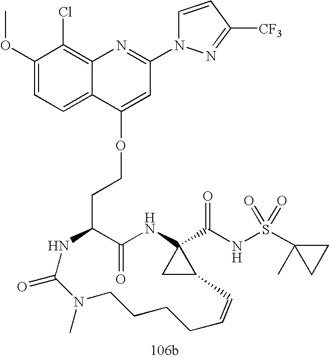 | A | A | D |
| 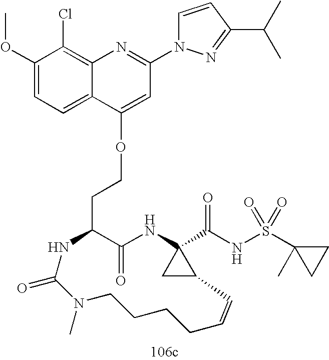 | A | A | D |
| 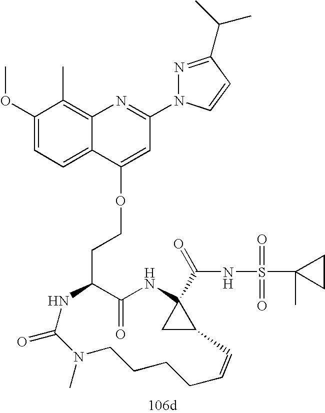 | A | A | D |
| 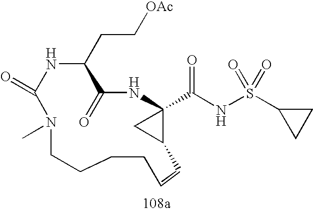 | B | D | D |
| 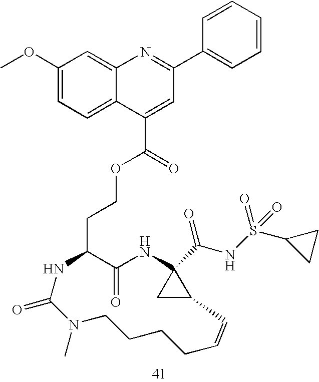 | B | D | D |
| 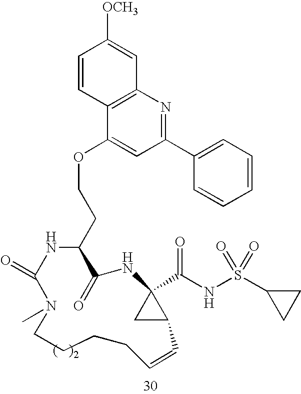 | | C | C |
| 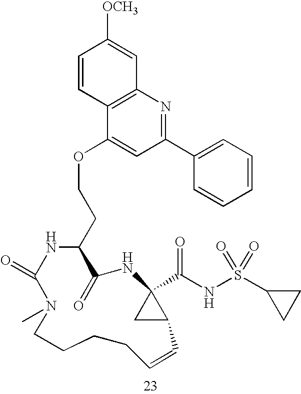 | A | B | D |
| 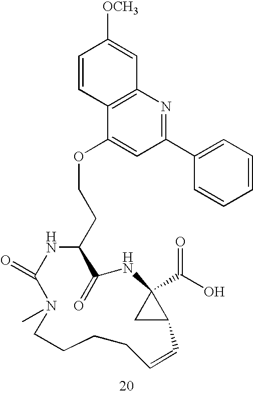 | B | C | D |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula I: 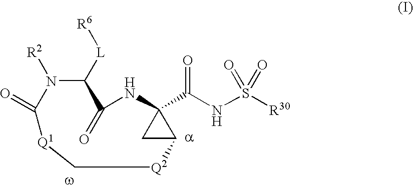
or a single enantiomer, a mixture of enantiomers, an individual diastereomer, or a mixture of diastereomers thereof; or a pharmaceutically acceptable salt, solvate, or prodrug thereof;
wherein:
R² is hydrogen, C₁₋₆ alkyl, C₃₋₇ cycloalkyl, C₆₋₁₄ aryl, heterocyclyl, or heteroaryl;
R⁶ is hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl;
R³⁰ is hydrogen, C₁₋₆ alkyl, C₂₋₆ alkenyl, C₂₋₆ alkynyl, C₃₋₇ cycloalkyl, C₆₋₁₄ aryl, heteroaryl, heterocyclyl, or C₁₋₆ alkyl-C₃₋₇ cycloalkylene;
L is a bond, C₁₋₆ alkylene, C₂₋₆ alkenylene, C₂₋₆ alkynylene, C₃₋₇ cycloalkyl, or —(CR^{a}R^{b})ₚX—; wherein p is an integer of 0, 1, 2, or 3; R^{a} and R^{b} are each independently hydrogen, halo, cyano, hydroxyl, or alkoxy; and X is —O—, —S—, —C(O)—, —C(O)O—, —OC(O)O—, —C(O)NR¹⁴—, —C(═NR¹⁴)NR¹⁵—, —NR¹⁴C(O)NR¹⁵—, —NR¹⁴C(═NR¹⁵)NR¹⁶—, —NR¹⁴S(O)ₖR¹⁵—, —NR¹⁴S(O)ₖNR¹⁵—, —S(O)ₖ—, —S(O)ₖNR¹⁴—, —P(O)OR¹⁴—, or —OP(O)OR¹⁴—; where R¹⁴, R¹⁵, and R¹⁶ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and each k is independently an integer of 1 or 2;
Q¹ is —O—, —N(R¹⁷)—, —C(R¹⁸R¹⁹)—, or —CR¹⁷(NR¹⁸R¹⁹)—; wherein:
R¹⁷ and R¹⁸ are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; and
R¹⁹ is hydrogen, C₁₋₆ alkyl, C₂₋₆ alkenyl, C₂₋₆ alkynyl, C₃₋₇ cycloalkyl, C₆₋₁₄ aryl, heterocyclyl, heteroaryl, —C(O)R²⁰, —C(O)OR²⁰, —C(O)NR²¹R²², —C(═NR²⁰)NR²¹R²², or —S(O)ₘR²⁰; where R²⁰, R²¹, and R²² are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R²¹ and R²² are linked together with the N atom to which they are attached to form heterocyclyl or heteroaryl; and m is an integer of 0, 1, or 2; or
R¹⁸ and R¹⁹ are linked together with the C or N atom to which they are attached to form cycloalkyl, heterocyclyl, or heteroaryl; and
Q² is C₃₋₉ alkylene, C₃₋₉ alkenylene, or C₃₋₉ alkynylene, each optionally containing one to three heteroatoms in the chain of the alkylene, independently selected from O, N, and S;
wherein each alkyl, alkylene, alkenyl, alkenylene, alkynyl, alkynylene, cycloalkyl, cycloalkylene, aryl, heteroaryl, and heterocyclyl is independently, optionally substituted with one or more substituents Q, each Q independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} are linked together to form heterocyclyl, along with the N atom to which they are attached.

2. The compound of claim 1, having the structure of Formula II: 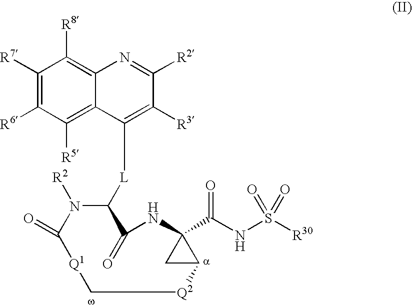
wherein:
R^{2′}, R^{3′}, R^{5′}, R^{6′}, R^{7′}, and R^{8′} are each independently:
hydrogen, halo, cyano, trifluoromethyl, or nitro;
C₁₋₆ alkyl, C₂₋₆ alkenyl, C₂₋₆ alkynyl, C₃₋₇ cycloalkyl, C₆₋₁₄ aryl, heteroaryl, or heterocyclyl; or
—C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═O—NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, —S(O)₂R^{a}, or —S(O)₂NR^{b}R^{c}; wherein R^{a}, R^{b}, R^{c}, and R^{d} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{b} and R^{c} are linked together to form heterocyclyl or heteroaryl, along with the N atom to which they are attached;
wherein each alkyl, alkenyl, alkynyl, cycloalkyl, aryl, heteroaryl, and heterocyclyl is independently, optionally substituted with one or more substituents Q, each Q independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, and R^{h} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} are linked together to form heterocyclyl, along with the N atom to which they are attached.

3. The compound of claim 1, wherein Q² is C₃₋₉ alkylene.

4. The compound of claim 1, wherein Q² is C₃₋₉ alkenylene or alkynylene.

5. The compound of claim 1, wherein Q² is selected from the group consisting of: 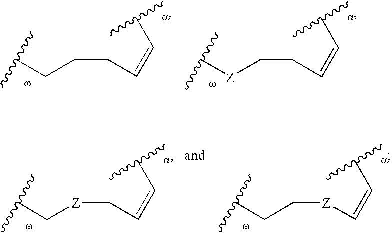
wherein:
Z is —O—, —S—, or —N(R^{Z})—, where R^{Z} is hydrogen, C₁₋₆ alkyl, aryl, heteroaryl, heterocyclyl, —C(O)R^{Za}, —C(O)OR^{Za}, —C(O)NR^{Zb}R^{Zc}, —S(O)₂NR^{Zb}R^{Zc}, or —S(O)₂R^{Za}; and
R^{Za}, R^{Zb}, and R^{Zc} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or
R^{Zb} and R^{Zc} together with the N atom to which they are attached form heterocyclyl or heteroaryl;
wherein each alkyl, alkenyl, alkynyl, cycloalkyl, aryl, heteroaryl, and heterocyclyl is independently, optionally substituted with one or more substituents Q, each Q independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{e}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} are linked together to form heterocyclyl, along with the N atom to which they are attached.

6. The compound of claim 1 having Formula III: 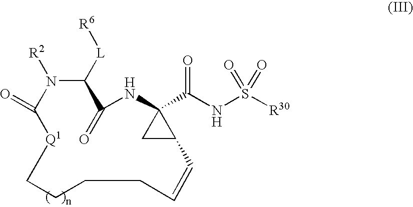
wherein n is an integer ranging from 0 to 5.

7. The compound of claim 2 having the structure of Formula IV: 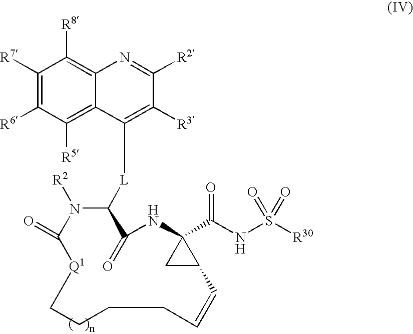
wherein n is an integer ranging from 0 to 5.

8. The compound of claim 7 having the structure of Formula V: 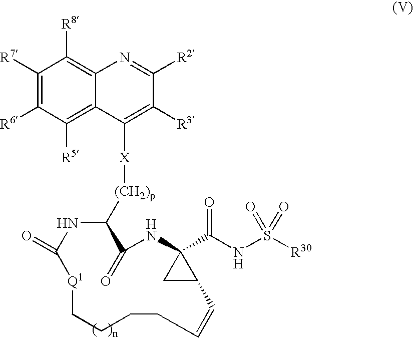
wherein p is an integer ranging from 1 to 5.

9. The compound of claim 8, wherein X is —O—.

10. The compound of claim 8, wherein X is —C(O)O—, wherein the carbon of X is attached to the quinoline group.

11. The compound of claim 8, wherein X is —C(O)NR¹⁴—, wherein the carbon of X is attached to the quinoline group.

12. The compound of claim 11, wherein R¹⁴ is hydrogen, C₁₋₆ alkyl, or C₃₋₇ cycloalkyl, where alkyl and cycloalkyl are each independently, optionally substituted with one or more substituents Q.

13. The compound of claim 11, wherein R¹⁴ is hydrogen.

14. The compound of claim 6, wherein n is 0, 1, or 2.

15. The compound of claim 14, wherein n is 1.

16. The compound of claim 1, wherein R⁶ is C₁₋₆ alkyl, C₃₋₇ cycloalkyl, C₆₋₁₄ aryl, heteroaryl, or heterocyclyl, each optionally substituted with one or more substituents Q.

17. The compound of claim 16, wherein R⁶ is C₆₋₁₄ aryl, heteroaryl, or heterocyclyl, each optionally substituted with one or more substituents Q.

18. The compound of claim 16, wherein R⁶ is selected from the group consisting of: 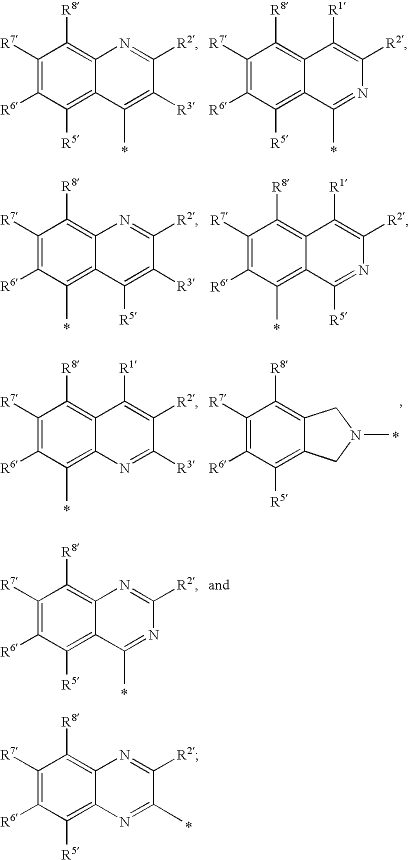
wherein:
R^{2′}, R^{3′}, R^{5′}, R^{6′}, R^{7′}, and R^{8′} are each independently:
hydrogen, halo, cyano, trifluoromethyl, or nitro;
C₁₋₆ alkyl, C₂₋₆ alkenyl, C₂₋₆ alkynyl, C₃₋₇ cycloalkyl, C₆₋₁₄ aryl, heteroaryl, or heterocyclyl; or
—C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═O—NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, —S(O)₂R^{a}, or —S(O)₂NR^{b}R^{c}; wherein R^{a}, R^{b}, R^{c}, and R^{d} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{b} and R^{c} are linked together to form heterocyclyl or heteroaryl, along with the N atom to which they are attached;
wherein each alkyl, alkenyl, alkynyl, cycloalkyl, aryl, heteroaryl, and heterocyclyl is independently, optionally substituted with one or more substituents Q.

19. The compound of claim 2, wherein R^{2′} is C₁₋₆ alkyl, C₂₋₆ alkenyl, C₂₋₆ alkynyl, C₃₋₇ cycloalkyl, C₆₋₁₄ aryl, heterocyclyl, or heteroaryl, each optionally substituted.

20. The compound of claim 19, wherein R^{2′} is C₆₋₁₄ aryl, heterocyclyl, or heteroaryl, each optionally substituted.

21. The compound of claim 2, wherein R^{2′} is selected from the group consisting of: 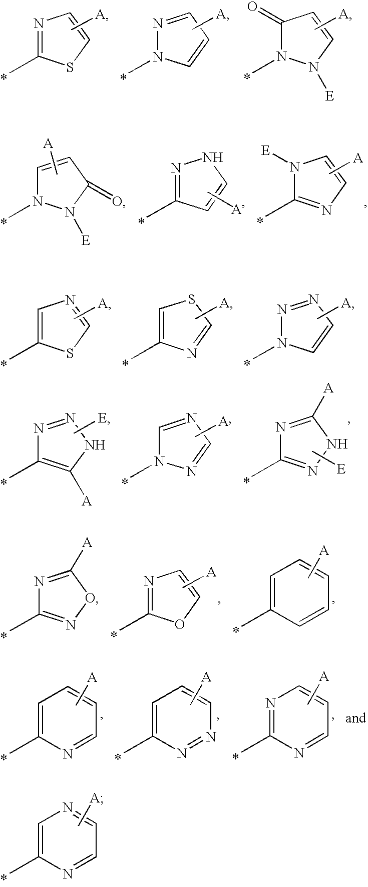
A is hydrogen, halo, cyano, nitro, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, NR^{a}S(O)₂NR^{b}R^{c}, —SR^{a}, —S(O)R^{a}, —S(O)₂R^{a}, or —S(O)₂NR^{b}R^{c};
E is hydrogen, C₁₋₆ alkyl, C₂₋₆ alkenyl, C₂₋₆ alkynyl, C₃₋₇ cycloalkyl, C₆₋₁₄ aryl, heteroaryl, heterocyclyl, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═O—NR^{a})NR^{b}R^{c}, OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, —S(O)₂R^{a}, or —S(O)₂NR^{b}R^{c}; and
R^{a}, R^{b}, R^{c}, and R^{d} are each independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{b} and R^{c} together with the N atom to which they are attached form heterocyclyl or heteroaryl;
wherein each alkyl, alkenyl, alkynyl, cycloalkyl, aryl, heteroaryl, and heterocyclyl is optionally substituted with one or more substituents Q.

22. The compound of claim 21, wherein A is hydrogen, halo, cyano, nitro, C₁₋₆ alkyl, C₂₋₆ alkenyl, C₂₋₆ alkynyl, C₆₋₁₄ aryl, C₃₋₇ cycloalkyl, heteroaryl, or heterocyclyl, wherein C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

23. The compound of claim 21, wherein A is hydrogen, fluoro, methyl, ethyl, n-propyl, isopropyl, cyclopropyl, isobutyl, isopentyl, trifluoromethyl, benzyl, 2-morpholin-4-yl-ethyl, cyclobutyl, ethynyl, methoxy, ethoxy, or isopropylamino.

24. The compound of claim 21, wherein A is hydrogen, methyl, isopropyl, isobutyl, trifluoromethyl, cyclopropyl, cyclobutyl, ethynyl, methoxy, ethoxy, or isopropylamino.

25. The compound of claim 21, wherein E is hydrogen, cyano, C₁₋₆ alkyl, C₂₋₆ alkenyl, C₂₋₆ alkynyl, C₃₋₇ cycloalkyl, C₆₋₁₄ aryl, heterocyclyl, or heteroaryl, wherein C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

26. The compound of claim 21, wherein E is hydrogen, methyl, ethyl, n-propyl, isopropyl, cyclopropyl, isobutyl, isopentyl, trifluoromethyl, benzyl, 2-morpholin-4-yl-ethyl, cyclobutyl, ethynyl, methoxy, ethoxy, or isopropylamino.

27. The compound of claim 21, wherein E is hydrogen, methyl, ethyl, n-propyl, isopropyl, isobutyl, isopentyl, benzyl, or 2-morpholin-4-yl-ethyl.

28. The compound of claim 20, wherein R^{2′} is selected from the group consisting of:

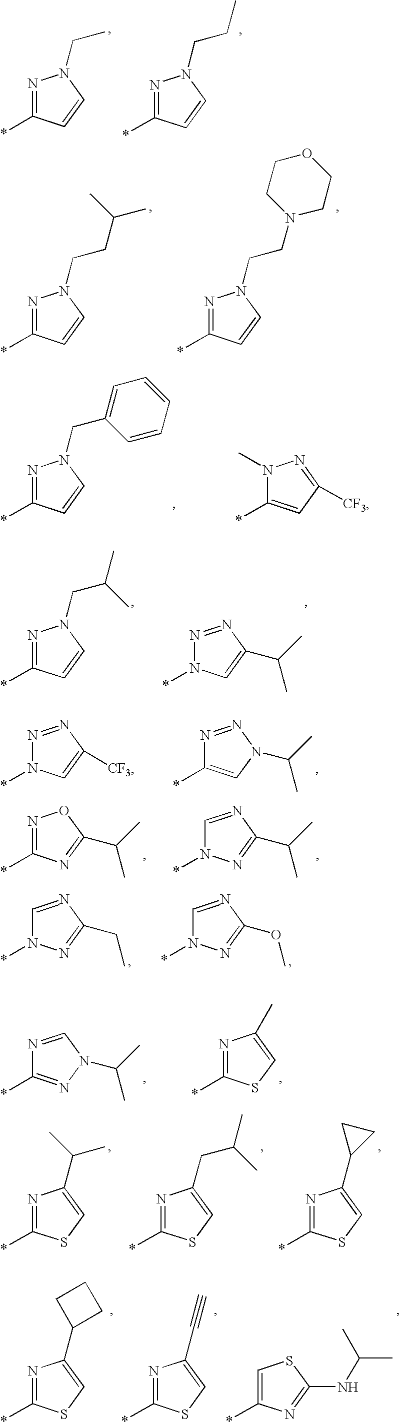

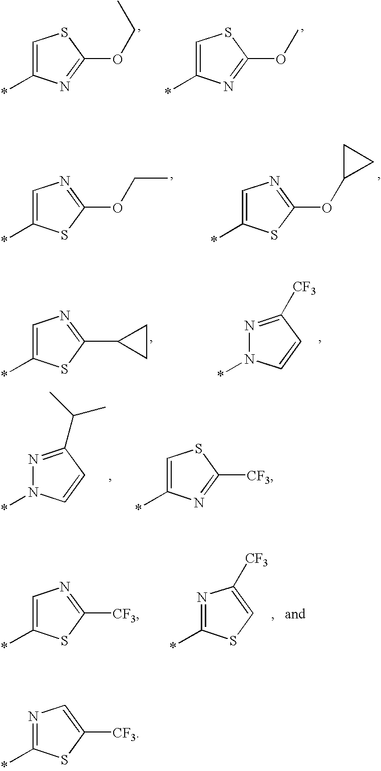

29. The compound of claim 2, wherein R^{3′} is hydrogen, hydroxy, cyano, halo, C₁₋₆ alkyl, C₂₋₆ alkenyl, C₂₋₆ alkynyl, C₃₋₇ cycloalkyl, C₆₋₁₄ aryl, C₁₋₆ alkoxy, or C₃₋₇ cycloalkoxy, wherein C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

30. The compound of claim 29, wherein R^{3′} is hydrogen.

31. The compound of claim 2, wherein R^{5′} is hydrogen, hydroxy, cyano, halo, C₁₋₆ alkyl, C₂₋₆ alkenyl, C₂₋₆ alkynyl, C₃₋₇ cycloalkyl, C₆₋₁₄ aryl, C₁₋₆ alkoxy, or C₃₋₇ cycloalkoxy, wherein C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

32. The compound of claim 2, wherein R^{5′} is hydrogen or methoxy.

33. The compound of claim 2, wherein R^{6′} is hydrogen, hydroxy, cyano, halo, C₁₋₆ alkyl, C₂₋₆ alkenyl, C₂₋₆ alkynyl, C₃₋₇ cycloalkyl, C₆₋₁₄ aryl, C₁₋₆ alkoxy, or C₃₋₇ cycloalkoxy, wherein C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

34. The compound of claim 2, wherein R^{7′} is hydrogen, cyano, halo, C₁₋₆ alkyl, C₂₋₆ alkenyl, C₂₋₆ alkynyl, C₃₋₇ cycloalkyl, C₆₋₁₄ aryl, heteroaryl, heterocyclyl, —OR^{a}—, —NR^{a}S(O)₂R^{b}, or —S(O)NR^{b}R^{c}, wherein each R^{a}, R^{b}, and R^{c} is independently hydrogen, C₁₋₆ alkyl, C₂₋₆ alkenyl, C₂₋₆ alkynyl, C₃₋₇ cycloalkyl, C₆₋₁₄ aryl, heteroaryl, or heterocyclyl; wherein each C₁₋₆ alkyl, C₂₋₆ alkenyl, C₂₋₆ alkynyl, C₆₋₁₄ aryl, C₃₋₇ cycloalkyl, heteroaryl, and heterocyclyl are each optionally substituted with one or more substituents Q.

35. The compound of claim 34, wherein R^{7′} is —OR^{a}.

36. The compound of claim 34, wherein R^{7′} is difluoromethyl or methoxy.

37. The compound of claim 2, wherein R^{8′} is hydrogen, halo, cyano, C₁₋₆ alkyl, or C₃₋₇ cycloalkyl; wherein C₁₋₆ alkyl and C₃₋₇ cycloalkyl are each optionally substituted with one or more substituents Q.

38. The compound of claim 37, wherein R^{8′} is hydrogen, fluoro, chloro, bromo, or methyl.

39. The compound of claim 1, wherein L is C₁₋₆ alkylene, optionally substituted with one or more substituents Q.

40. The compound of claim 39, wherein L is —(CH₂)ₚ—.

41. The compound of claim 40, wherein L is —CH₂—.

42. The compound of claim 40, wherein L is —CH₂CH₂—.

43. The compound of claim 1, wherein L is —(CR^{a}R^{b})ₚX—, wherein X is —O—, —C(O)—, —OC(O)—, —C(O)NR¹⁴—, —OC(O)NR¹⁴—, —S(O)ₖ—, —S(O)ₖNR¹⁴—, or —NR¹⁴S(O)ₖ—.

44. The compound of claim 43, wherein each R^{a} and R^{b} is independently hydrogen or fluoro.

45. The compound of claim 1, wherein Q¹ is —O—.

46. The compound of claim 1, wherein Q¹ is —N(R¹⁷)—.

47. The compound of claim 46, wherein R¹⁷ is hydrogen, C₁₋₆ alkyl, C₃₋₇ cycloalkyl, heterocyclyl, or heteroaryl, wherein C₁₋₆ alkyl, C₃₋₇ cycloalkyl, heterocyclyl, and heteroaryl are each optionally substituted with one or more substituents Q.

48. The compound of claim 46, wherein R¹⁷ is hydrogen, C₁₋₆ alkyl, or C₃₋₇ cycloalkyl, wherein C₁₋₆ alkyl and C₃₋₇ cycloalkyl are each optionally substituted with one or more substituents Q.

49. The compound of claim 48, wherein R¹⁷ is hydrogen or methyl.

50. The compound of claim 1, wherein Q¹ is —C(R¹⁸R¹⁹)—.

51. The compound of claim 50, wherein R¹⁸ and R¹⁹ are each independently hydrogen, C₁₋₆ alkyl, or C₃₋₇ cycloalkyl, wherein C₁₋₆ alkyl and C₃₋₇ cycloalkyl are each optionally substituted with one or more substituents Q.

52. The compound of claim 51, wherein R¹⁸ and R¹⁹ are hydrogen.

53. The compound of claim 1, wherein Q¹ is —CR¹⁷(NR¹⁸R¹⁹)—.

54. The compound of claim 53, wherein R¹⁷ and R¹⁸ are each independently hydrogen; C₁₋₆ alkyl, or C₃₋₇ cycloalkyl, wherein C₁₋₆ alkyl and C₃₋₇ cycloalkyl are each optionally substituted with one or more substituents Q.

55. The compound of claim 54, wherein R¹⁷ is hydrogen.

56. The compound of claim 54, wherein R¹⁸ is hydrogen or methyl.

57. The compound of claim 54, wherein R¹⁹ is hydrogen, —C(O)R²⁰, —C(O)OR²⁰, —C(O)NR²¹R²², —C(═NR²⁰)NR²¹R²², or —SO₂R²⁰.

58. The compound of claim 57, wherein R¹⁹ is —C(O)OR²⁰.

59. The compound of claim 57, wherein R²⁰ is C₁₋₆ alkyl, C₃₋₇ cycloalkyl, C₆₋₁₄ aryl, heterocyclyl, or heteroaryl, each optionally substituted with one or more substituents Q.

60. The compound of claim 59, wherein R²⁰ is t-butyl or benzyl.

61. The compound of claim 1, wherein R³⁰ is C₁₋₆ alkyl, C₂₋₆ alkynyl, C₆₋₁₄ aryl, C₃₋₇ cycloalkyl, C₁₋₆ alkyl-C₃₋₇ cycloalkylene, or heterocyclyl, each optionally substituted with one or more substituents Q.

62. The compound of claim 61, wherein R³⁰ is C₂₋₆ alkynyl or C₃₋₇ cycloalkyl, each optionally substituted with one or more substituents Q.

63. The compound of claim 62, wherein R³⁰ is propargyl, cyclopropyl, 1-methylcyclopropyl, cyclobutyl, cyclopentyl, or cyclohexyl, each optionally substituted with one or more substituents Q.

64. The compound of claim 62, wherein R³⁰ is propargyl, cyclopropyl, 1-methylcyclopropyl, cyclobutyl, cyclopentyl, or cyclohexyl.

65. The compound of claim 1, wherein R² is hydrogen or C₁₋₆ alkyl, optionally substituted.

66. The compound of claim 65, wherein R² is hydrogen.

67. The compound of claim 1 having the structure of Formula VII:

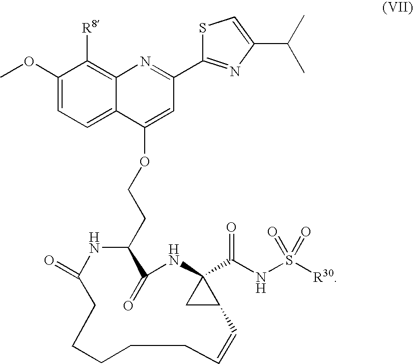

68. The compound of claim 1 having the structure of Formula VIII:

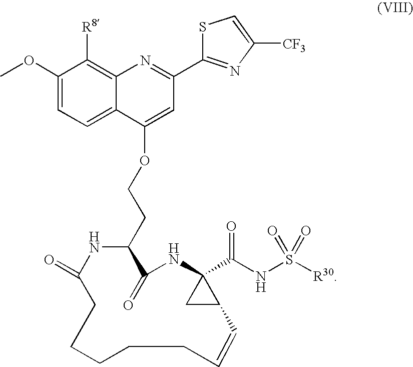

69. The compound of claim 1 having the structure of Formula IX:

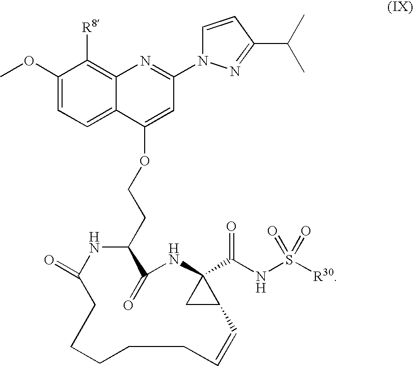

70. The compound of claim 1 having the structure of Formula X: 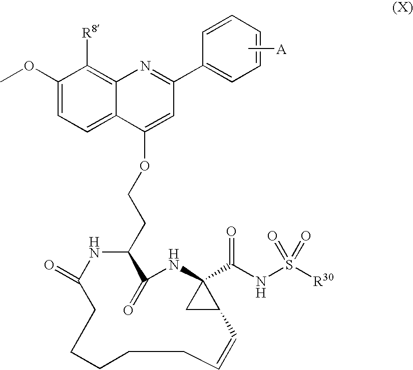
wherein A is hydrogen or fluoro.

71. The compound of claim 1 having the structure of Formula XI:

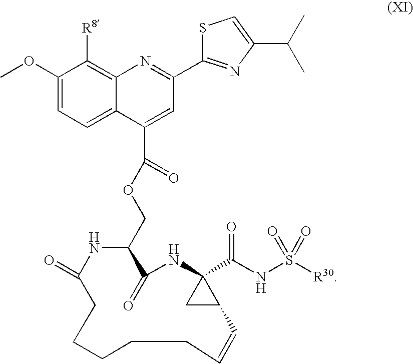

72. The compound of claim 1 having the structure of Formula XII:

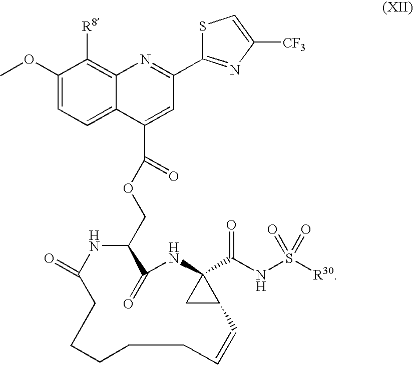

73. The compound of claim 1 having the structure of Formula XIII:

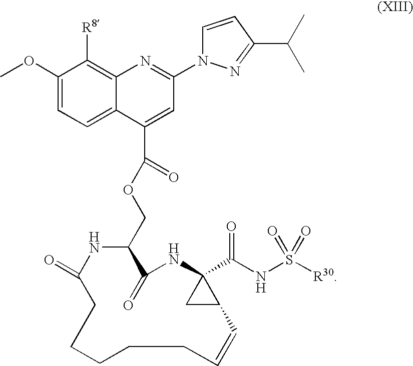

74. The compound of claim 1 having the structure of Formula XIV: 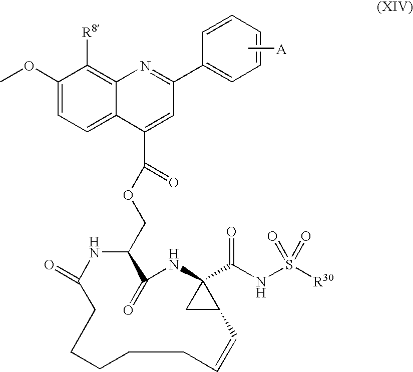
wherein A is hydrogen or fluoro.

75. The compound of claim 67, wherein R^{8′} and R³⁰ are selected from Table 1:
TABLE 1

| R^{8′} | R³⁰ |
|---|---|
| H | Cyclopropyl |
| H | 1-Methylcyclopropyl |
| H | Cyclobutyl |
| H | Cyclopentyl |
| H | Cyclohexyl |
| H | Aminomethyl |
| Methyl | Cyclopropyl |
| Methyl | 1-Methylcyclopropyl |
| Methyl | Cyclobutyl |
| Methyl | Cyclopentyl |
| Methyl | Cyclohexyl |
| Methyl | Aminomethyl |
| Cl | Cyclopropyl |
| Cl | 1-Methylcyclopropyl |
| Cl | Cyclobutyl |
| Cl | Cyclopentyl |
| Cl | Cyclohexyl |
| Cl | Aminomethyl |
| F | Cyclopropyl |
| F | 1-Methylcyclopropyl |
| F | Cyclobutyl |
| F | Cyclopentyl |
| F | Cyclohexyl |
| F | Aminomethyl |
| Br | Cyclopropyl |
| Br | 1-Methylcyclopropyl |
| Br | Cyclobutyl |
| Br | Cyclopentyl |
| Br | Cyclohexyl |
| Br | Aminomethyl |

76. The compound of claim 1 selected from the group consisting of: 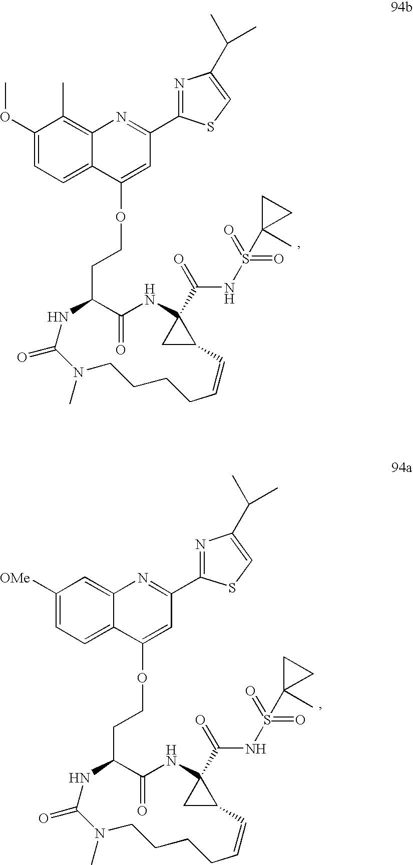 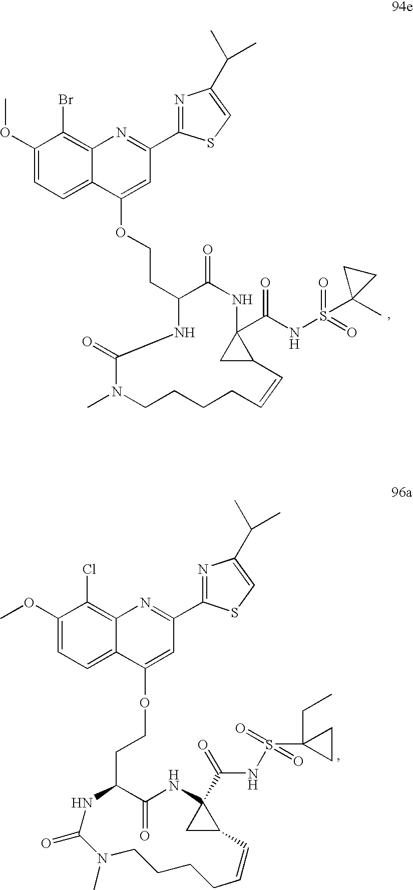 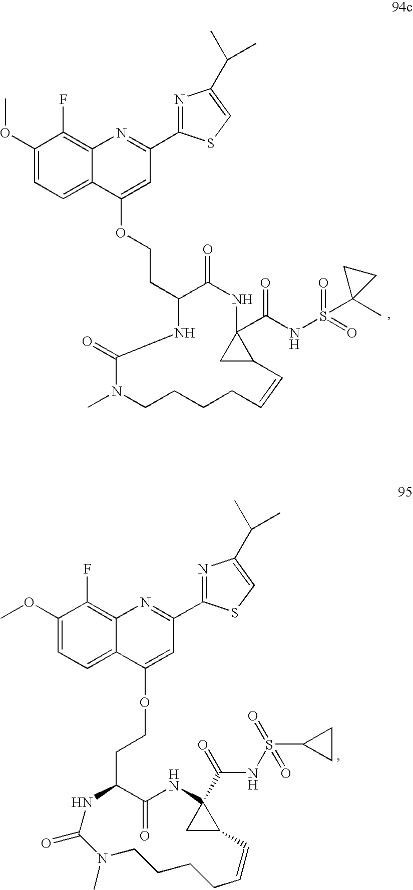 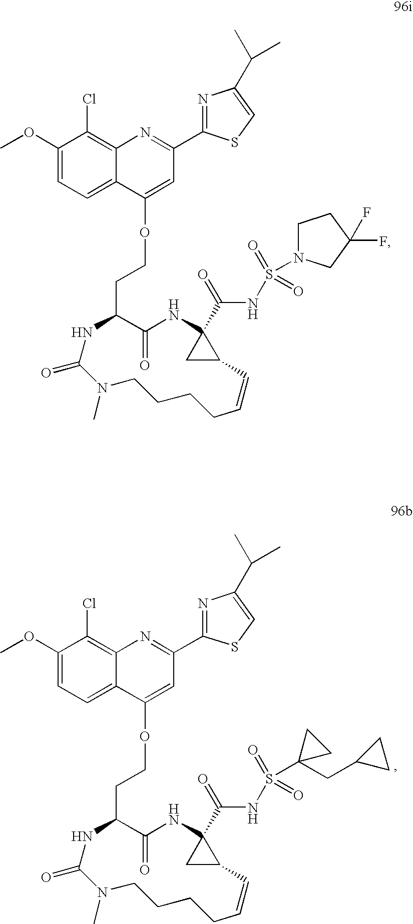 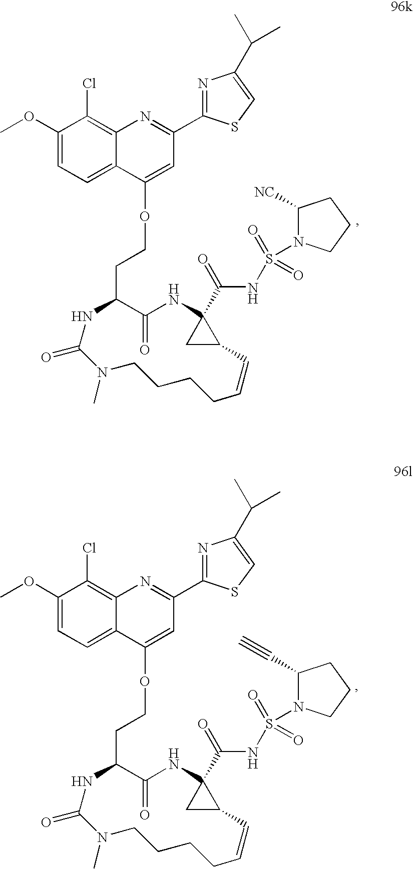 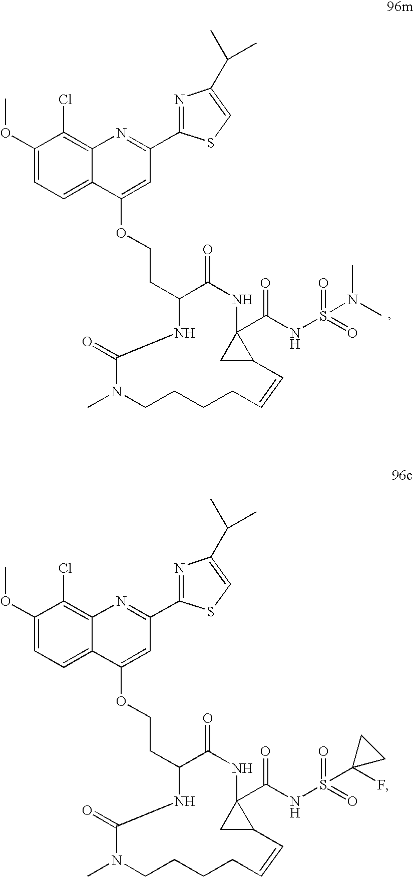 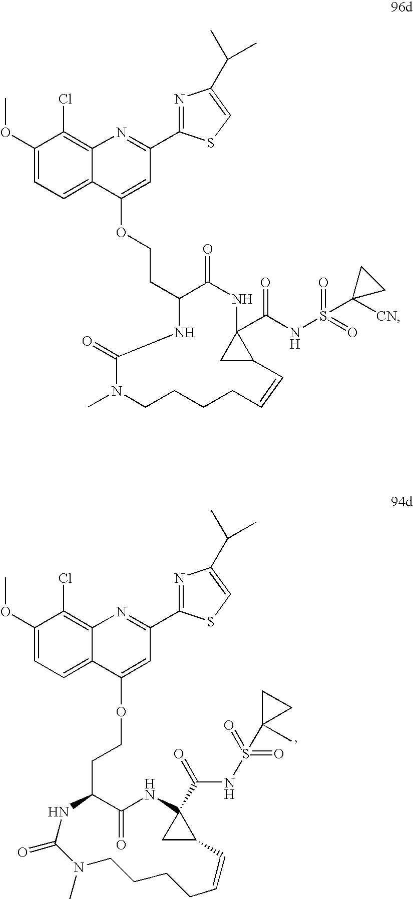 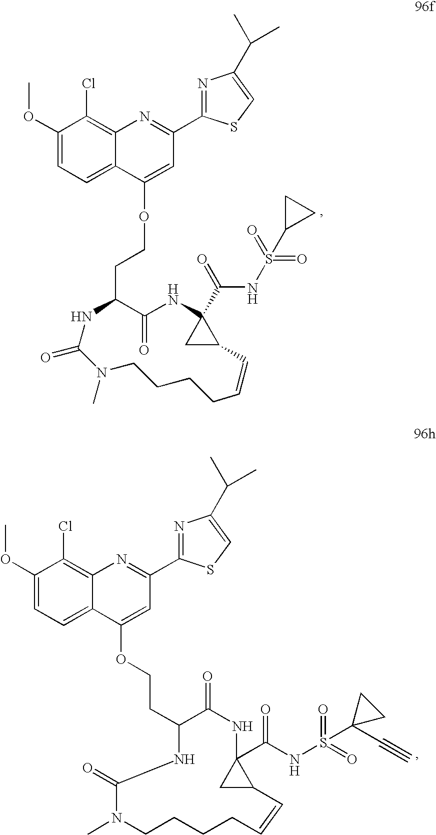 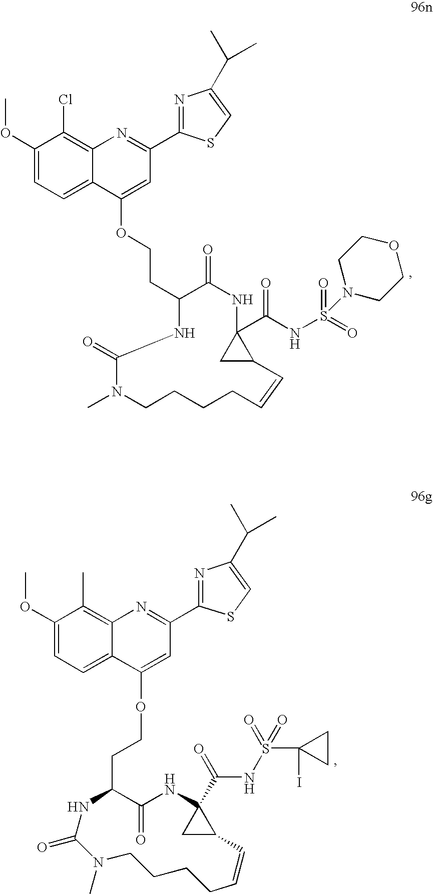 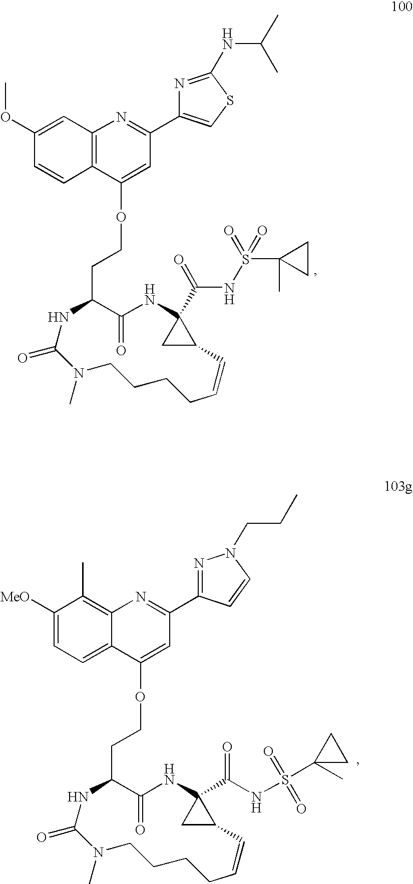 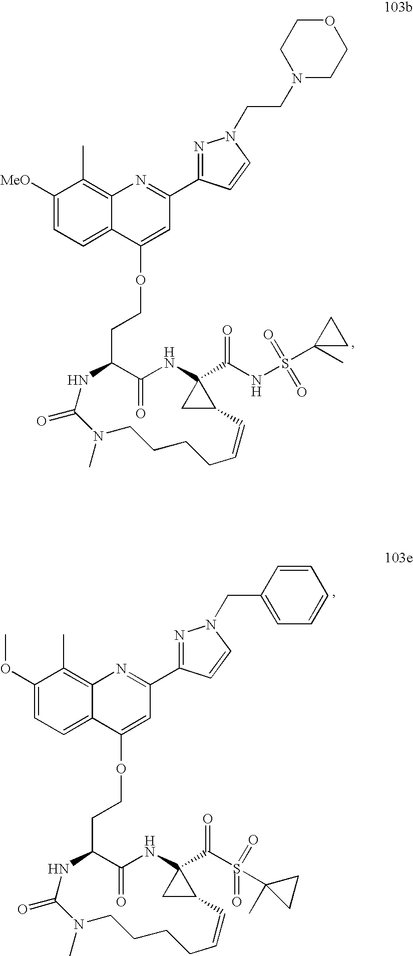 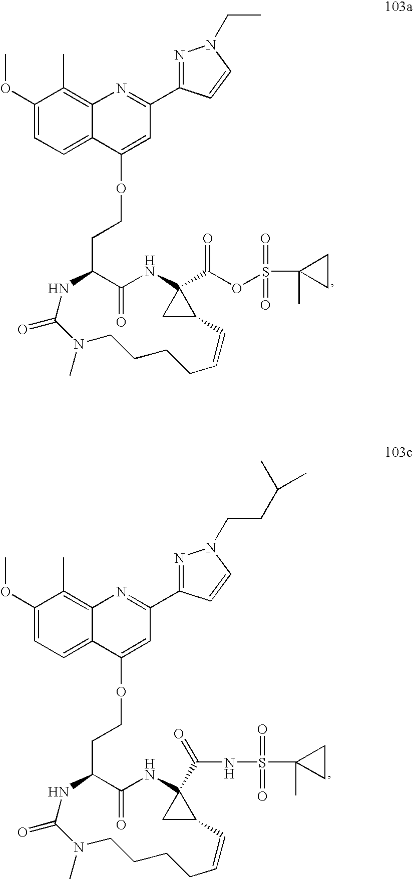 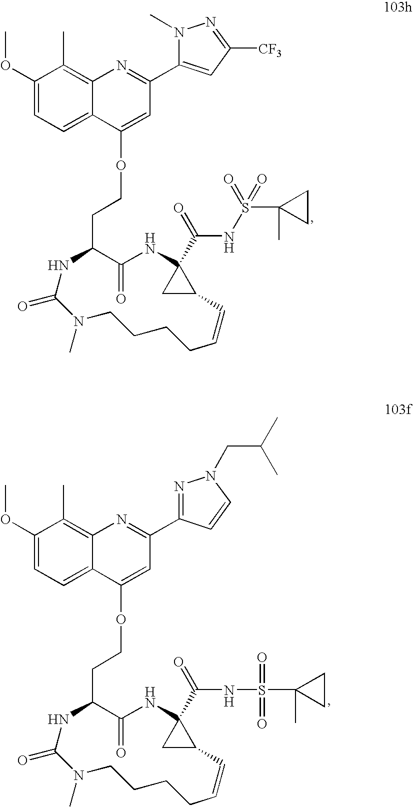 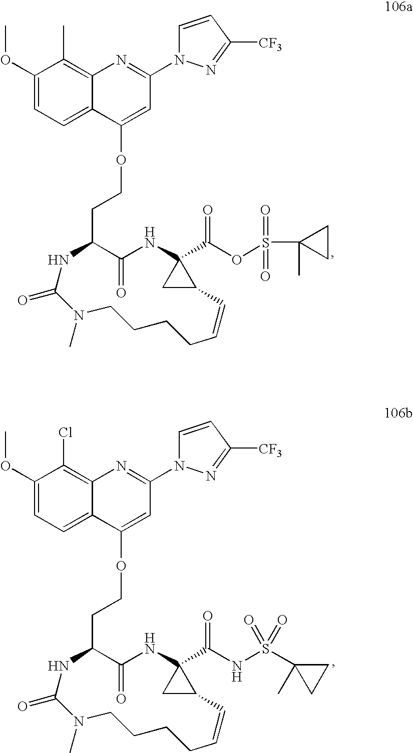 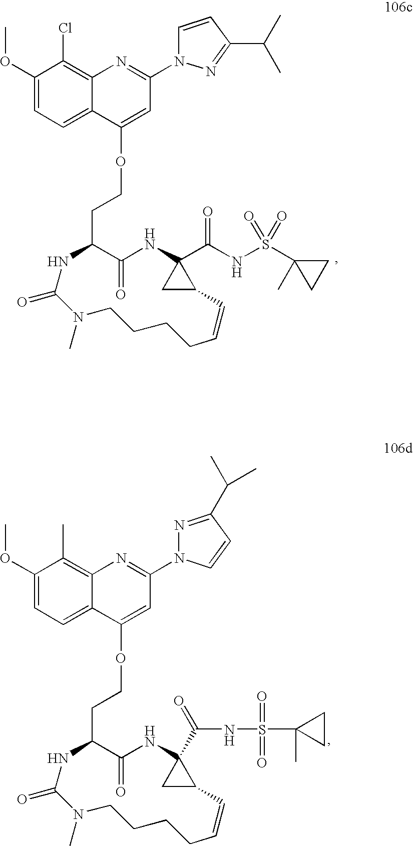 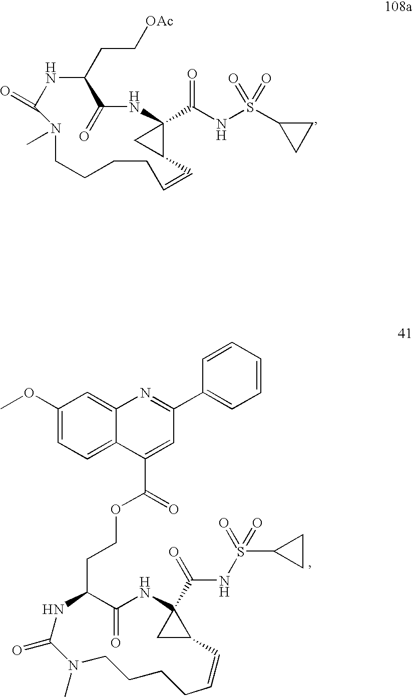 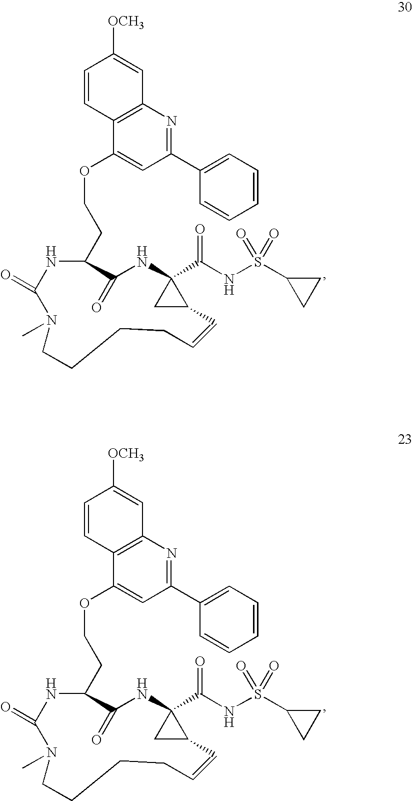 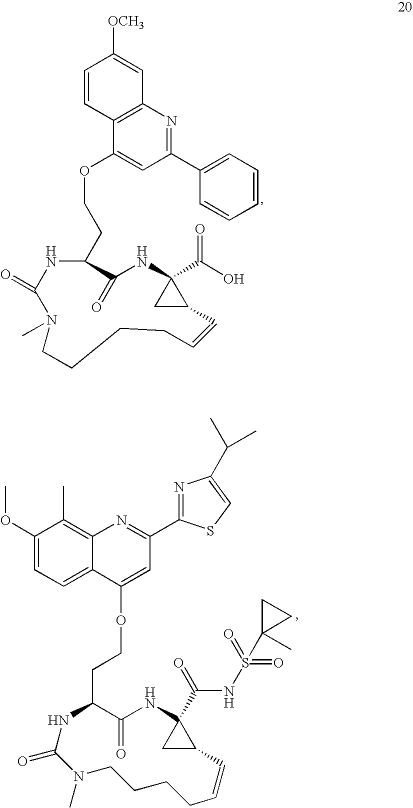 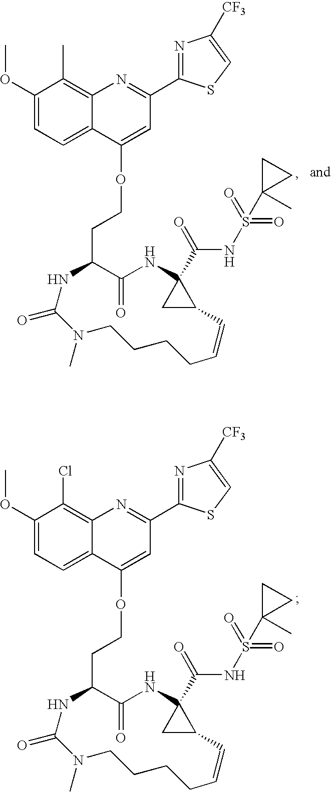
and pharmaceutically acceptable salts, solvates, and prodrugs thereof.

77. A pharmaceutical composition comprising a compound of claim 1, and one or more pharmaceutically acceptable excipients or carriers.

78. The pharmaceutical composition of claim 77, further comprising a second antiviral agent.

79. The pharmaceutical composition of claim 78, wherein the second antiviral agent is selected from the group consisting of an interferon, ribavirin, an interleukin, an NS3 protease inhibitor, a cysteine protease inhibitor, a phenathrenequinone, a thiazolidine, a benzanilide, a helicase inhibitor, a polymerase inhibitor, a nucleotide analogue, a liotoxin, acerulenin, an antisense phosphorothioate ologodeoxynucleotide, an inhibitor of IRES-dependent translation, and a ribozyme.

80. The pharmaceutical composition of claim 78, wherein the second antiviral agent is an interferon.

81. The pharmaceutical composition of claim 80, wherein the interferon is selected from the group consisting of pegylated interferon alpha 2a, interferon alphcon-1, natural interferon, albuferon, interferon beta-1a, omega interferon, interferon alpha, interferon gamma, interferon tau, interferon delta, and interferon gamma-1b.

82. The pharmaceutical composition of claim 77, wherein the composition is formulated for single dose administration.

83. The pharmaceutical composition of claim 77, wherein the composition is formulated as oral, parenteral, or intravenous dosage form.

84. The pharmaceutical composition of claim 83, wherein the oral dosage form is a tablet or capsule.

85. The pharmaceutical composition of claim 77, wherein the compound is administered in a dose of about 0.5 milligram to about 1,000 milligram daily.

86. A method for treating or preventing an HCV infection, which comprises administering the compound of claim 1.

87. A method of treating, preventing, or ameliorating one or more symptoms of a liver disease or disorder associated with an HCV infection, comprising administering a compound of claim 1.

88. The method of claim 86, wherein the method comprises administering a second antiviral agent, in combination or alternation.

89. The method of claim 88, wherein the second antiviral agent is selected from the group consisting of an interferon, ribavirin, amantadine, an interleukin, a NS3 protease inhibitor, a cysteine protease inhibitor, a phenathrenequinone, a thiazolidine, a benzanilide, a helicase inhibitor, a polymerase inhibitor, a nucleotide analogue, a liotoxin, acerulenin, an antisense phosphorothioate ologodeoxynucleotide, an inhibitor of IRES-dependent translation, and a ribozyme.

90. The method of claim 88, wherein the second antiviral agent is an interferon.

91. The method of claim 90, wherein the interferon is selected from the group consisting of pegylated interferon alpha 2a, interferon alphcon-1, natural interferon, albuferon, interferon beta-1a, omega interferon, interferon alpha, interferon gamma, interferon tau, interferon delta, and interferon gamma-1b.

92. A method for inhibiting replication of a virus in a host, which comprises contacting the host with a compound of claim 1.

93. The method of claim 92, wherein the host is a human.

94. A method for inhibiting replication of a virus, which comprises contacting the virus with a compound of claim 1.

95. A method for inhibiting the activity of a serine protease, which comprises contacting the protease with a compound of claim 1.

96. The method of claim 95, wherein the serine protease is an HCV NS3 protease.
